(19) United States
(12) Patent Application Publication
(10) Pub. No.: US 2026/0088126 A1
(43) Pub. Date: Mar. 26, 2026

(54) METHOD AND DEVICE FOR PREDICTING PRIME EDITING EFFICIENCY OF VARIOUS PRIME EDITORS IN DIFFERENT CELL TYPES

(71) Applicant: UIF (UNIVERSITY INDUSTRY FOUNDATION), YONSEI UNIVERSITY, Seoul (KR)

(72) Inventors: Hyongbum KIM, Seoul (KR); Goosang YU, Incheon (KR); Hui Kwon KIM, Suwon-si (KR); Jinman PARK, Seoul (KR)

(73) Assignee: UIF (UNIVERSITY INDUSTRY FOUNDATION), YONSEI UNIVERSITY, Seoul (KR)

(21) Appl. No.: 19/109,301

(22) PCT Filed: Aug. 29, 2023
(86) PCT No.: PCT/KR2023/012794

§ 371 Date: Mar. 6, 2025

(30) Foreign Application Priority Data
Sep. 7, 2022 (KR) 10-2022-0113791
Aug. 24, 2023 (KR) 10-2023-0111504

(51) Int. Cl. G16B 30/00; C12N 9/12; C12N 9/22; C12N 15/10; C12N 15/11; C12Q 1/6869; G16B 25/00; G16B 40/20

(52) U.S. Cl. CPC G16B 30/00; C12N 9/1276; C12N 9/226; C12N 15/1082; C12N 15/111; C12Q 1/6869; G16B 25/00; G16B 40/20; C07K 2319/80; C12N 2310/20; C12Y 207/07049

(57) ABSTRACT

Provided are a method for training a predictive model for prime editing efficiency of pegRNA for various cell types and various prime editor types, and a method and apparatus for predicting prime editing efficiency using the prime-editing efficiency predictive model trained by the same method. In addition, a method for training a predictive model for off-target prime editing efficiency, and a method and apparatus for predicting off-target prime editing efficiency using the predictive model for off-target prime editing efficiency trained by the method.

### TECHNICAL FIELD

The present disclosure relates to a method for training a predictive model for prime editing efficiency for various cell types and various types of prime editors, to a method for predicting a prime editing efficiency using the predictive model for prime editing efficiency trained by the method, and to an apparatus for predicting prime editing efficiency. In addition, the present disclosure relates to a method for training a predictive model for off-target prime editing efficiency, to a method for predicting an off-target prime editing efficiency using the predictive model for off-target prime editing efficiency trained by the method, and to an apparatus for predicting off-target prime editing efficiency.

### BACKGROUND ART

Prime editing allows all 12 possible point mutations, small insertions and deletions (indels), and combinations of these modifications to be introduced into genomic DNA (Anzalone, A. V. et al., 2019).

A prime editor (PE) consists of a fusion protein of Cas9 nickase-reverse transcriptase (RT) and a prime editing guide RNA (pegRNA). The pegRNA includes a guide sequence, a tracrRNA scaffold, a reverse transcription template (RTT), and a primer binding site (PBS). Several prime editors have been reported, including PE1, PE2, PE3, PE4, and PE5 (Anzalone et al., 2019; Chen et al., 2021). PE2, a widely used prime editor, is a more efficient version of PE1. PE3 consists of PE2 and an additional single guide RNA (sgRNA). PE3 is often more efficient in intended edits and induces higher levels of unintended indels than PE2. PE4 and PE5 are made as increased efficiency and precision versions of the PE2 and PE3 by combining MLH1dn (a dominant negative form of MLH1), which suppresses the mismatch repair (MMR) system, with PE2 and PE3, respectively. PE2max, PE3max, PE4max, and PE5max are improved versions of PE2, PE3, PE4, and PE5, respectively (Chen et al., 2021).

Designing pegRNAs for efficient prime editing is difficult. Hundreds or thousands of pegRNAs can be designed for a single intended edit. Selecting the most efficient version of the pegRNAs often requires extensive experimentation.
(Non-patent document 1) Anzalone, A. V. et al. (2019). Search-and-replace genome editing without double-strand breaks or donor DNA. Nature 576, 149-157.
(Non-patent document 2) Chen et al. (2021). Enhanced prime editing systems by manipulating cellular determinants of editing outcomes. Cell 184, 5635-5652.e5629.

### DISCLOSURE

### Technical Problem

A lack of efficiency often limits the application of prime editing in many cases. Determining the most efficient pegRNA and prime editor to generate intended edits under various experimental conditions can require significant time and resources. Herein, the present inventors evaluated a prime editing efficiency for a total of 338, 996 pairs of pegRNAs, including 3,979 epegRNAs and target sequences, in an error-free manner. With these datasets, it was possible to systematically determine factors affecting the prime editing efficiency. The present inventors then developed computation models called DeepPrime and DeepPrime-FT. These models can predict the prime editing efficiency for eight prime editing systems in seven cell types for all possible edit types involving up to three base pairs.

The present inventor(s) also extensively profiled the prime editing efficiency in off-targets and developed computation models for predicting editing efficiency in such targets.

Together with our advanced knowledge of the determinants of the prime editing efficiency, these computation models will greatly facilitate prime editing applications.

One aspect is to provide a method for training a predictive model for prime editing efficiency.

Another aspect is to provide a method for predicting prime editing efficiency.

A further aspect is to provide an apparatus for predicting prime editing efficiency.

A yet further aspect is to provide a computer-readable recording medium on which a program is recorded, the program being configured to cause a computer to execute the method for predicting prime editing efficiency.

A still yet further aspect is to provide a method for training a predictive model for off-target prime editing efficiency.

A still yet further aspect is to provide a method for predicting off-target prime editing efficiency.

A still yet further aspect is to provide an apparatus for predicting off-target prime editing efficiency.

A still yet further aspect is to provide a computer-readable recording medium on which a program is recorded, the program being configured to cause a computer to execute the method for predicting off-target prime editing efficiency.

### Technical Solution

One aspect provides a method for training a predictive model for prime editing efficiency.

Specifically, a method for training a predictive model for prime editing efficiency of pegRNAs for various cell types and various types of prime editors is provided. More specifically, a method for training a predictive model for prime editing efficiency of pegRNAs for all edit types of up to 3-nt length in various cell types and various types of prime editors is provided. Even more specifically, a method for training a predictive model for prime editing efficiency of each of the pegRNAs for all edit types of from 1-nt to 3-nt length in 7 cell types and 8 prime editing types is provided.

The method includes obtaining a dataset on the prime editing efficiency of pegRNAs depending on cell types and prime editor types; and training the predictive model using the dataset by deep learning, to establish relationships between the cell types and prime editing efficiency and relationships between the prime editor types and prime editing efficiency.

The term “prime editing” refers to a genome editing method that may introduce genetic changes by cutting only one strand of DNA without cutting double strands of DNA with 4th generation genetic scissors “prime editor (PE)”.

The term “prime editor” may be used interchangeably with “prime editing system”. The prime editor contains a fusion protein of Cas9 nickase-reverse transcriptase (RT) and a prime editing guide RNA (pegRNA). In this specification, the prime editor may mean containing only the Cas9 nickase-RT fusion protein. The prime editor may also mean containing both the fusion protein of Cas9 nickase-RT and the pegRNA. For example, when the pegRNA is introduced separately into the cell, the introduction of the prime editor thereto may mean that only the Cas9 nickase-RT fusion protein is introduced. In other words, when the pegRNA has already been introduced, the introduction of the prime editor may mean introducing only the Cas9 nickase-RT fusion protein. In one embodiment, the prime editor may refer to the fusion protein of Cas9 nickase-RT. Herein, the Cas9 nickase may be Cas9 H850A. The “Cas9 nickase” used in the prime editor may be modified to nick a single strand of DNA.

The cell types may include two or more various cell types and are not limited to a specific type and specific number. In another embodiment, the cell types may include two or more selected from the group consisting of HEK293T, HCT116, DLD1, MDA-MB-231, A549, HeLa, and NIH3T3. In a further embodiment, the cell types may include all the HEK293T, HCT116, DLD1, MDA-MB-231, A549, HeLa, and NIH3T3. In a yet further embodiment, the cell types may further include known cells in addition to the HEK293T, HCT116, DLD1, MDA-MB-231, A549, HeLa, and NIH3T3.

Conventional predictive models for prime editing efficiency had limitations in that the models were trained using a dataset on prime-editing efficiency in only one cell type (e.g., HEK293T). However, to use prime editing as a gene editing treatment in the future, it will be inevitably necessary to predict prime editing efficiency for various cells (e.g., various cancer cells, and various animal cells). According to the training method according to the one aspect, the prepared may be a predictive model for prime editing efficiency trained using a dataset on prime editing efficiency in various cell types. When using the resulting predictive model, prime editor types and pegRNAs that show high prime editing efficiency in various cell types may be selected without individual experiments.

The prime editor types may include two or more various prime editor types and are not limited to a specific type or specific number. In a still yet further embodiment, the prime editor types may include two or more selected from the group consisting of PE2, PE2max, PE2max-e, PE4max, PE4max-e, NRCH-PE2, NRCH-PE2max, and NRCH-PE4max. In a still yet further embodiment, the prime editor types may include all the PE2, PE2max, PE2max-e, PE4max, PE4max-e, NRCH-PE2, NRCH-PE2max, and NRCH-PE4max. In a still yet further embodiment, the prime editor types may further include a known prime editor or a prime editor that may be newly developed in the future in addition to the PE2, PE2max, PE2max-e, PE4max, PE4max-e, NRCH-PE2, NRCH-PE2max, and NRCH-PE4max.

According to the training method according to another aspect, the prepared may be a predictive model for prime editing efficiency trained using a dataset on prime editing efficiency, which is induced by various types of prime editors in various cell types. In other words, according to the training method according to one aspect, a predictive model for the prime editing efficiency of each of pegRNAs depending on combination of various cell types and various prime editor types may be prepared.

In a still yet further embodiment, the dataset may include two or more datasets on prime editing efficiency, which is induced by a specific prime editor type in a specific cell type. Illustratively, the dataset may include two or more, or all 18, of the following data sets:
1) dataset on prime editing efficiency induced by PE2max in A549 cells;
2) dataset on prime editing efficiency induced by PE2max-e in A549 cells;
3) dataset on prime editing efficiency induced by PE4max in A549 cells;
4) dataset on prime editing efficiency induced by PE4max-3 in A549 cells;
5) dataset on prime editing efficiency induced by NRCH-PE4max in DLD1 cells;
6) dataset on prime editing efficiency induced by PE2max in DLD1 cells;
7) dataset on prime editing efficiency induced by PE4max in DLD1 cells;
8) dataset on prime editing efficiency induced by PE2 in HCT116 cells;
9) dataset on prime editing efficiency induced by NRCH-PE2 in HEK293T cells;
10) dataset on prime editing efficiency induced by NRCH-PE2max in HEK293T cells;
11) dataset on prime editing efficiency induced by PE2 in HEK293T cells;
12) dataset on prime editing efficiency induced by PE2max in HEK293T cells;
13) dataset on prime editing efficiency induced by PE2max-e in HEK293T cells;
14) dataset on prime editing efficiency induced by PE4max in HEK293T cells;
15) dataset on prime editing efficiency induced by PE4max-e in HEK293T cells;
16) dataset on prime editing efficiency induced by PE2max in Hela cells;
17) dataset on prime editing efficiency induced by PE2 in MDA-MB-231 cells; and
18) dataset on prime editing efficiency induced by NRCH-PE4max in NIH3T3 cells.

The term “Prime editing efficiency” refers to gene editing efficiency by a prime editor and a pegRNA. The prime editing efficiency may refer to a ratio of edits induced by a pegRNA within a target sequence without unintended mutations. The prime editing efficiency may be expressed as a percentage.

The term “dataset on prime editing efficiency” may be conventional known data, or may be data directly obtained by any method that may be appropriately adopted by those skilled in the art. As long as the dataset on prime editing efficiency is data that may create a predictive model for prime editing efficiency, there are no limitations in how data is obtained. In a still yet further embodiment, the dataset on prime editing efficiency may be a dataset on prime editing efficiency, which is analyzed using a pegRNA and its corresponding target sequence through a high-throughput experiment.

Specifically, the dataset on the prime editing efficiency may be obtained by performing a method including: preparing a plasmid library including oligonucleotides including pegRNA-encoding nucleotide sequences, and target nucleotide sequences targeted by the pegRNA; introducing the plasmid library and a prime editor into cells; performing deep sequencing on DNA obtained from the cells; and analyzing prime editing efficiency from data obtained through deep sequencing. Herein, details about the cell types and prime editor types are as described above.

The dataset on prime editing efficiency may include information on pairs of pegRNA-encoding sequences and target sequences for all edit types of 1-nt to 3-nt length.

The edit types may include substitution, insertion, and deletion.

The dataset on prime editing efficiency may include information on pairs of pegRNA sequences that cause edits such as substitutions, insertions, or deletions of 1-nt, 2-nt, or 3-nt length and of their corresponding target sequences, and the dataset on prime editing efficiency may include information on prime editing efficiency, which is induced by the each of the pegRNAs depending on the cell types and prime editor types.

A length of reverse transcription template (RTT) of the pegRNAs may be up to 50-nt but is not limited thereto.

A length of a primer binding site (PBS) of the pegRNAs may be between 1-nt and 17-nt but is not limited thereto.

The plasmid library may be prepared by steps including designing pegRNAs that differ in PBS length, RTT length, edit location, edit type, whether PAM (protospacer adjacent motif) co-editing occurs, and the number of nucleotides to be edited; and preparing oligonucleotides including pairs of nucleotide sequences encoding each of the designed pegRNAs and their target nucleotide sequences targeted by the each of the pegRNAs.

The edit locations may be calculated based on a nicking site. For example, the edit locations may be expressed as +1, +2, and +3 from the nicking site.

The term “nicking site” refers to a site cleaved by a Cas9-nickase in a target sequence.

The term “reverse transcriptase (RT)” refers to an enzyme that uses RNA as a template and synthesizes a new complementary DNA.

The term “prime editing guide RNA (pegRNA)” includes a guide sequence that recognizes a target sequence, a tracrRNA scaffold sequence, a primer binding site (PBS) required to initiate reverse transcription, and a reverse transcriptase template (RTT) containing desired genetic changes.

The guide sequence in the pegRNA includes a sequence that is fully or partially complementary to its target sequence.

The term “target sequence” refers to a target nucleotide sequence targeted by a pegRNA. The target sequence may be a sequence expected to be targeted by the pegRNA. The target sequence may be a partial sequence among known genome sequences or may be an arbitrarily designed sequence that a user is willing to analyze using the model of the present disclosure. The predictive model on prime editing efficiency trained by the method according to one aspect may be used for various genetic diseases because there are no limitations in its applicable target region.

The term “oligonucleotide” refers to a substance in which several to hundreds of nucleotides are linked by phosphodiester bonds. The length of the oligonucleotide may be 100 nts to 300 nts, 100 nts to 250 nts, or 100 nts to 200 nts, but is not limited thereto. The length may be appropriately adjusted by those skilled in the art.

The pegRNA-encoding nucleotide sequence included in the oligonucleotide may include a guide sequence, an RTT sequence, and a PBS sequence.

The target nucleotide sequences included in the oligonucleotides may include a protospacer adjacent motif (PAM) and an RTT binding site. The RTT binding site may include a sequence that is fully or partially complementary to the RTT sequence.

The oligonucleotide may further include a barcode sequence. Accordingly, the oligonucleotides may include a pegRNA-encoding sequence, a barcode sequence, and a target sequence targeted by the pegRNA. The number of the barcode sequence may be 1, 2, or more. The barcode sequence may be appropriately designed by those skilled in the art based for the purpose. For example, the barcode sequence may allow a pair of each of pegRNAs and its corresponding target sequence to be identified after deep sequencing.

The oligonucleotide may further include an additional sequence to which a primer may be bound so that the oligonucleotide may be PCR amplified.

The term “library” refers to a group (pool or population) containing two or more types of substances of the same type with different features. Thus, an oligonucleotide library may be a population containing two or more oligonucleotides with different nucleotide sequences, for example, pegRNA, and/or two or more oligonucleotides with different target sequences. Additionally, a plasmid library may be a group of two or more plasmids with different features, for example, a group of plasmids containing different types of oligonucleotides.

The term “vector” may refer to a vehicle that may deliver the oligonucleotide or prime editor into a cell. Specifically, the vector may include each of oligonucleotides containing the pegRNA-encoding sequence and the target sequence. Additionally, the vector may include a prime editor. The vector may be a viral vector or a plasmid vector but is not limited thereto. The viral vector may be a lentiviral vector or a retroviral vector, but is not limited thereto. When the vector is present in a cell of an individual, the vector may contain essential regulatory elements operably linked to an insert (e.g., oligonucleotide or prime editor) so that the insert may be expressed. The vector may be prepared and purified using standard recombinant DNA techniques. Vector types are not particularly limited as long as a vector may function in a target cell such as prokaryotic cells and eukaryotic cells. The vector may include a promoter, start codon, and stop codon terminator. In addition, the vector may appropriately contain DNA encoding a signal peptide, and/or an enhancer sequence, and/or an untranslated region on the 5′ and 3′ sides of the desired gene, and/or a selection marker site, and/or a replicable unit.

The method of delivering a vector to a cell may be achieved using various methods known in the art. For example, delivering the vector to a cell may be performed by several methods known in the art, including a calcium phosphate-DNA coprecipitation method, DEAE-dextran-mediated transfection method, polybrene-mediated transfection method, electric shock method, microinjection method, liposome fusion method, and lipofectamine and protoplast fusion method. Additionally, when using a viral vector, a target may be delivered into a cell using viral particles through infection. In addition, the vector may be introduced into a cell by gene bombardment or the like. The introduced vector may exist as a vector within the cell or may be integrated into a chromosome, but is not limited thereto.

Types of a cell, into which the vector may be introduced, may be appropriately selected by those skilled in the art depending on the vector types and/or the type of a target cell. However, the cell types may include bacterial cells such as Escherichia coli, Streptomyces, and Salmonella Typhimurium; yeast cells; fungal cells such as Pichia pastoris; insect cells such as Drozophila and Spodoptera Sf9 cells; animal cells such as Chinese hamster ovary cells (CHO), SP2/0 (mouse myeloma), human lymphoblastoid, COS, NSO (mouse myeloma), 293T, Bow melanoma cells, HT-1080, baby hamster kidney cells (BHK), human embryonic kidney cells (HEK), and PERC.6 (human retina cells); or plant cells. In a still yet further embodiment, the cell types may be selected from HEK293T, HCT116, DLD1, MDA-MB-231, A549, HeLa, and NIH3T3.

The plasmid library prepared herein refers to a group of plasmids containing oligonucleotides containing pegRNA-coding sequences and target sequences. At this time, each plasmid may contain an oligonucleotide with a different pegRNA-encoding sequence and/or target sequence.

To induce prime editing, a prime editor and a plasmid library may be introduced into a cell. The prime editor may refer to a component of a prime editing system including a fusion protein of Cas9 nickase-RT. The prime editor may be introduced into the cell by a vector. The prime editor may be introduced into the cell as the prime editor itself. The method of introducing the prime editor is not limited as long as the prime editor may exhibit activity within the cell. Herein, the description of the vector is the same as described above.

In the cell, prime editing may occur by oligonucleotides containing pairs of pegRNAs and their target sequences, and prime editors. That is, gene editing may occur with respect to the introduced target sequences.

The method of obtaining DNA from a cell, into which the prime editors and plasmid library have been introduced, may be performed using various DNA isolation methods known in the art.

By sequencing the DNA obtained from each cell, the gene editing efficiency in each of the introduced target sequences may be detected. The sequence analysis method is not limited to a specific method as long as data on prime editing efficiency may be obtained, but for example, deep sequencing may be used as the method.

Analyzing prime editing efficiency from the data obtained through deep sequencing may include calculating the prime editing efficiency.

The prime editing efficiency may vary depending on each of the pegRNA sequences, cell types, and prime editor types.

The term “Deep learning” is an artificial intelligence (AI) technique that allows computers to think and train like humans. Deep learning allows machines to learn and solve complex non-linear problems on their own based on artificial neural network theory. Using the deep-learning technique, computers may recognize, infer, and make decisions on their own without a person setting all the criteria, and the deep-learning technique may be widely used for voice, image recognition, and photo analysis. In other words, deep learning may be defined as a set of machine learning algorithms that attempt high-level abstractions (summarizing key content or functions in large amounts of data or complex data) through a combination of several nonlinear transformation techniques.

In the training method according to the aspect, the deep learning may be performed based on a convolutional neural network (CNN) equipped with a gated recurrent unit (GRU), but the performing is not limited thereto.

In one example, a deep learning-based computation model was developed to predict prime editing efficiency in each of all target genes, which were induced by each of the pegRNAs with variable PBS and RTT lengths designed to introduce 1-nt to 3-nt substitutions, insertions, or deletions at locations +1 to +30. The deep learning-based computation model was named DeepPrime. Additionally, DeepPrime-FT was developed by fine-tuning the DeepPrime with 18 datasets on prime-editing efficiency, derived by each of 8 different prime editing systems containing two types of scaffold sequences in 7 different cell types. Using the DeepPrime-FT, it is possible to predict prime editing efficiency for all possible edit types involving up to three base pairs across various cell types and different kinds of prime editors.

Another aspect provides a method for predicting prime editing efficiency. The method may be a method for predicting prime editing efficiency using a predictive model for prime editing efficiency trained by the training method according to the aspect.

Specifically, a predictive model for prime editing efficiency of each of pegRNAs for various cell types and various types of prime editors is provided. More specifically, a predictive model for the prime editing efficiency of each of pegRNAs for all edit types of up to 3-nt length in various cell types and various types of prime editors is provided. Even more specifically, a predictive model for the prime editing efficiency of each of pegRNAs for all edit types of 1-nt to 3-nt length in 7 cell types and 8 prime editing types is provided.

The method includes obtaining information on cell types, prime editor types, and target sequences; and predicting prime editing efficiency of each of pegRNAs by applying the information to a predictive model for prime editing efficiency trained by the training method according to the aspect.

The input information includes cell types, prime editor types, and target sequences.

Details of the cell types and prime editor types are as described above.

The cell types and prime editor types may be appropriately selected depending on the user's desired experimental conditions. For example, the cell types and prime editor types may be appropriately selected to match the experimental conditions the user wants to check.

The target sequences refers to target nucleotide sequences of pegRNAs that are intended for analyzing or predicting prime editing efficiency. The target sequences may be derived from a genome sequence of the individual that is intended for confirming prime editing efficiency. Alternatively, the target sequences may be any sequences designed and synthesized by methods known in the art. However, the types of the target sequences are not limited as long as the target sequences are sequences that can be applied to the method of the present disclosure for predicting prime editing efficiency. Since the application of the target sequences are not limited to a specific region, the method can be used for various genetic diseases.

The information on the target sequences may include pairs of unedited sequences (i.e., sequences before editing) and edited sequences (i.e., sequences after editing). The unedited sequences may be wild-type (WT) sequences. The edited sequences may be sequences into which intended editing has been introduced by the pegRNAs.

The length of the target sequences may be 10-nt to 150-nt, 20-nt to 150-nt, 30-nt to 150-nt, 10-nt to 130-nt, 20-nt to 130-nt, 30-nt to 130-nt, 40-nt to 130-nt, 50-nt to 130-nt, 10-nt to 100-nt, 20-nt to 100-nt, 30-nt to 100-nt, 40-nt to 100-nt, or 50-nt to 100-nt, but is not limited thereto.

The target sequences may include, but are not limited to, a protospacer adjacent motif (PAM), and a protospacer sequence. The PAM and the protospacer sequence are sequences involved in the process of Prime Editor recognizing the target sequence.

The information may further include information about edit lengths and edit types.

The edit lengths may be any one selected from the group consisting of 1-nt, 2-nt, and 3-nt.

The edit types may be any one selected from the group consisting of substitution, insertion, and deletion.

The information may further include information on an RTT length of a pegRNA and a PBS length of a pegRNA.

The information on the RTT length of the pegRNA may be a maximum RTT length. The maximum RTT length may be selected from any length of 50-nt or less but is not limited thereto.

The PBS length of the pegRNA may be a minimum PBS length and a maximum PBS length. The minimum and maximum PBS lengths may be selected from any length ranging from 1-nt to 17-nt, but are not limited thereto.

In the prediction, the prime editing efficiency of each of pegRNA may be predicted depending on the cell types and prime editor types.

The method may further include outputting the pegRNA sequences and the predicted prime editing prediction scores for the pegRNA sequences.

The output prime editing prediction scores may be expressed as values calculated for prime editing efficiency, or as relative values to a preset reference value. However, the form or type of information on the output prime editing prediction scores is not limited.

The output pegRNA sequences may include a guide sequence, a PBS sequence, and an RTT sequence.

The output may be an output obtained by sorting the pegRNAs in order of highest prime editing prediction scores.

According to the method for predicting prime editing efficiency according to the aspect, pegRNA sequences with high prime editing efficiency may be selected for various cell types and various prime editor types. Therefore, it is possible to select prime editor types and pegRNA sequences that exhibit high prime editing efficiency in a specific cell type without individual experiments.

A further aspect provides an apparatus for predicting prime editing efficiency. The apparatus may be an apparatus for predicting prime editing efficiency using the predictive model for prime editing efficiency trained by the training method according to the aspect. The apparatus may be an apparatus that implements the method for predicting prime editing efficiency according to the aspect. Accordingly, the descriptions related to the method for training a predictive model for the prime editing efficiency, the predictive model for the prime editing efficiency, and the method for predicting prime editing efficiency may be equally applied.

The apparatus may include an input unit configured to receive information on cell types, prime editor types, and target sequences; and a prediction unit configured to apply the information to predictive models, which are trained according to the method of the aspect to predict the prime editing efficiency of each of the pegRNAs.

The information on the target sequences may include pairs of unedited sequences and edited sequences.

The information may further include information on edit lengths and edit types.

The method may further include an output unit configured to output pegRNA sequences and prime editing prediction scores predicted for the pegRNAs.

The output unit may output pegRNA sequences in order of highest prime editing prediction scores.

The output pegRNA sequences may include a guide sequence, a PBS sequence, and an RTT sequence.

A yet further aspect provides a computer-readable recording medium on which a program is recorded, the program being configured to cause a computer to execute the method for predicting an off-target prime editing efficiency according to one aspect.

The program may be an implementation of the predictive model for prime editing efficiency or the method for predicting prime editing efficiency in computer programming languages.

The computer programming languages that can be used to implement the program include but are not limited to, Python, C, C++, Java, Fortran, and Visual Basic. The program may be stored in a recording medium such as USB memory, a compact disc read only memory (CDROM), a hard disk, a magnetic diskette, or a similar media or device. The program may be connected to an internal or external network system. For example, a computer system may use HTTP, HTTPS, or XML protocols to access sequence databases such as NCBI GenBank to search nucleic acid sequences of target genes and regulatory regions of the genes.

The program may be provided online or offline.

A still yet further aspect provides a method for training a predictive model for off-target prime editing efficiency. Specifically, a method for training a predictive model for prime editing efficiency in each of off-target sequences is provided.

The term “off-target prime editing efficiency” refers to the efficiency of editing genes at unwanted locations by each of prime editors and pegRNAs. The occurrence of off-target prime editing may reduce safety of gene scissors. Therefore, by predicting the off-target prime editing efficiencies of the pegRNAs, it is possible to select a pegRNA with no or very low probability of off-target prime editing occurring. The selected pegRNA may be expected to have a high safety.

The method includes obtaining a dataset on the prime editing efficiencies of the pegRNAs on on-target sequences and off-target sequences; and training the predictive model using the dataset by deep learning, to establish relationships between a feature affecting an off-target prime editing and the off-target prime editing efficiency.

A prime editing efficiency of a pegRNA in an on-target sequence may mean a ratio at which the peg-RNA-induced editing occurred in a sequence targeted by the pegRNA.

A prime editing efficiency of a pegRNA in an off-target sequence may refer to a ratio at which pegRNA-induced editing occurs in a sequence non-targeted by the pegRNA.

The off-target prime editing efficiency may be prime editing efficiency induced by a pegRNA in the off-target sequence. The off-target prime-editing efficiency may be a relative editing efficiency obtained by dividing the prime-editing efficiency induced by the pegRNA in the off-target sequence by the prime-editing efficiency induced by the pegRNA in the on-target sequence.

In one embodiment, the dataset may further include a dataset used in the method of training a predictive model for prime editing efficiency according to the aspect. That is, the dataset may include a dataset on the prime editing efficiency of the pegRNA in an on-target sequence and off-target sequence depending on cell types and prime editor types. Accordingly, a predictive model for off-target prime editing efficiency of a pegRNA depending on combination of various cell types and various prime editor types may be prepared.

The dataset on prime editing efficiency may be obtained by performing a method including: preparing a plasmid library containing oligonucleotides including pegRNA-encoding nucleotide sequences, and either an on-target or an off-target nucleotide sequence; introducing the plasmid library and prime editors into cells; performing deep sequencing on DNA obtained from the cells; and analyzing prime editing efficiency from data obtained through deep sequencing.

The dataset on prime editing efficiency may include information on pairs of pegRNA-encoding sequences and their target sequences for all edit types of 1-nt to 3-nt length.

The edit types may include substitution, insertion, and deletion.

The target sequence may include an on-target sequence and an off-target sequence.

The dataset on prime editing efficiency may include information on pairs of the pegRNA sequences that cause edits such as substitutions, insertions, or deletions of 1-nt, 2-nt, or 3-nt length and their target sequences (on-target sequences or off-target sequences), and the dataset on prime editing efficiency may include information on prime editing efficiency induced by each of the pegRNAs.

The plasmid library may be prepared by steps including designing pegRNA-target sequence pairs with variations in locations of the mismatch (off-target), RTT lengths, PBS lengths, edit types, and number of mismatches; and preparing oligonucleotides containing pairs of nucleotide sequences encoding each of the designed pegRNAs and their corresponding target nucleotide sequences.

The locations of the mismatch may refer to the locations of mismatch among locations of the target sequences that interact with the pegRNAs.

The number of mismatches may be 1 to 10 or 1 to 6.

The features affecting the off-target prime editing may be obtained from information on elements involved in the off-target prime editing. The features affecting the off-target prime editing may be features obtained by analyzing data on the off-target prime editing efficiency. The features affecting the off-target prime editing efficiency may be obtained by the predictive model for off-target prime editing efficiency. Features obtained by performing a separate method can also be used.

In another embodiment, the features affecting the off-target prime editing may include one or more selected from the group consisting of a location of the mismatch, the number of the mismatch, types of the mismatch, a length of a primer binding site (PBS) of a pegRNA, and a length of a reverse transcription template (RTT), but are not limited thereto.

In the types of the mismatch, the mismatch (off-target) may include a mismatch that causes purine-pyrimidine base pairing, a mismatch that causes purine-purine base pairing, and a mismatch that causes pyrimidine-pyrimidine pairing.

In a further embodiment, DeepPrime was fine-tuned using a dataset on prime editing efficiency of 47,839 pairs of pegRNAs, and their on-target and off-target sequences. As a result, DeepPrime-Off was developed. The DeepPrime-Off further inputted sequence information on pairs of the off-target sequence and pegRNA sequences, considering the interaction between them. When using the DeepPrime-Off, the prime editing efficiency induced by a pegRNA in the off-target sequences may be predicted.

A still yet further aspect provides a method for off-target prime editing efficiency.

The method may include obtaining information on target sequence and pegRNA sequences; and
predicting off-target prime editing efficiency of each of pegRNAs by applying the information to a predictive model for off-target prime editing efficiency trained by the training method according to the aspect.

The information on the target sequences may include off-target sequences.

The method may further include outputting predicted off-target prime editing prediction scores for the pegRNAs.

A still yet further aspect provides an apparatus for off-target prime editing efficiency.

The apparatus may include an input unit configured to receive information on target sequences and pegRNA sequences; and

a prediction unit configured to apply the information to the predictive model, which is trained according to the method of one aspect to predict the off-target prime editing efficiency of each of the pegRNAs.

The information on the target sequence may include an off-target sequences.

The apparatus may further include an output unit configured to output predicted prime editing prediction scores for the pegRNAs.

A yet further aspect provides a computer-readable recording medium on which a program is recorded, the program being configured to cause a computer to execute the method for predicting an off-target prime editing efficiency according to one aspect.

Redundant content is omitted considering the complexity of this specification. Terms not otherwise defined in this specification have meanings commonly used in the technical field to which the present disclosure pertains.

### Advantageous Effects

According to one aspect, a method of training predictive model for prime editing efficiency, and a method and apparatus for predicting prime editing efficiency using the same enable prediction of prime editing efficiency for various prime editor types in various cell types. In addition, since the method and apparatus for predicting prime editing efficiency have no limitations on their applicable target region, they can be used for various genetic diseases.

According to another aspect, a method of training predictive model for off-target prime editing efficiency, and a method and apparatus for predicting prime editing efficiency using the same enable prediction of prime editing efficiency in the off-targets.

Using the methods and apparatuses, prime editing efficiency is predicted on on-target and/or off-target sequences for various combinations of the prime editors and pegRNAs in various cell types. Thereby, the most efficient and specific combination of a prime editor and a pegRNA can be selected. Therefore, the methods and apparatuses can be useful in all fields where gene scissors are applied, such as disease treatment by gene editing.

### DESCRIPTION OF DRAWINGS

FIG. 1. High-throughput Profiling of PE2 Efficiency

(A) Schematic representation of a pairwise library structure used in this study, hU6, human U6 promoter; pT, poly T sequence, see also FIG. 8A, (B, C) Heat maps showing mean efficiency with PBS of all possible lengths (1-17 nts) (B) or RTT of various lengths (5-35 nts) (C), see also FIG. 9A, (D) Effect of locations of edits on PE2 efficiency when a 1-bp substitution is introduced, (E-G) Effects of right homology arm (RHA) lengths on prime editing efficiency, see also FIG. 9C, (H) Effect of edited base pair number on prime editing efficiency, error bars represent 95% confidence intervals, (I-J) Line plots showing mean prime editing efficiency when insertions (I) or deletions (J) of different lengths are introduced, (K) Prime editing efficiency induced by each of pegRNAs with different nucleotides at a final template location, herein the top, middle, and bottom lines in the box represent the 25th, 50th, and 75th percentiles, respectively, and whiskers represent minimum and maximum values, and crosses in the box plot indicate mean prime editing efficiency, number of pegRNA and target sequence pairs n=70,911 (cytosine; C), 66,321 (thymine; T), 66,589 (adenine; A), and 84,972 (guanine; G), see also FIGS. 9D and 9F, (H, K) Subsets of experimental groups (P<0.05, ANOVA followed by Türkiye's post hoc test) with no statistically significant difference in prime editing efficiency are indicated by letters a, b, c, d, and e in order of mean prime efficiency,

FIG. 2. Factors Associated with Prime Editing Efficiency

(A) Twenty most important features associated with prime editing efficiency determined by Tree SHAP after accounting for multicollinearity using a threshold of 0.7 for the correlation coefficient, herein factors removed due to multicollinearity are displayed in bold next to the corresponding factors (with ranks without considering multicollinearity are shown in parentheses) or indicated in this legend; i) Number of U in the PBS+RTT region (No. 19), Number of UUs in the PBS+RTT region (No. 37), ii) Tm of target regions corresponding to the RTT region, Tm and reverse transcribed cDNA in the RTT region, iii) RTT length, number of U in the PBS+RTT region, number of UU in the RTT region, iv) Tm of the 74-nt target sequence, GC count within the target sequence, and GC content within the PBS RTT region, and v) Number of CGs in the RTT region, in summary, the violin plots (left graph) show each pair of pegRNAs and target sequences as a dot such that the dot position on the x-axis reflects the SHAP value, a high SHAP value indicates that its corresponding feature is associated with high prime editing efficiency, red or blue dots indicate high or low values of the relevant features, respectively, overlapping points are slightly apart in the y-axis direction, so the density is clear, and Tm represents the melting temperature, number of pegRNA and target sequence pairs (i.e., number of points per feature in summary plot) is n=259, 910, see also FIG. 10A, (B-F) Effect of PBS-associated features (B, C) and RHA-associated features (D-F) on mean prime editing efficiency, and Tm represents the melting temperature, see also FIG. 10B-G, and (G) Effect of locations of edits and edit lengths on mean prime editing efficiency for substitutions (left), insertions (middle), and deletions (right),

FIG. 3. Development of Computation Model for Predicting Prime Editing Efficiency

(A) Schematic representation of a deep learning algorithm used to develop DeepPrime, herein GRU is a gate circulation unit, (B, C) Cross-validations of the machine learning algorithm used to develop a model predicting prime editing efficiency, herein each dot represents Pearson's correlation coefficient (B) or Spearman's correlation coefficient (C) between the measured prime-editing efficiency and the predicted prime-editing efficiency in 5-fold cross-validation, number of correlation coefficients is n=5, statistical comparison of the top two algorithms is shown (two-tailed Steiger test), and bars and error bars represent the mean and standard deviation of Spearman's or Pearson's correlation coefficients, respectively, CNN, convolutional neural network; GRU, gate circulation unit; long short-term memory (LSTM); light gradient boosting machine (LightGBM); extreme gradient boosting (XGBoost); gradient boosting regression (GBR); and random forest (RT), (D-F) DeepPrime evaluations using ClinVar_Test as test dataset, herein each dot color was determined using kernel density estimation with a Gaussian kernel, the dataset ClinVar_Test was split into 9 or 30 data subsets depending on edit types and edit lengths (E) or locations of edits (F), respectively and was used for model evaluation, (F) Presentation of Pearson's and Spearman's correlation coefficients between measured and predicted PE2 efficiencies as solid and dashed lines, respectively, (G, H) Evaluations of DeepPrime using independent test datasets of prime editing efficiency, as a test data set, herein prime editing efficiency measured for each of target sequences (G, Jang et al., 2021) and endogenous regions (H, Liu's initial study on prime editing, Anzalone et al., 2019) combined from previous studies were used, (D, E, G, H) Presentations of Pearson's (r) and Spearman's (R) correlation coefficients and number of pegRNAs (n),

FIG. 4. Improvement of Prime Editing Efficiency

(A) Comparison of prime editing efficiency using a PE2 with pegRNAs containing existing and optimized scaffolds, (B) Effect of PAM co-editing on PE2 efficiency, herein mean prime-editing efficiency obtained through co-editing was PAM normalized with respect to the case without PAM co-editing, (C) Indication of prime editors with the highest mean efficiency on target sequences for each 3-nt PAM sequence in different colors, (D) Heat map showing the maximum average editing efficiency induced by any PE2 variant in the target sequence containing each 3-nt candidate PAM sequence, (E-F) Correlations between prime editing efficiency induced by either PE2 or PE2max (E) and PE4max (F) in targets with NGG PAM sequences, (G-M) Correlations between prime editing efficiency induced by each of PE2, PE2max, PE4max, and NRCH-PE4max in HEK293T cells, and prime editing efficiency on targets with NGG PAM sequences in other cell types, (N-P) Correlations between prime editing efficiency of PE2max and PE4max induced by either pegRNA or epegRNA, and (A, E-M) Presentation of Pearson's (r) and Spearman's (R) correlation coefficients and number (n) of pegRNAs, herein the black dashed line represents y=x,

FIG. 5. Development and Performance of DeepPrime-FT

(A) Schematic diagram of DeepPrime-FT development, (B, C) Heat maps of Pearson's and Spearman's (C) correlation coefficients between predicted and measured prime editing efficiencies showing their dependence on experimental conditions such as cell type, pegRNA scaffold, prime editor, and PAM sequence, herein prime editing using epegRNA is indicated by adding “-e” to the name of the prime editor (for example: Pe2max-e), each box surrounded by a thick line represents the Pearson's or Spearman's correlation coefficients of a model evaluated using a test dataset generated under the same experimental conditions as the training dataset, and the same color gradient is used for both Pearson's and Spearman's correlation coefficients,

FIG. 6. Application of DeepPrime and DeepPrime-FT

(A, B) Predicted data for correction (A) and generation (B) of pathogenic/likely pathogenic mutations reported in ClinVar as the predicted efficiency of pegRNAs designed using DeepPrime, features of highly active pegRNAs, DeepSpCas9 scores, or a rational approach (rational design) based on random selection. Number of pegRNAs per design is n=64, 327, statistical comparison of the top two methods is shown (ANOVA followed by Türkiye's post hoc test). (C) Predicted data for variant introduction into NPC1 and BRCA2 as predicted efficiencies of pegRNAs selected using DeepPrime, DeepPrime-FT and rational design in a previous study (Erwood et al., 2022). Number of pegRNAs per design is n=426. (D) Measured PE efficiency rank distribution of the pegRNA with the highest DeepPrime scores among eight pegRNAs per target. Number of pegRNA sets is n=845. (E) Measured PE efficiency percentile rank distribution of the pegRNA with the highest DeepPrime scores among all tested pegRNAs per target. Number of pegRNA sets is n=9, (D, E) Percentages in parentheses represent the fraction of corresponding ranks (D) or percentile ranks (E) of pegRNAs selected by DeepPrime, (F, G) Comparison of prime editing efficiency predicted and experimentally measured at endogenous regions in BRCA2, number of pegRNA and target sequence pairs tested is (n)=12 (DeepPrime-FT) and 12 (Erwood et al. 2022), herein each measurement is the mean of duplicates, (A-C, F) Presentations of the 25th, 50th, and 75th percentiles by the top, middle, and bottom lines in the boxes, respectively; Presentations of the 10th and 90th percentiles (A-C) or minimum and maximum values (F) by whiskers, (G) Correlation between the predicted prime-editing efficiency of PE2max in an endogenous region and an experimentally measured prime-editing efficiency, (H-J) Correlations between predicted and measured prime editing efficiency of each of PE3 and PE5 in the endogenous region, (G-J) Presentations of Pearson's (r) and Spearman's (R) correlation coefficients and number of pegRNAs and target sequence pairs (n), herein the dotted line represents the trend line,

FIG. 7. High-throughput Evaluation of Prime Editing Efficiency on Off-target Sequences

(A, B) Effect of locations of mismatchs and the number of the mismatchs in the off-target sequences on the efficiency of each of PE2 (A) and Pe2max (B), herein relative editing efficiency was calculated by dividing the prime editing efficiency in the off-target sequences by the on-target efficiency in the on-target sequences, (A) Error bars of 95% confidence intervals, (C) Typical specificity and activity for each of PE2max and PE4max in the cell lines tested, (D) Effect of a PBS length on prime editing efficiency in the off-target sequences, (E, F, G) Effect of a RTT length (E), types of the mismatchs and locations of the mismatchs (F), and intended edit types (G) on prime editing efficiency in the off-target sequences, (H) Schematic diagram of the fine-tuning-based development of DeepPrime-Off, (I, J) Evaluations of DeepPrime-Off, herein data obtained from the combined target sequences (PE2-Off_Test) (I) and the endogenous region (J) were used as test datasets, and Pearson's (r) and Spearman's (R) correlation coefficients and number (n) of pegRNAs are shown, and, in (J), when the measured prime editing efficiency was <0.1% in the off-target region, it was considered a deep sequencing error and no off-target prime-editing activity,

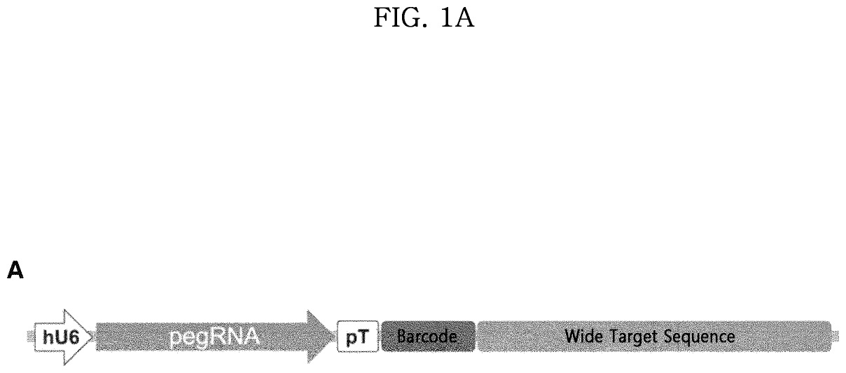

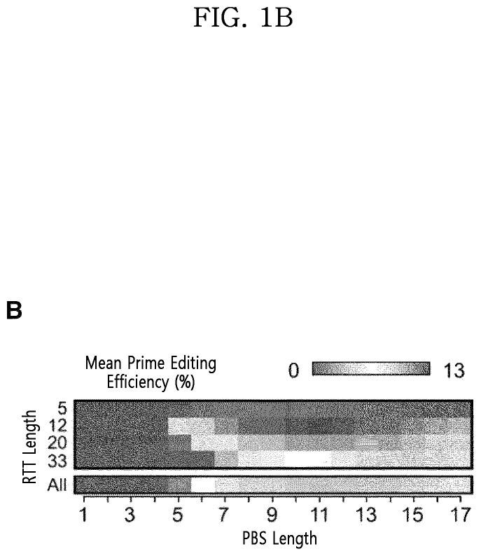

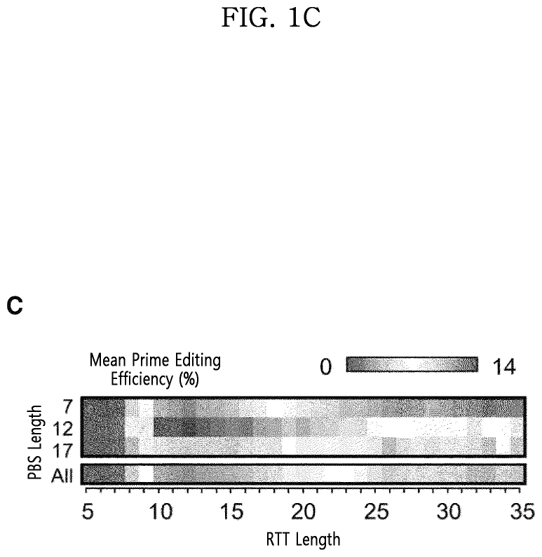

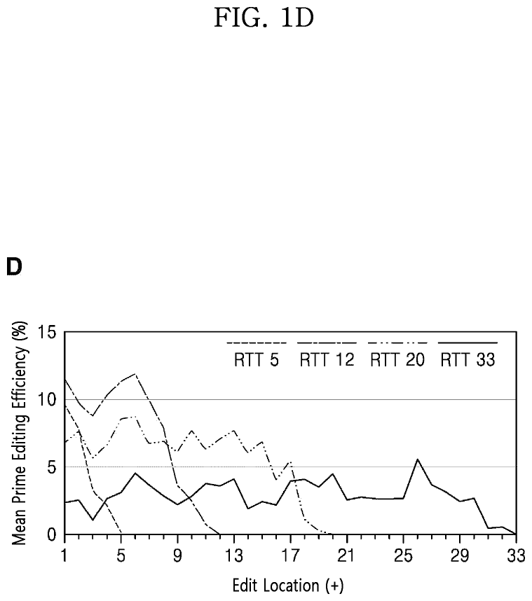

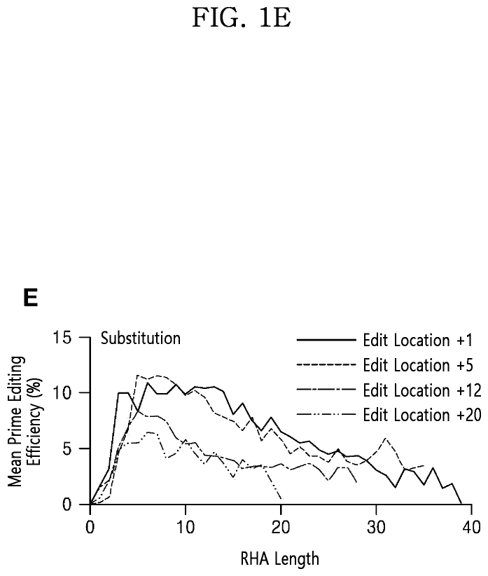

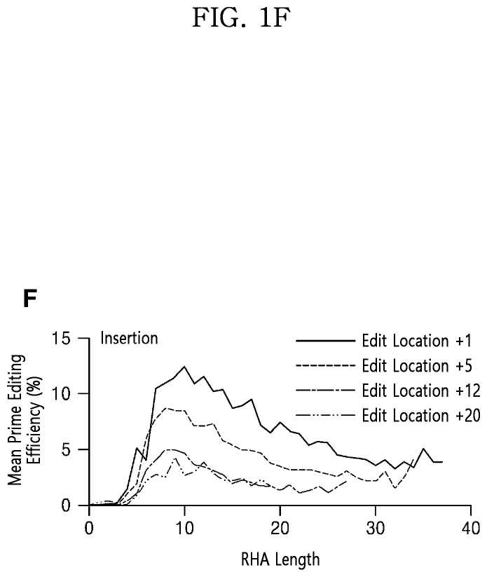

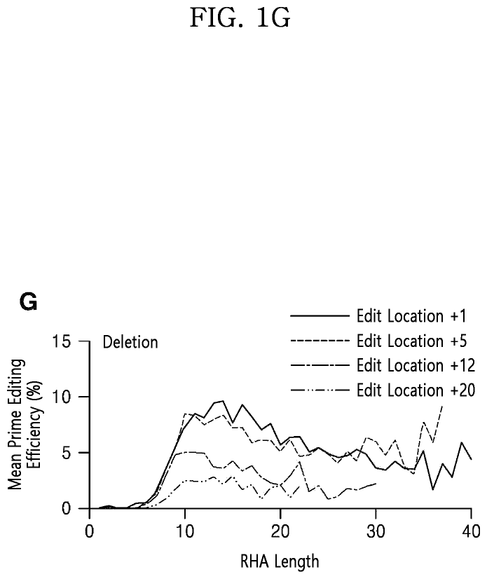

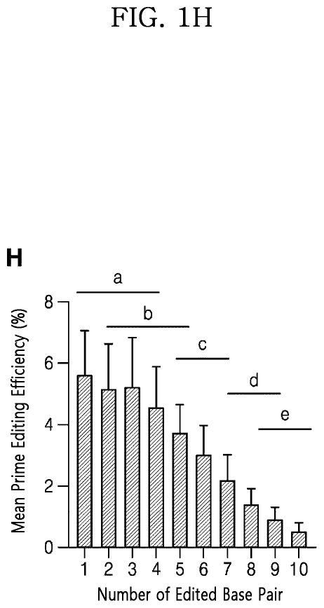

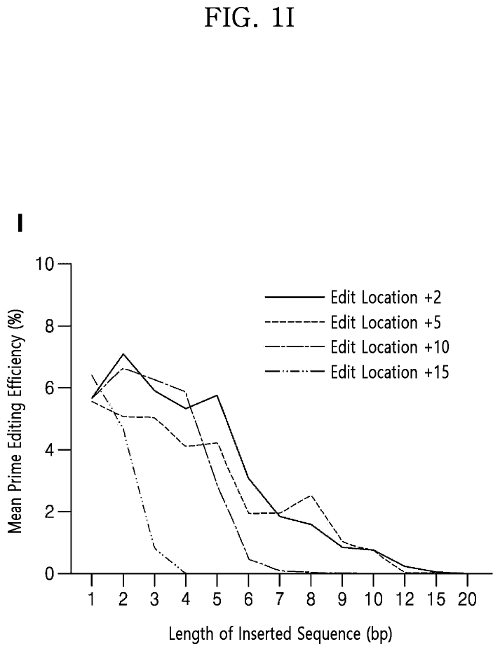

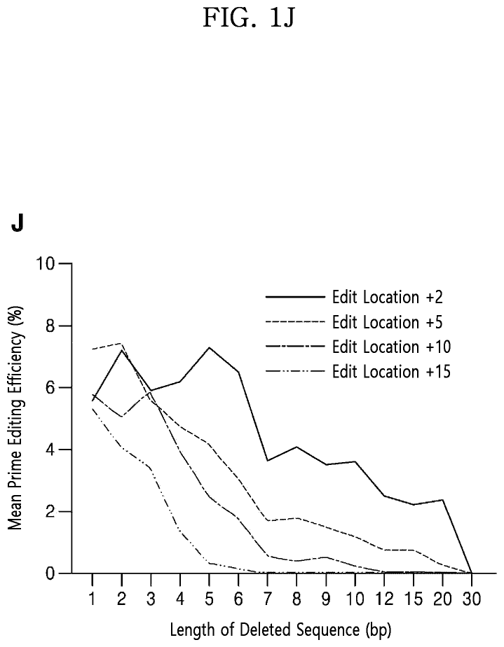

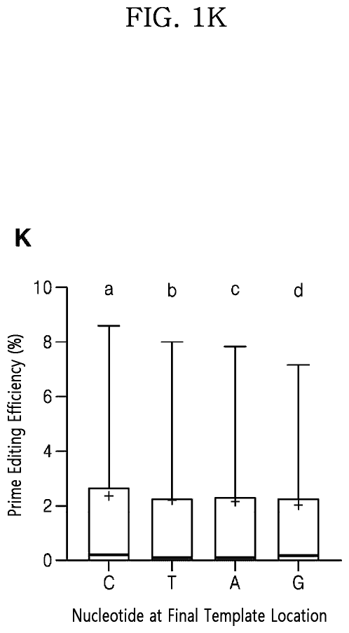

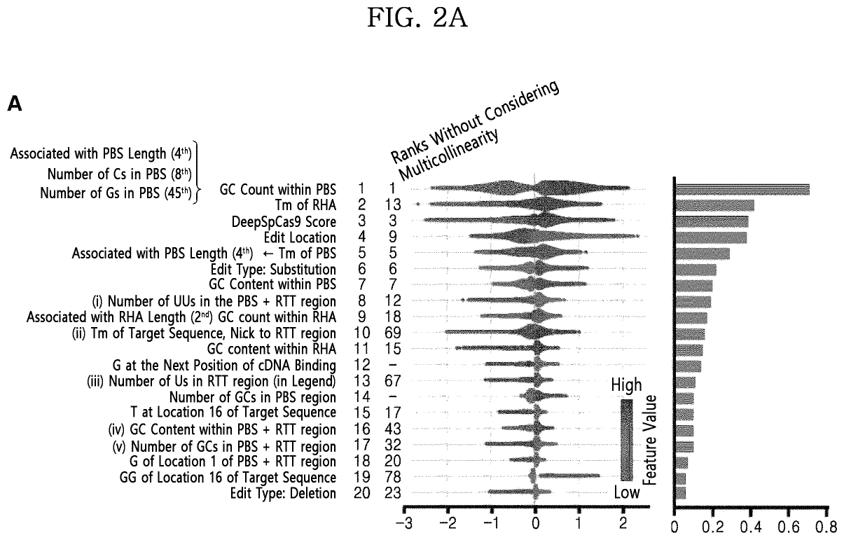

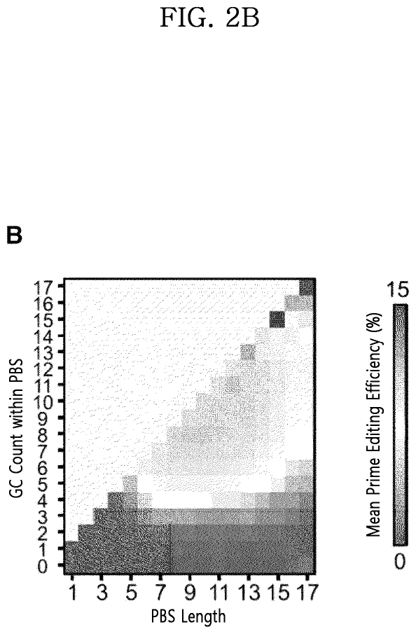

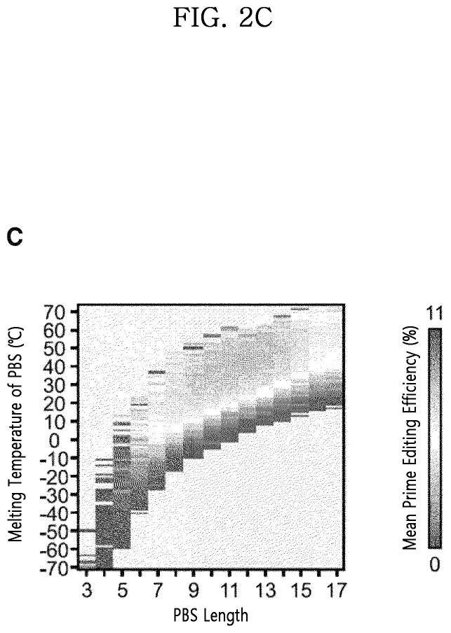

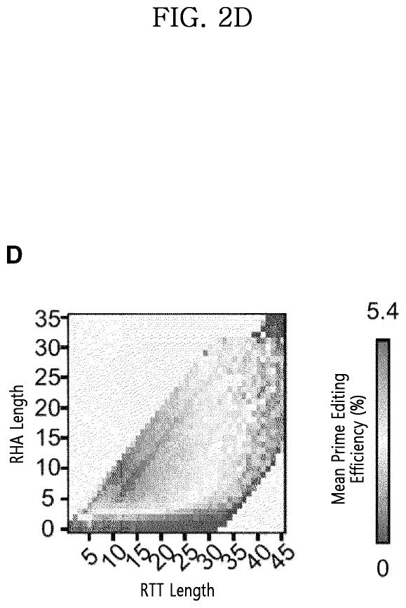

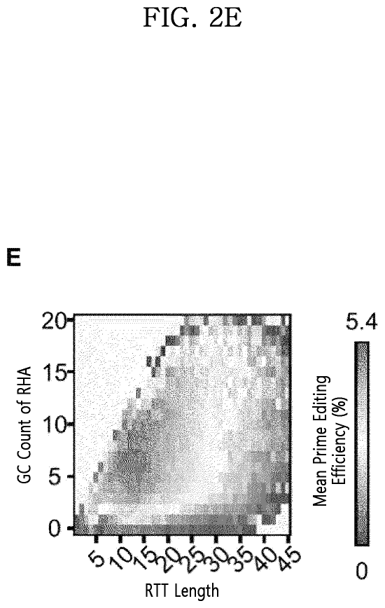

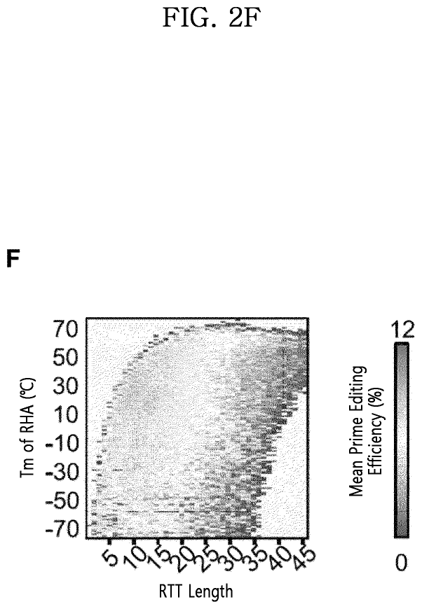

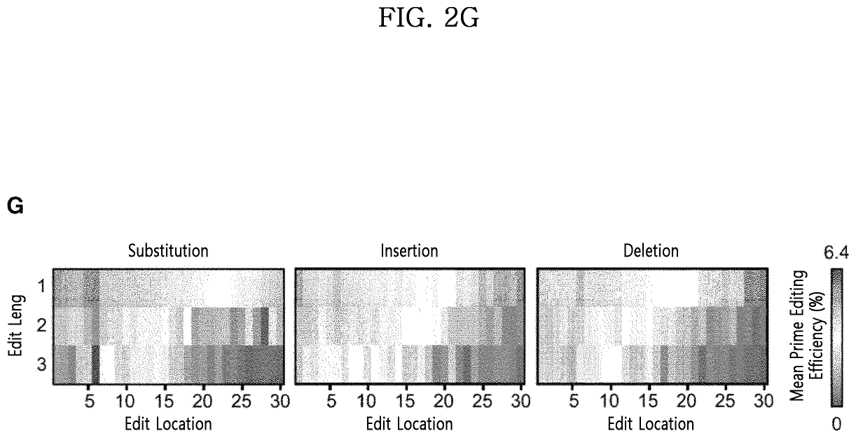

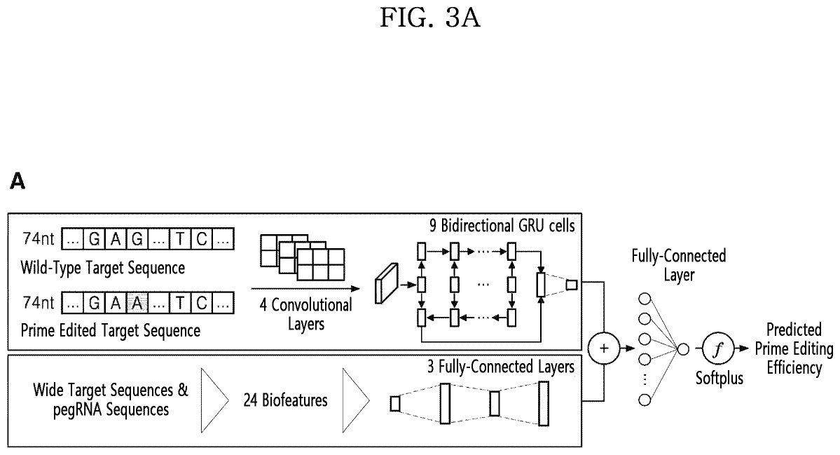

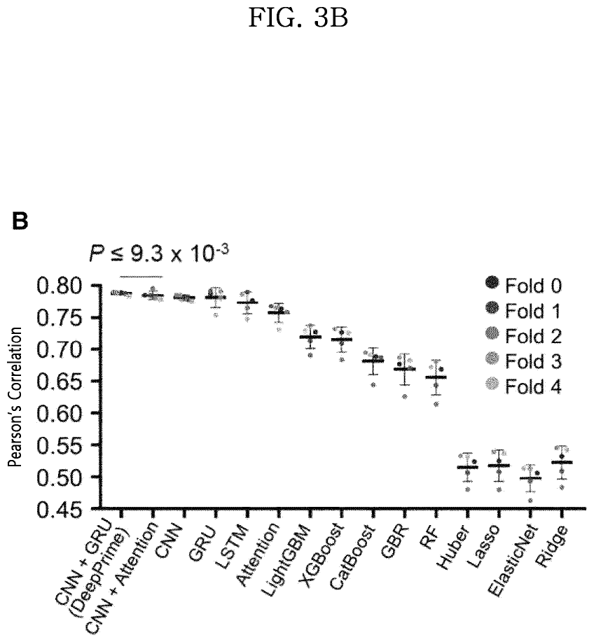

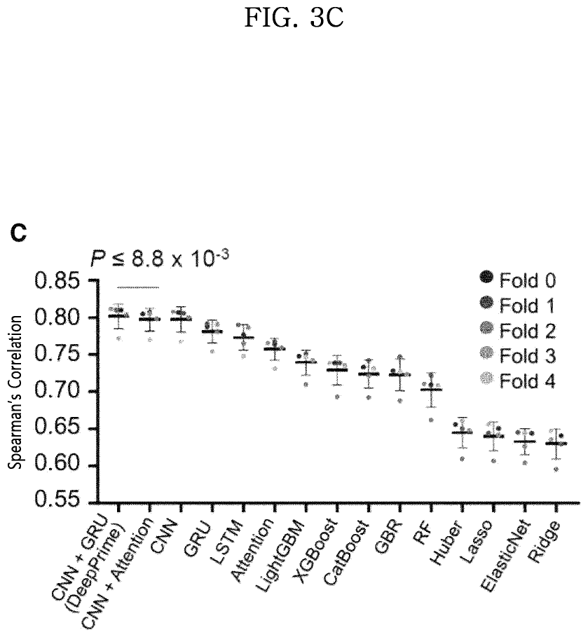

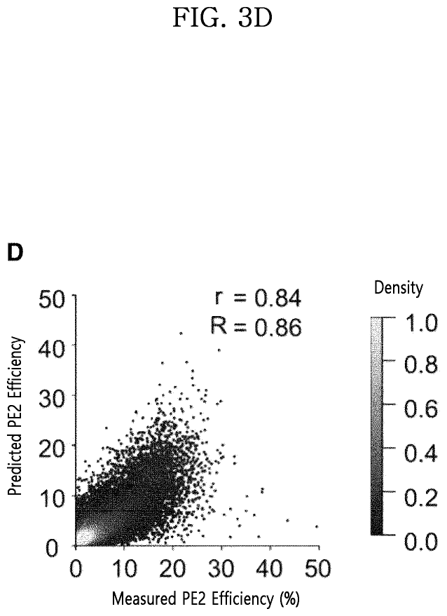

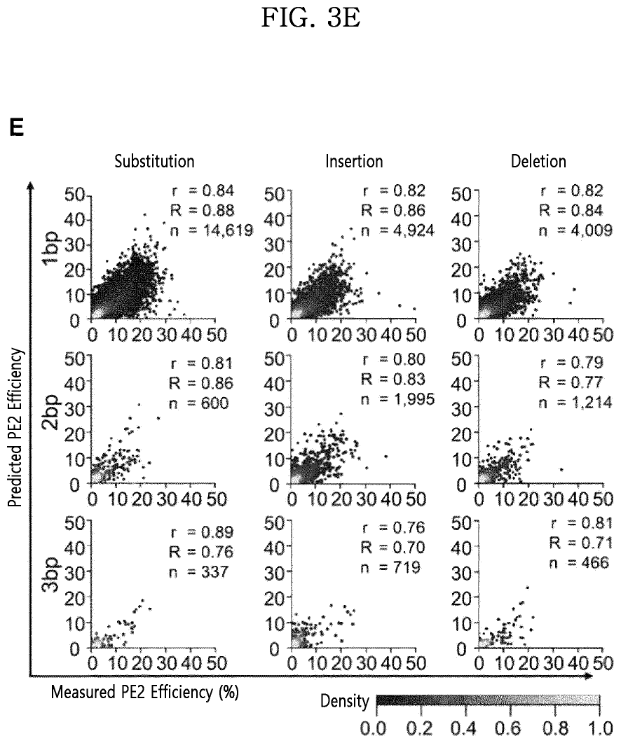

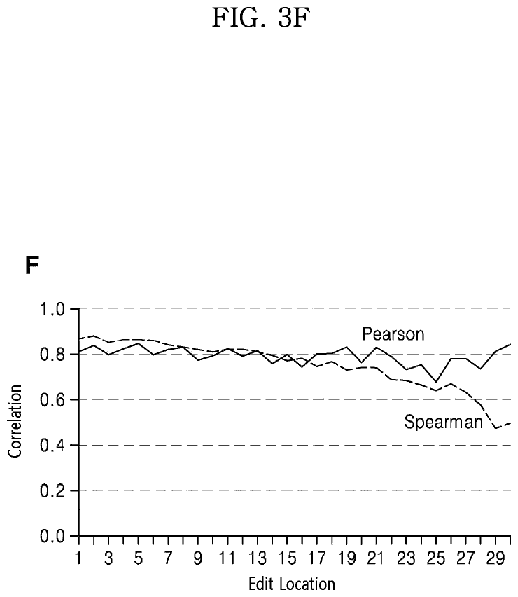

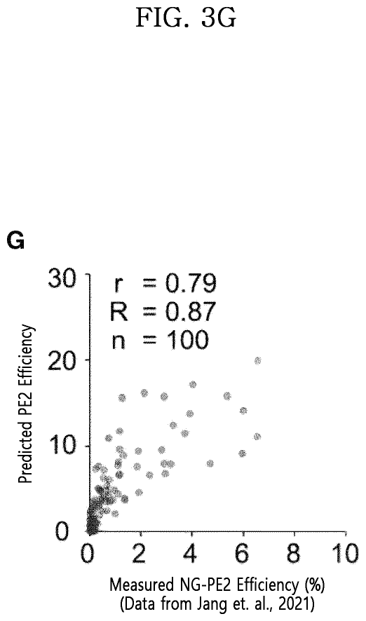

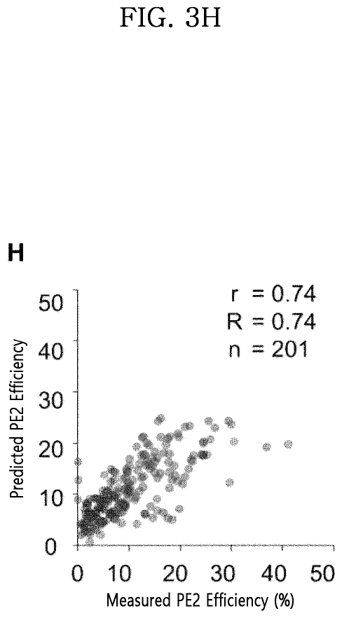

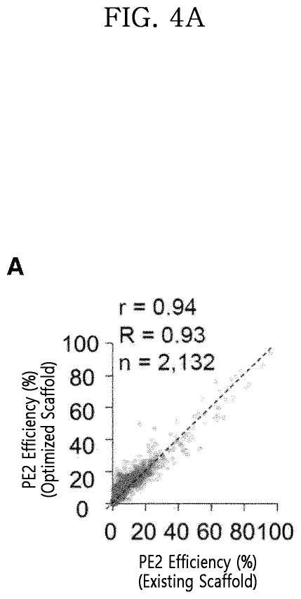

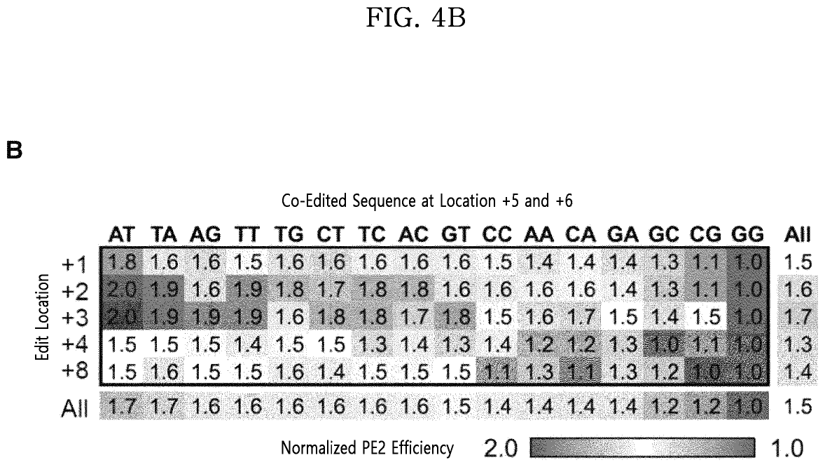

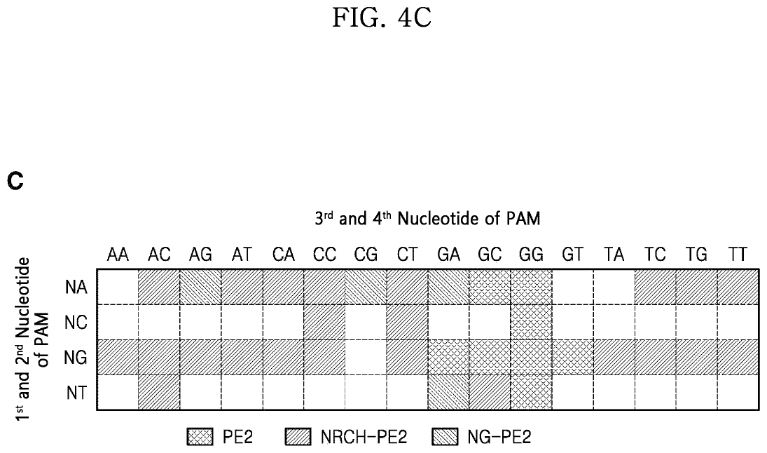

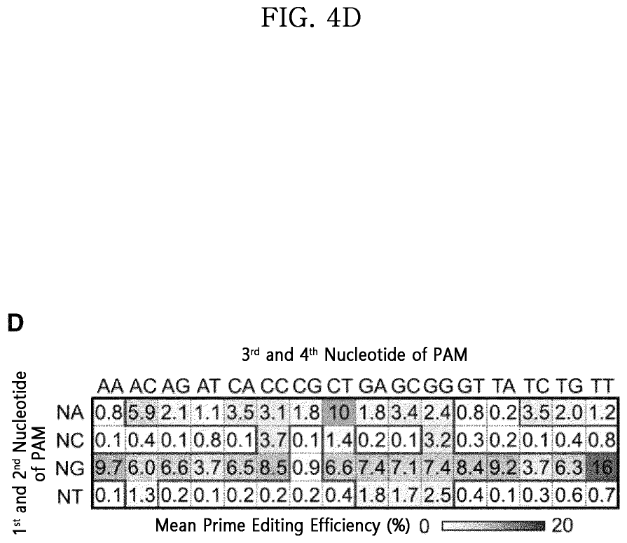

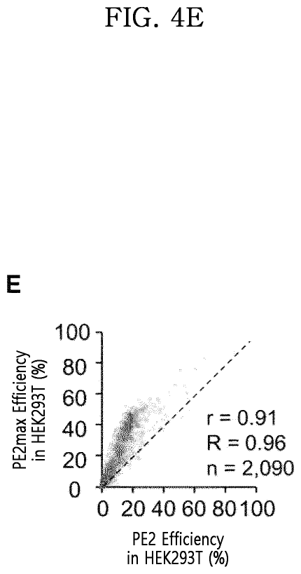

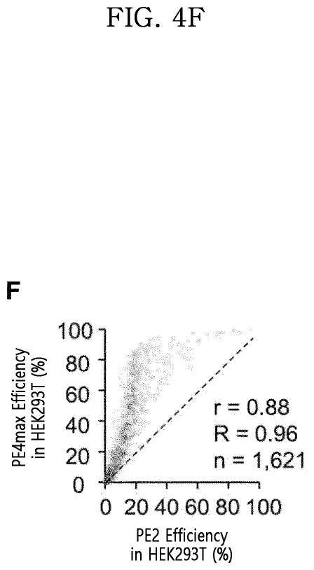

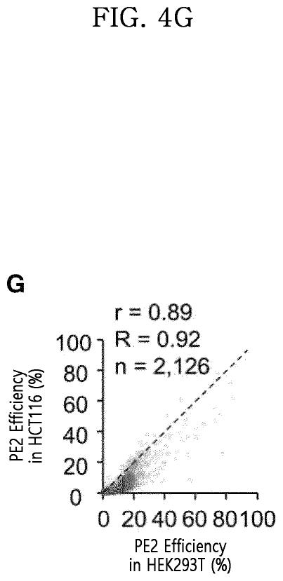

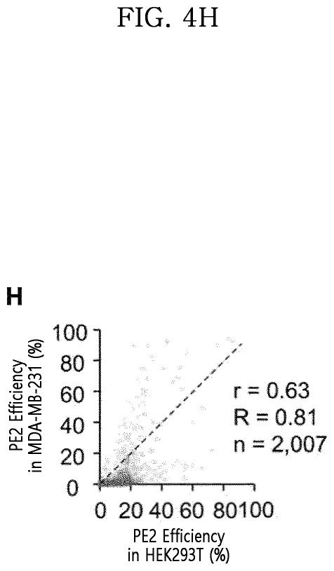

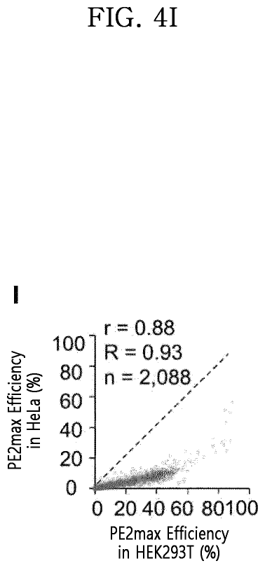

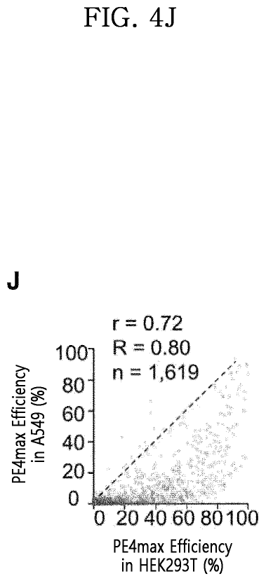

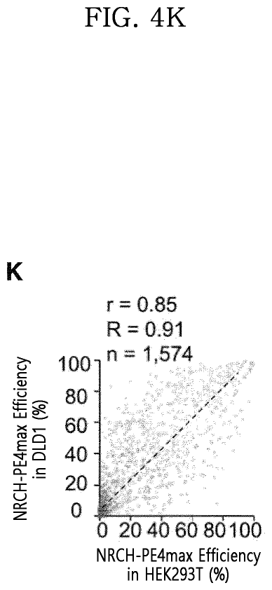

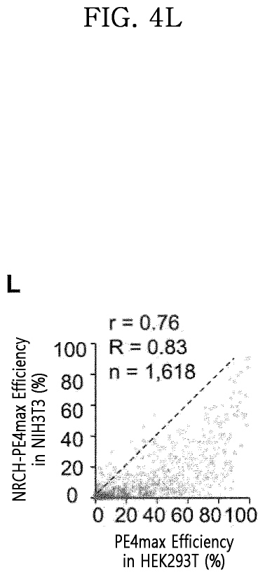

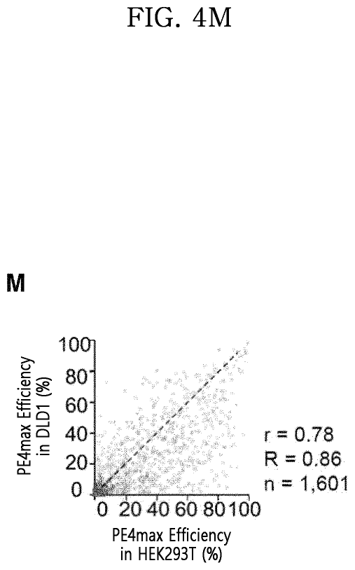

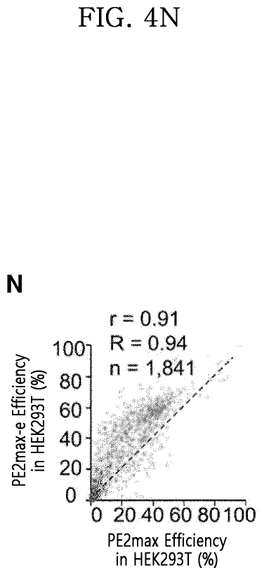

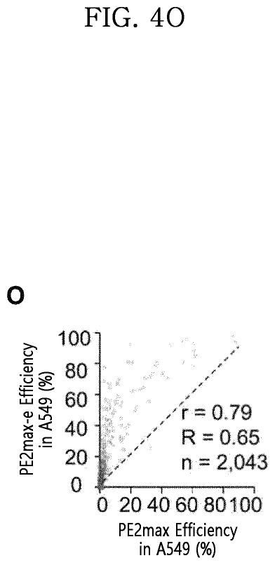

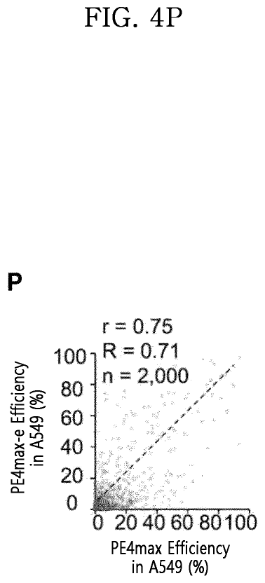

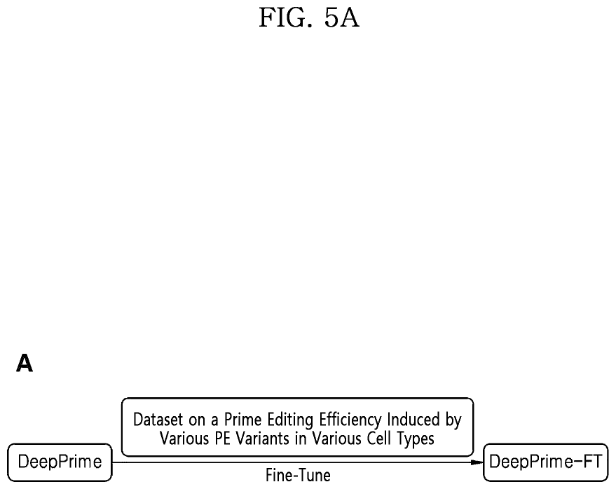

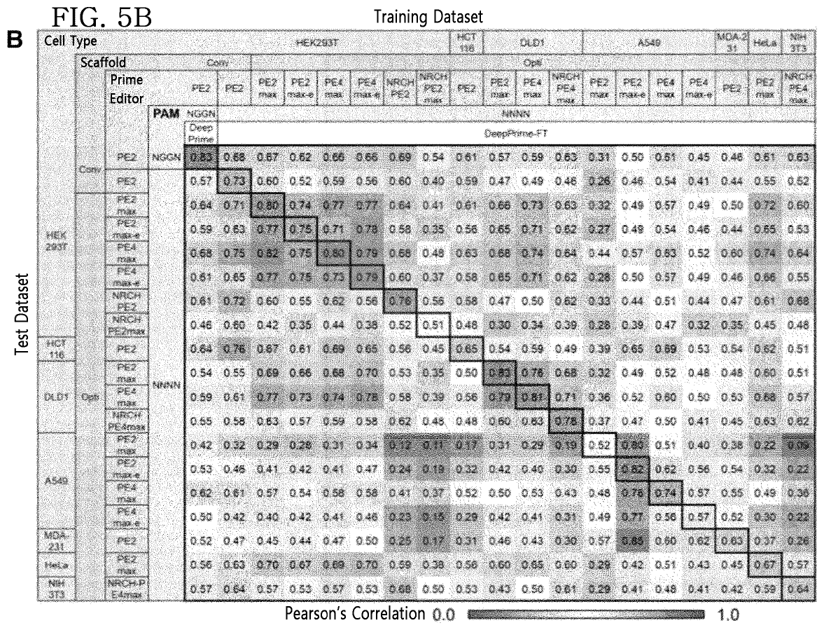

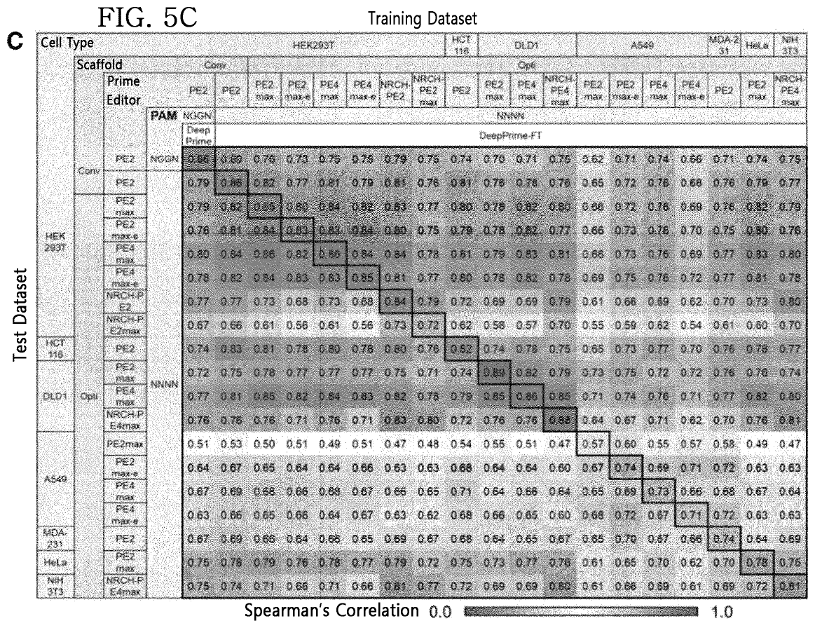

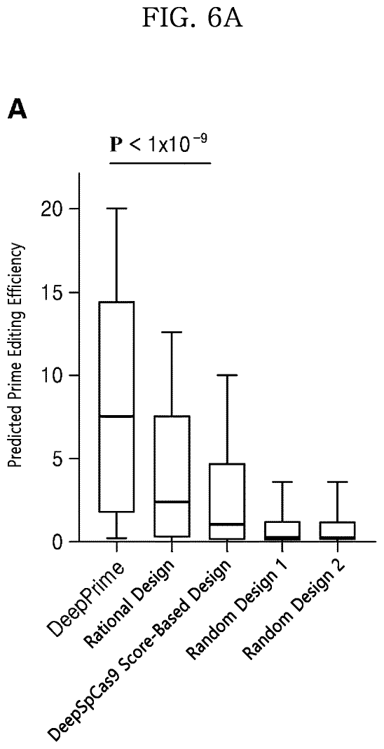

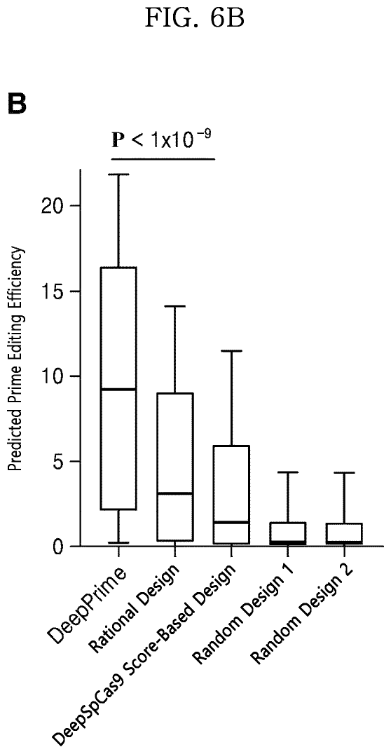

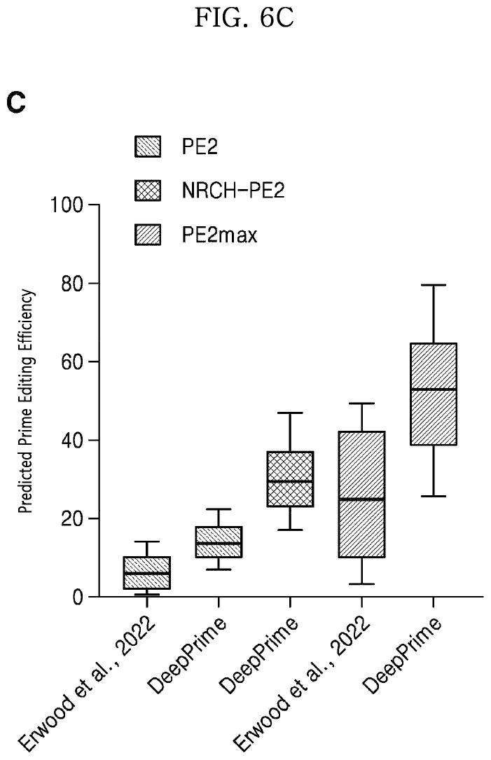

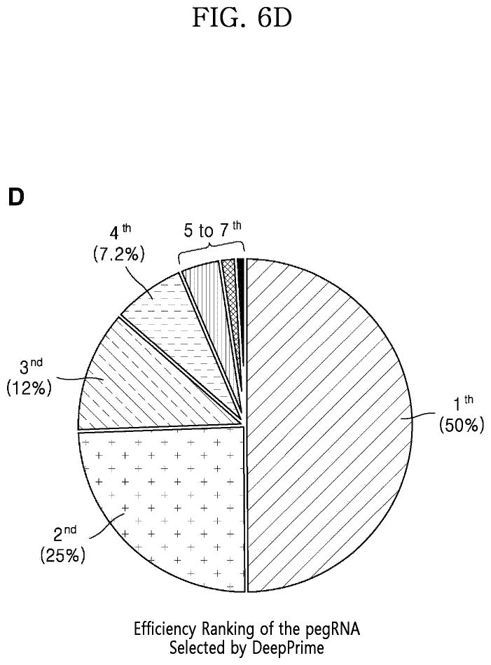

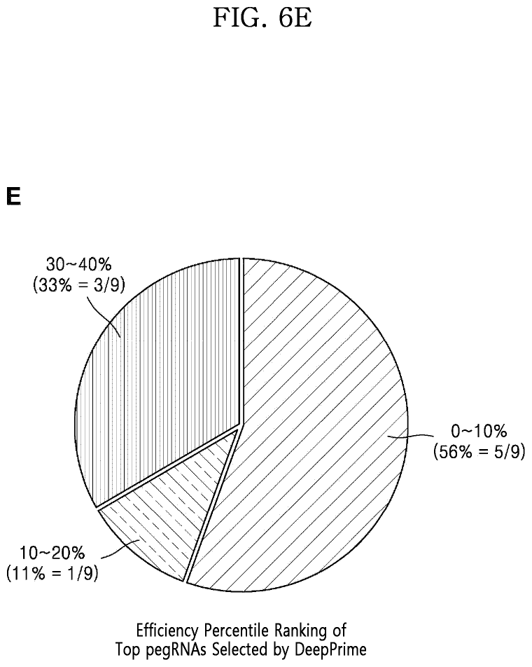

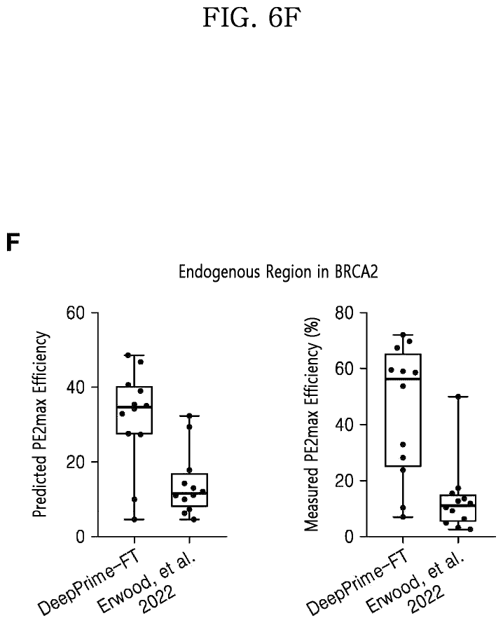

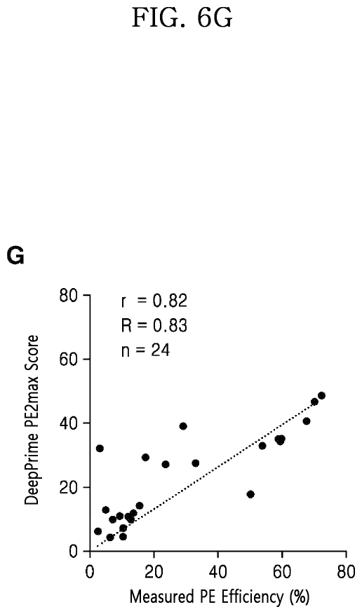

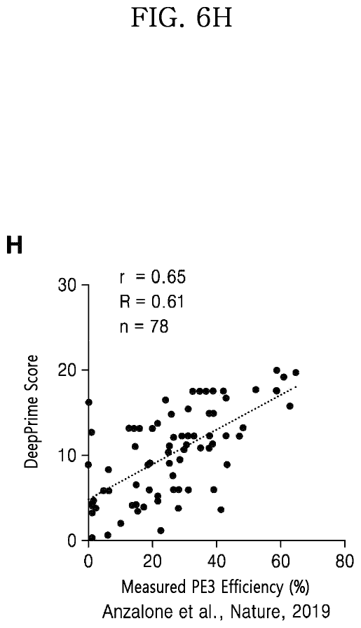

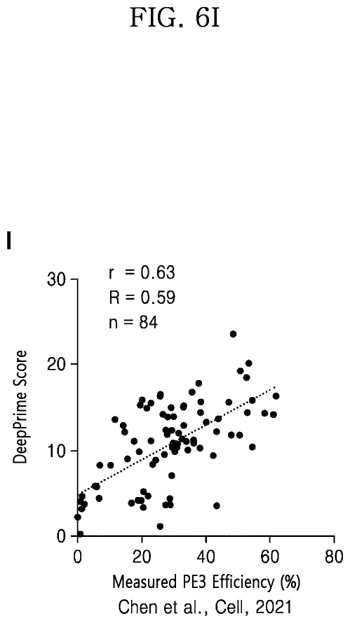

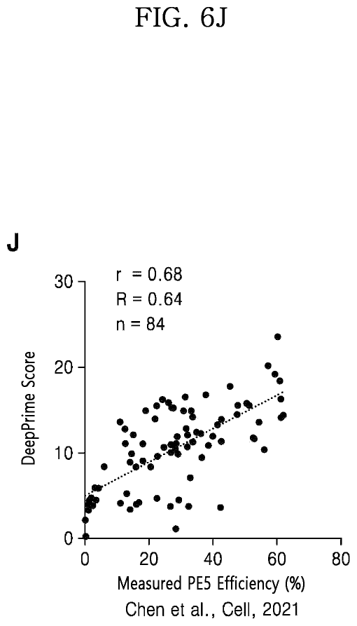

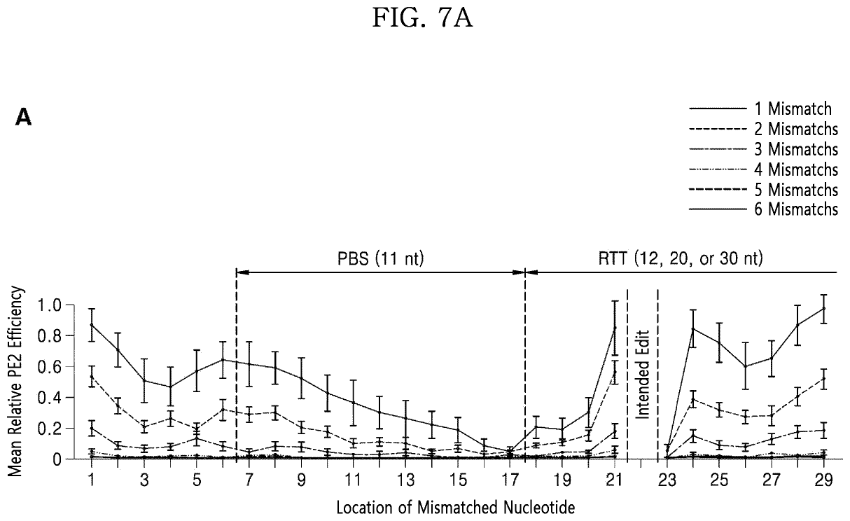

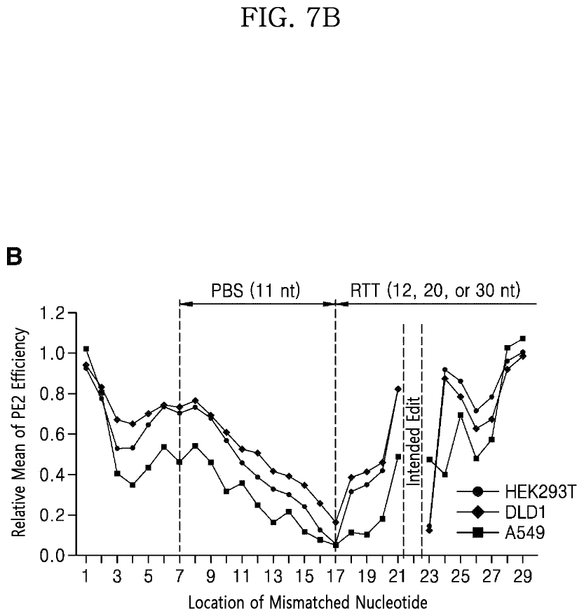

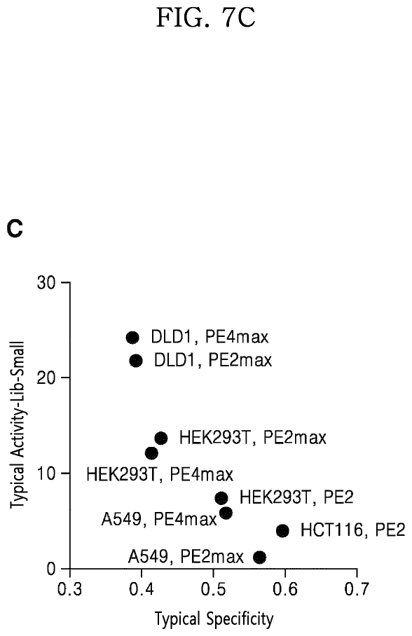

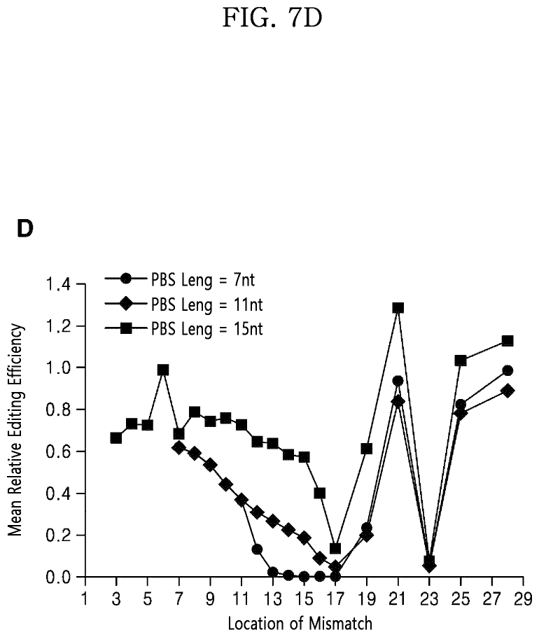

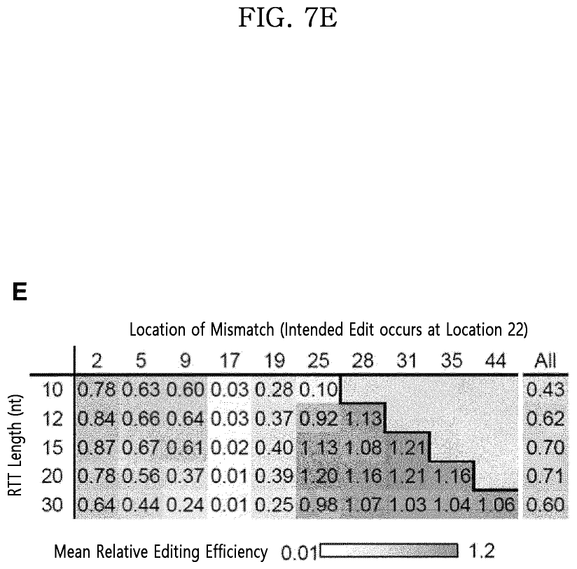

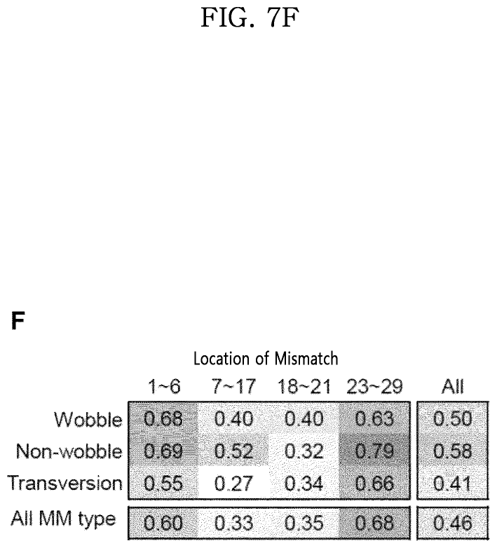

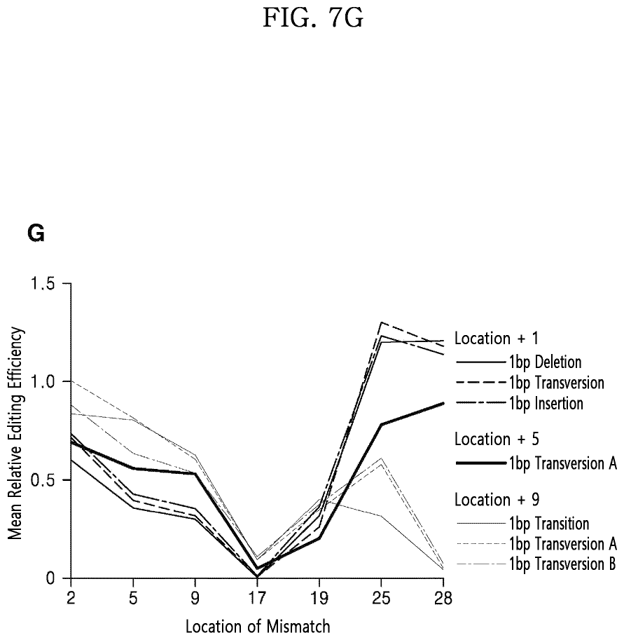

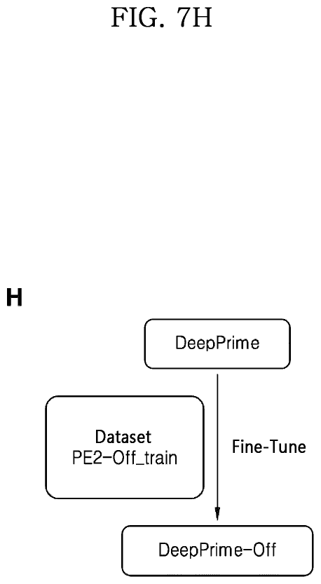

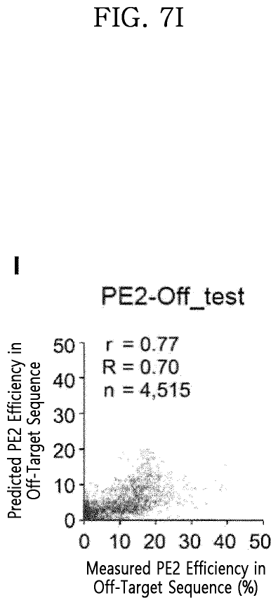

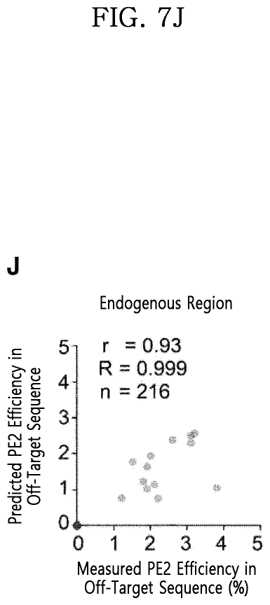

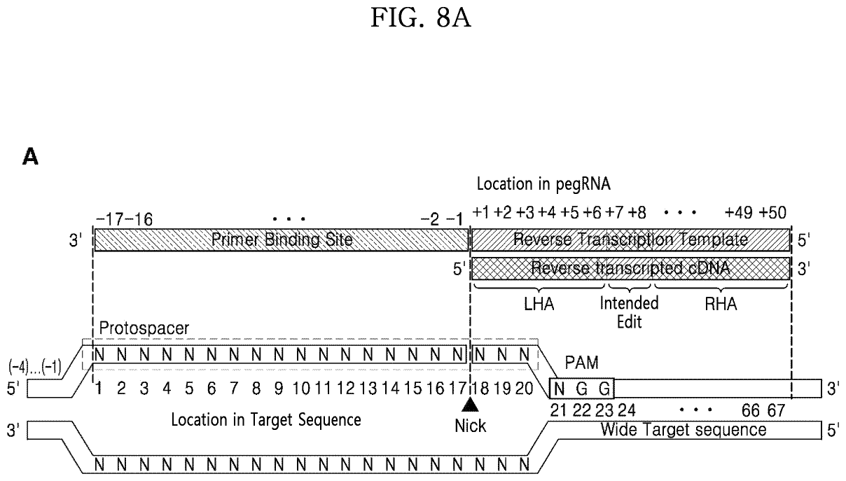

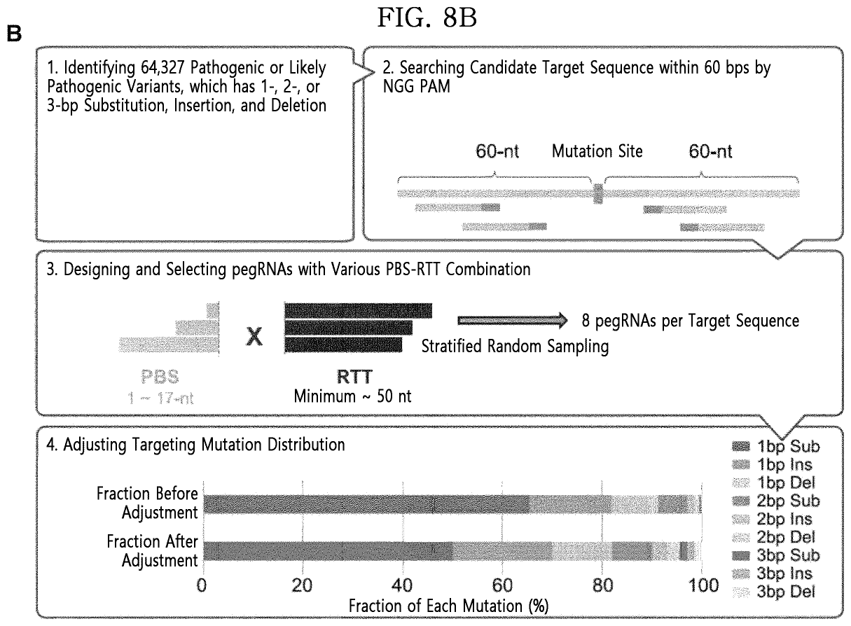

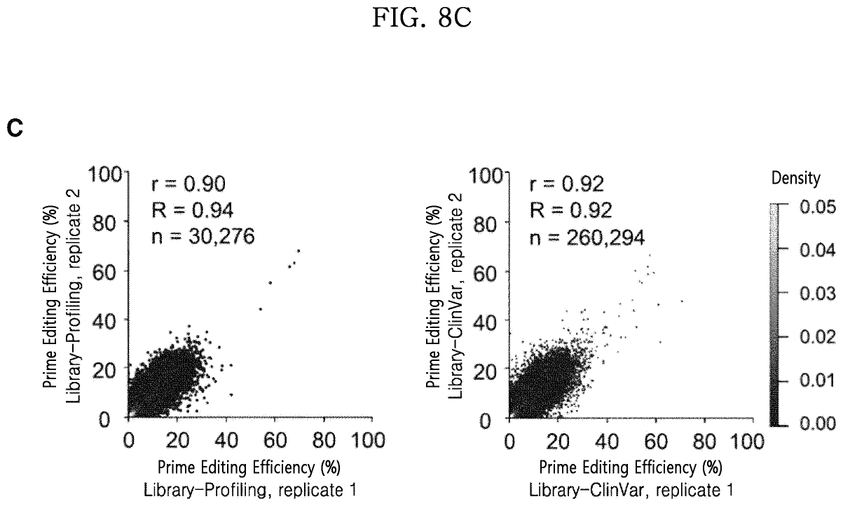

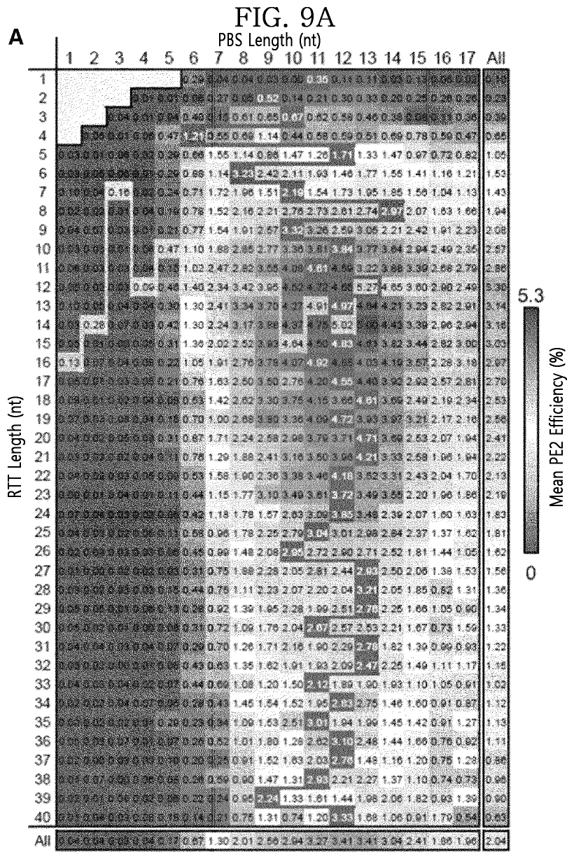

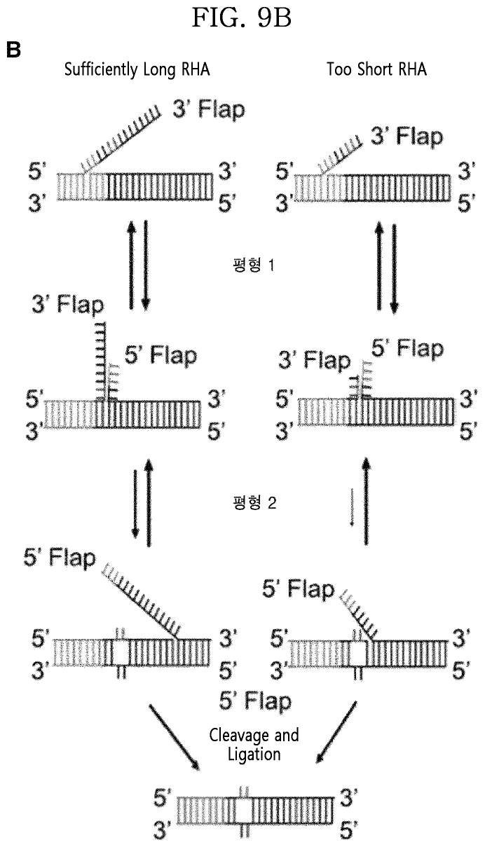

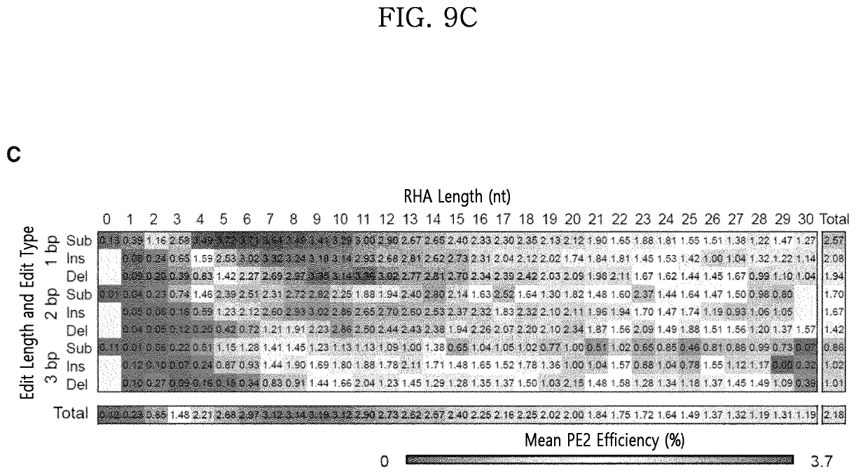

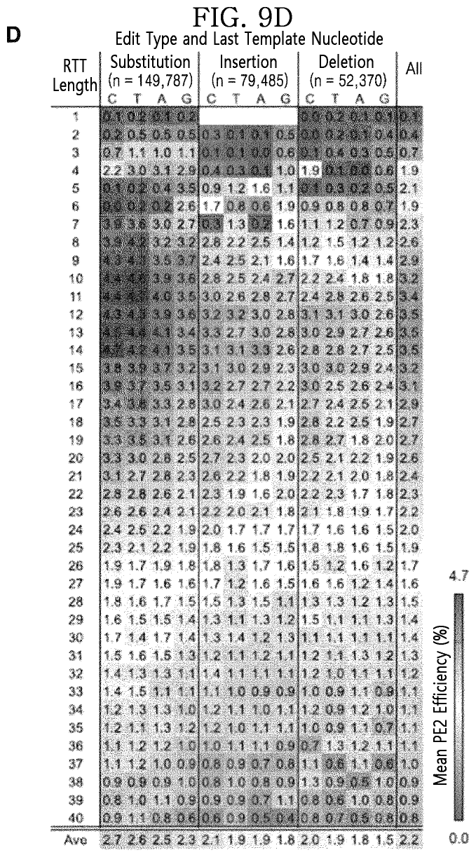

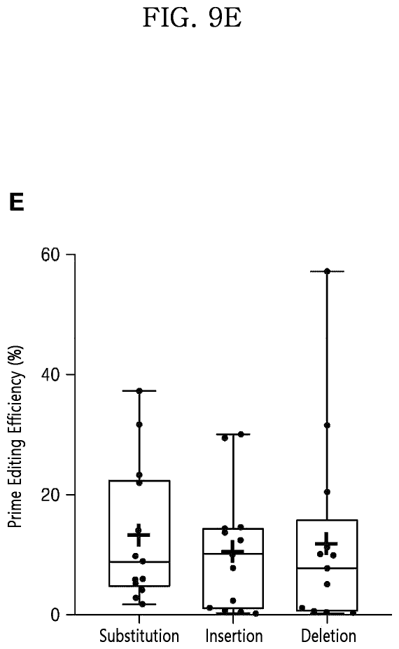

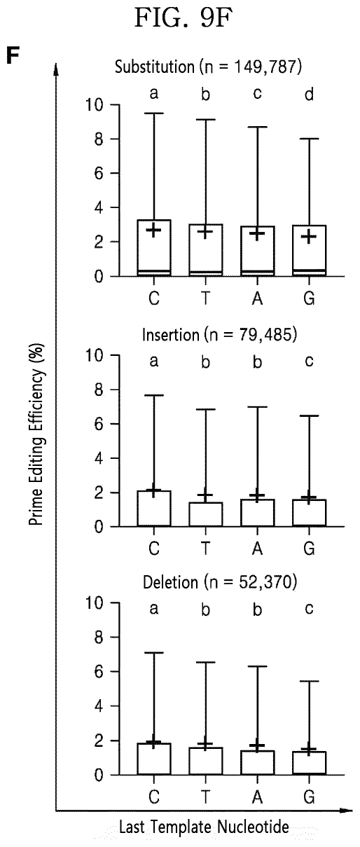

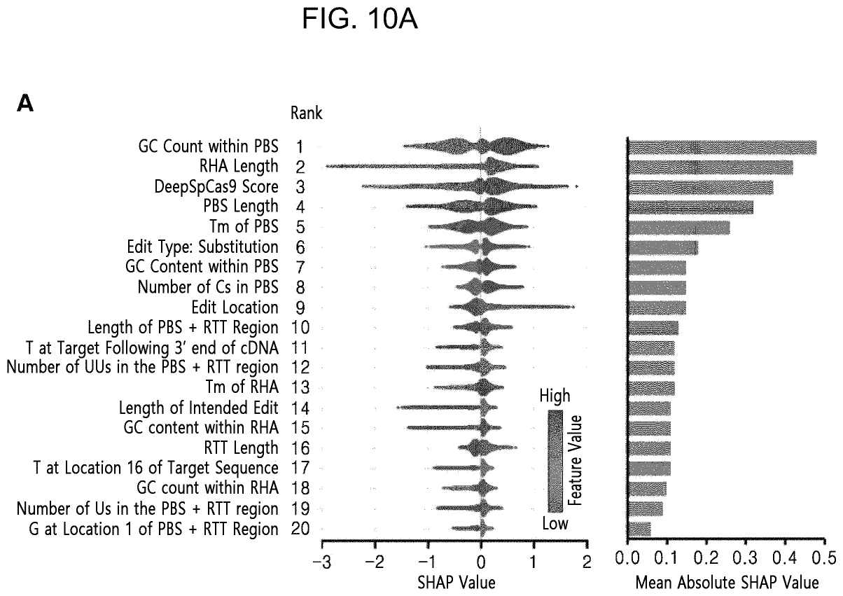

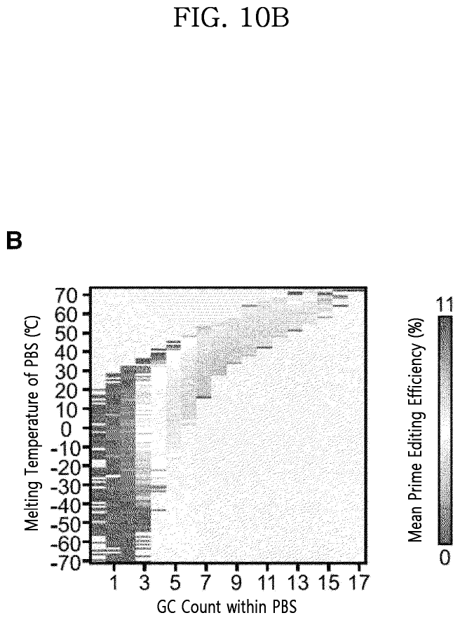

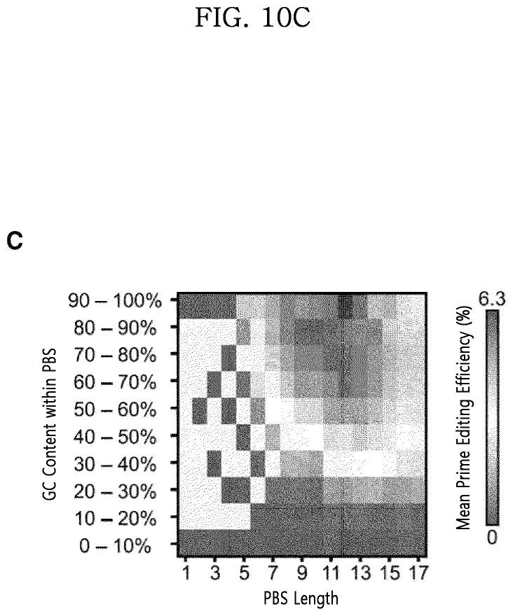

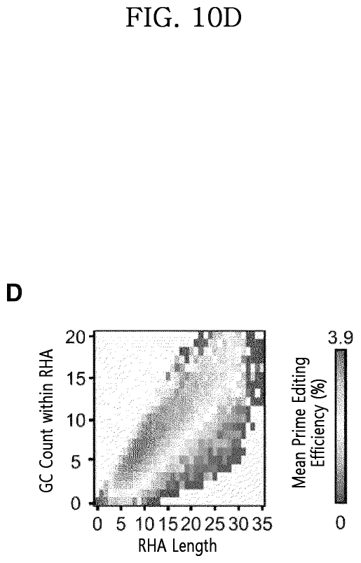

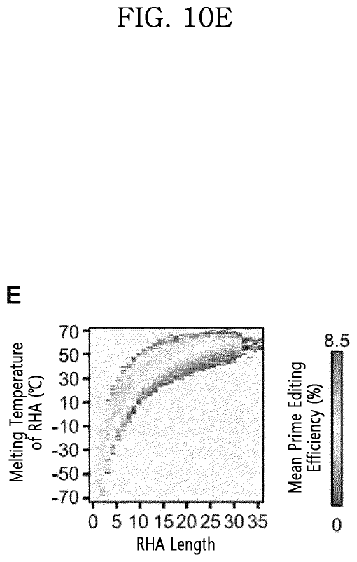

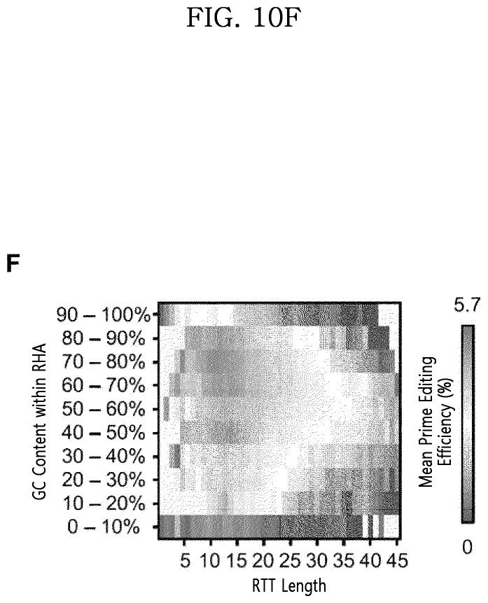

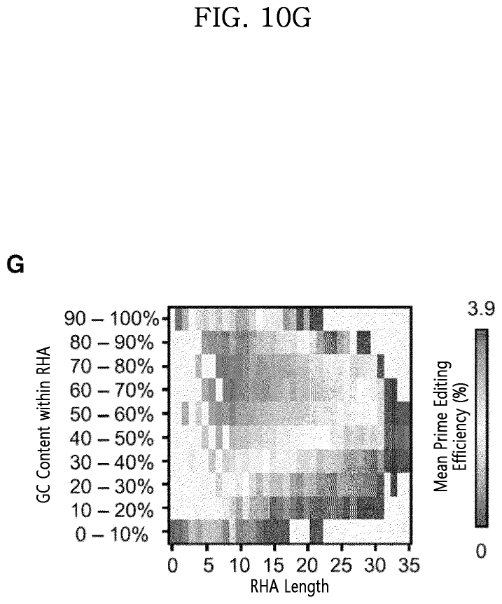

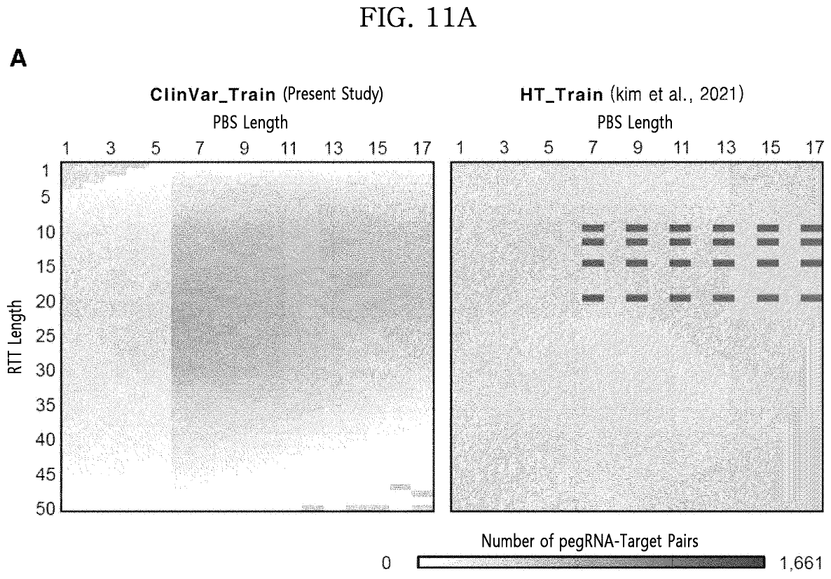

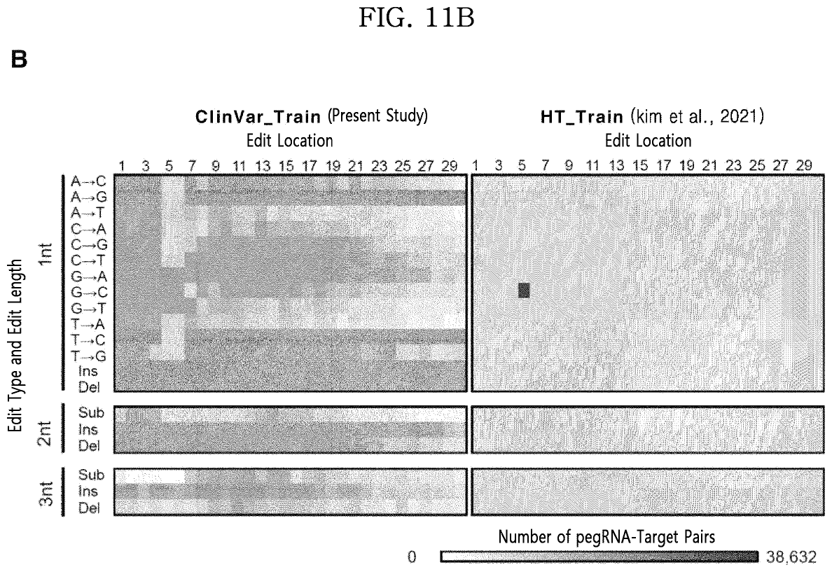

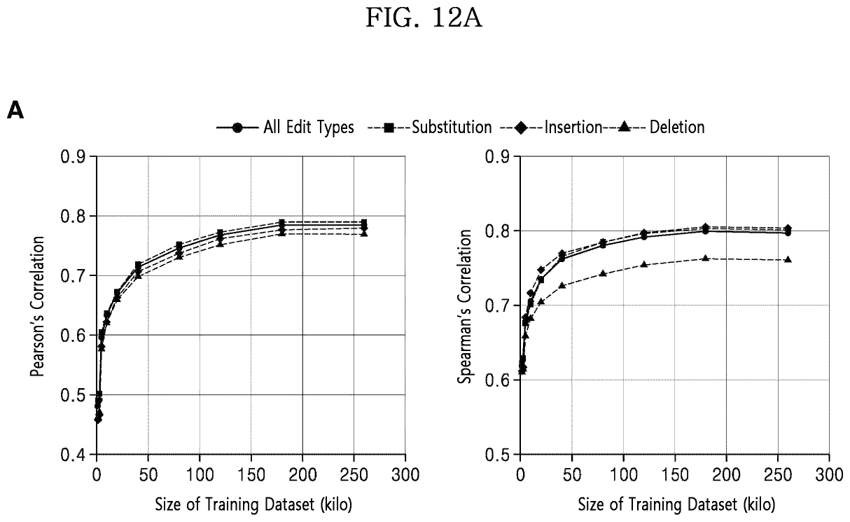

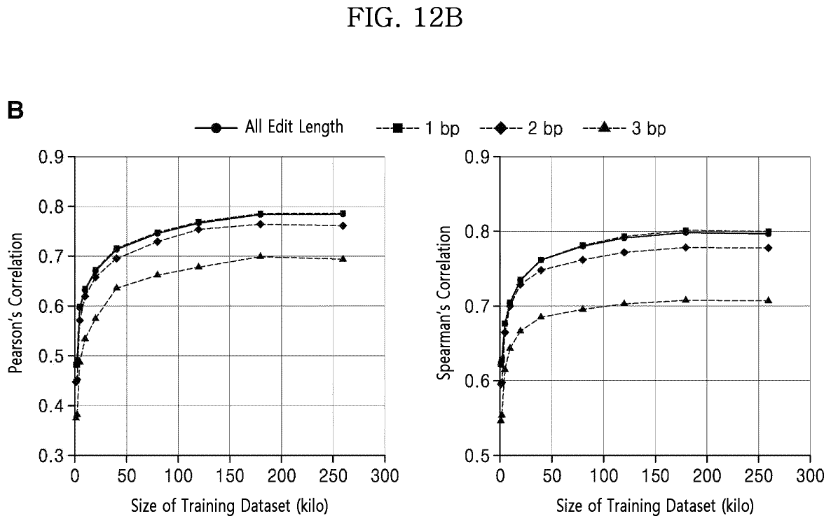

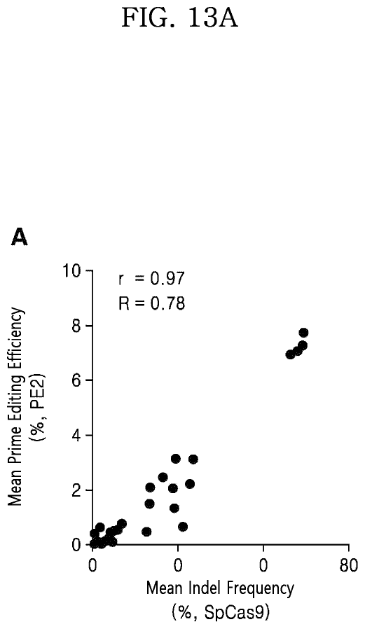

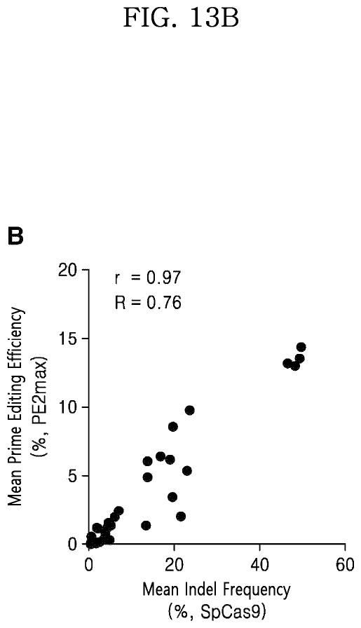

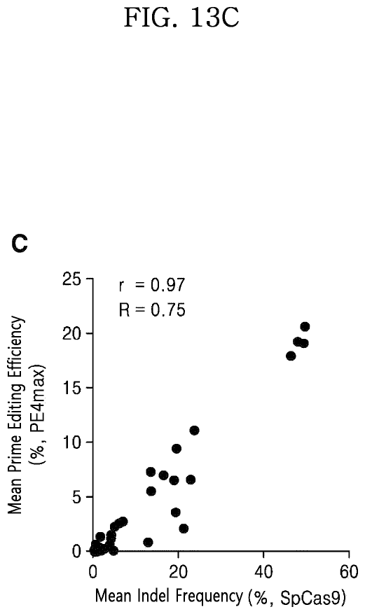

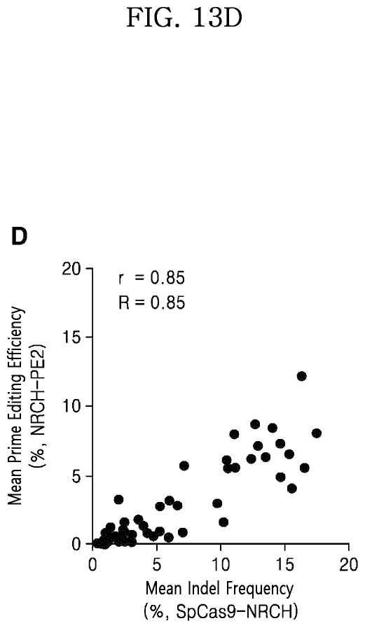

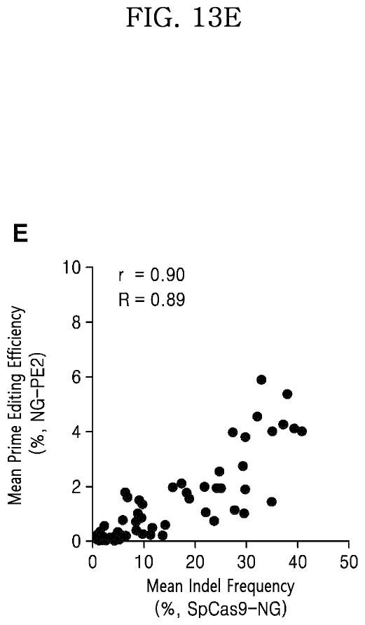

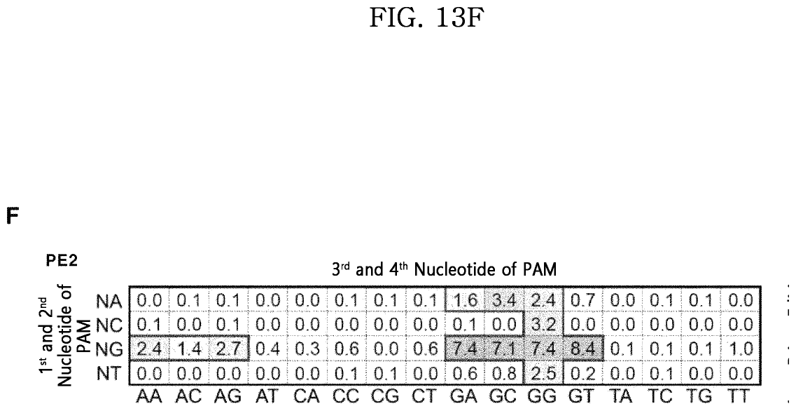

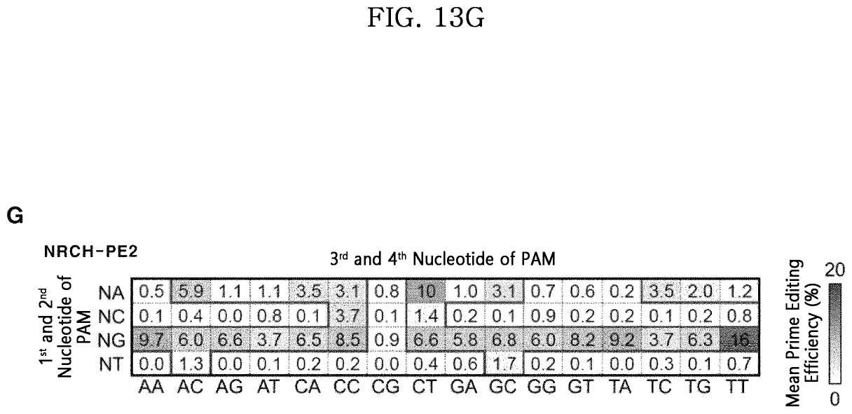

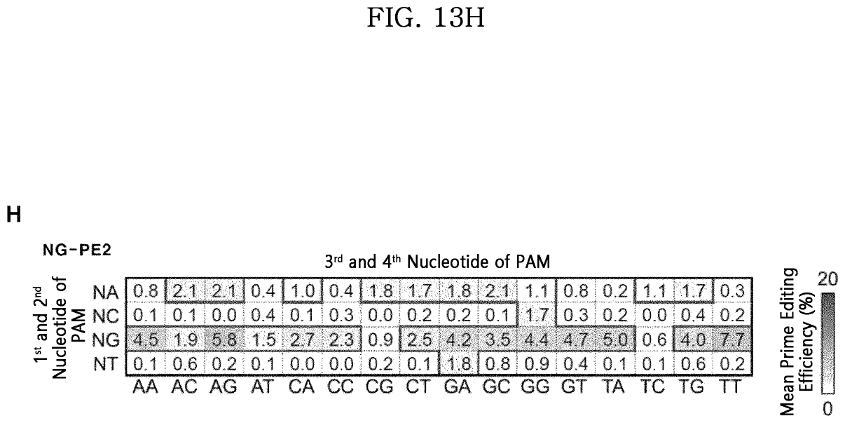

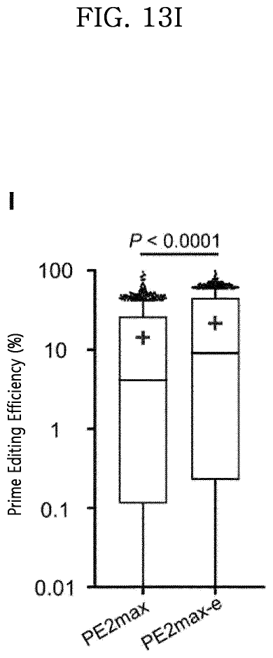

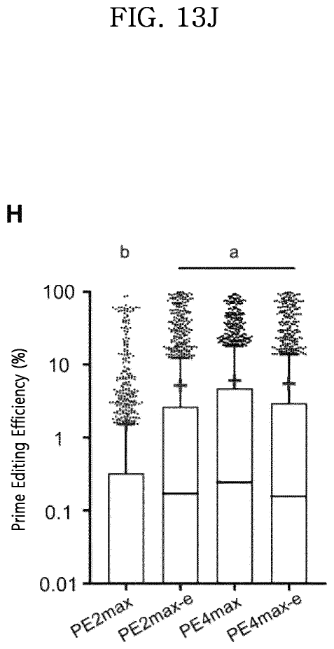

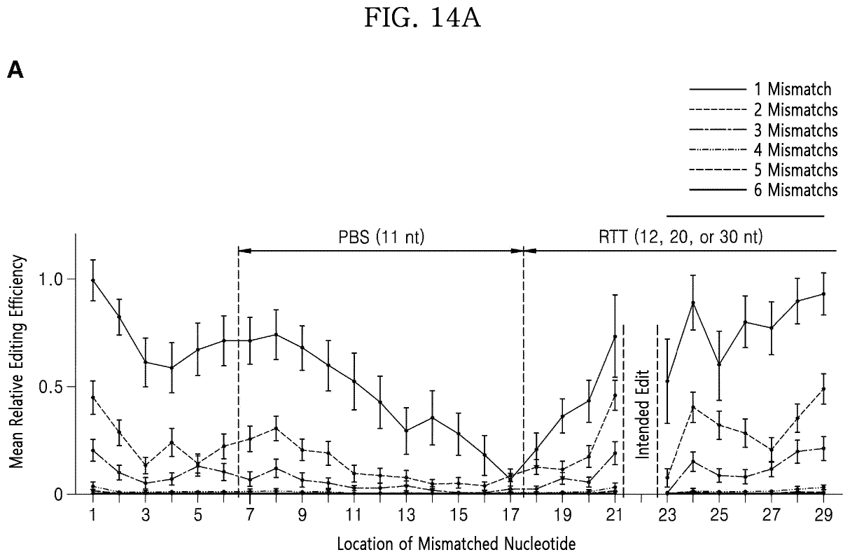

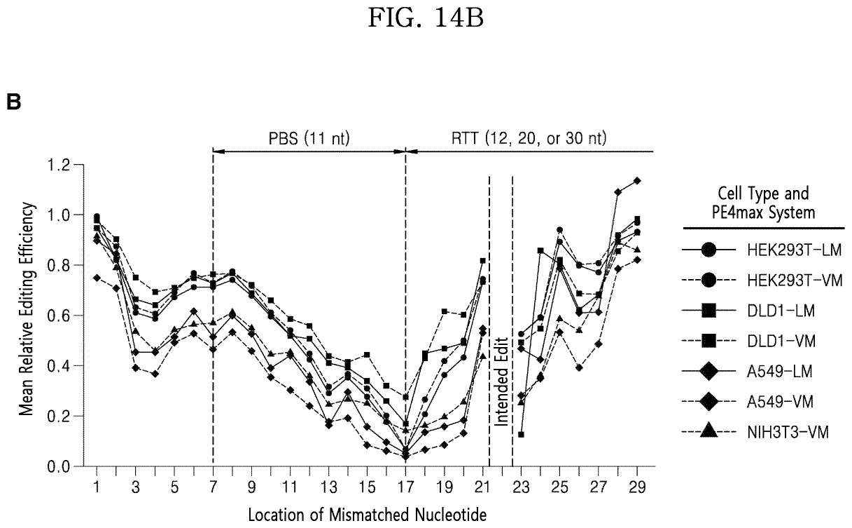

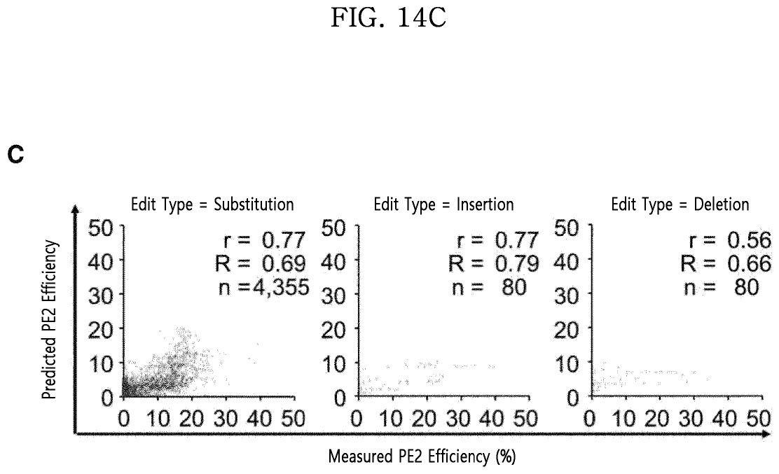

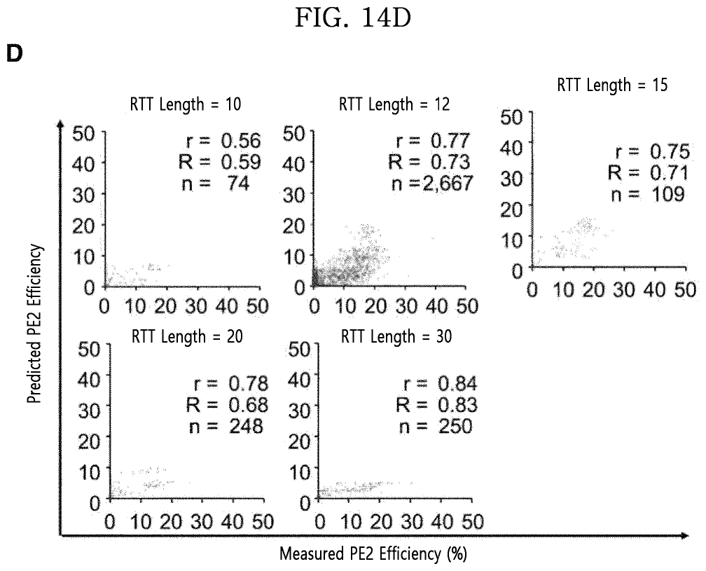

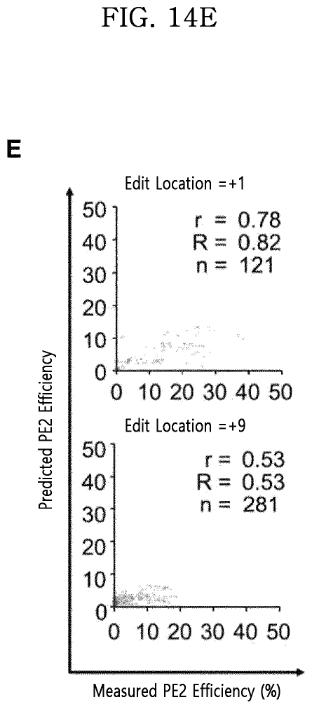

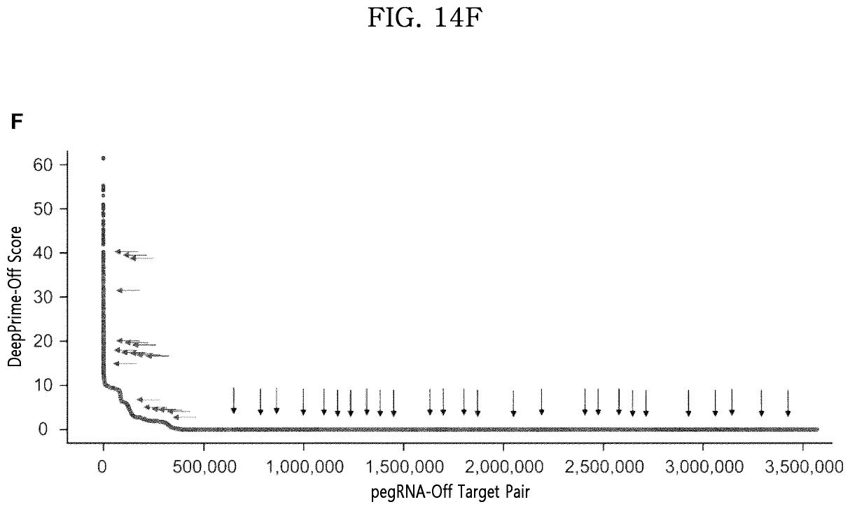

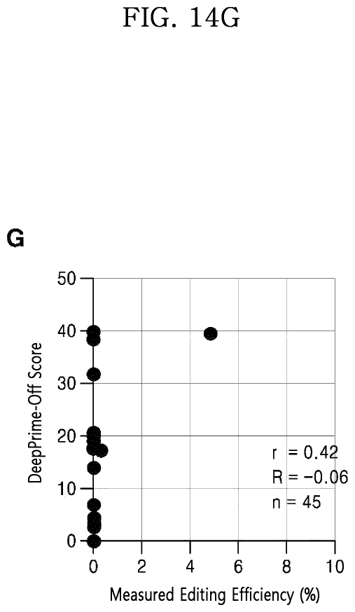

FIG. 8. High-throughput Evaluation of Prime Editing Efficiency Regarding FIGS. 1 and 2

(A) Schematic diagram showing how a pegRNA, CDNA, and wide target sequence are located in this study, herein the locations within the pegRNA and the cDNA generated from the pegRNA are numbered starting from a nicking site of the Cas9 nickase, as for a location within the wide target sequence, the 20th nucleotide upstream from the PAM is location 1, and the nucleotides of the NGG PAM are designated to be at locations 21-23, left homology arm (LHA); right homology arm (RHA); and protospacer adjacent motif (PAM), (B) Schematic diagram showing a selection process of pegRNAs contained in Library-ClinVar, herein pathogenic or likely pathogenic substitutions, insertions, and deletions ranging in size from 1 to 3 bps were selected from the ClinVar database, target sequences were identified, and candidate pegRNAs with randomly determined PBS and RTT lengths (where a minimum length of RTT is determined by locations of edits) were designed, in addition, a proportion of pegRNAs designed to introduce 2-bp and 3-bp variants was increased by reducing a relative number of target sequences per intended 1-bp edit and adding randomly generated 3-bp variants that occurred because most variants in the ClinVar database were single nucleotide variants, and (C) Correlation between prime editing efficiencies obtained from replicates of high-throughput experiments using Library-Profiling (left) and Library-ClinVar (right), herein a color of each dot is determined by density of adjacent dots, and Pearson's (r) and Spearman's (R) correlation coefficients and number (n) of pegRNAs are shown,

FIG. 9. Effects of Lengths of PBS, RTT, and RHA and Last Template Nucleotide on Prime Editing Efficiency regarding FIG. 1

(A) Heat map showing mean prime editing efficiency for lengths of given PBS and RTT, herein yellow and green boxes represent the highest mean prime editing efficiency for each PBS length when tested with all available lengths of the RTT, respectively, and vice versa, (B) Mechanism proposed to explain the effect of RHA lengths on prime editing efficiency, (C) Effect of RHA lengths on prime editing efficiency, herein Heat map showing mean prime editing efficiency for 1-bp to 3-bp substitutions (Sub), insertions (Ins), and deletions (Del) at various RHA lengths, (D) Heat map showing the effect of nucleotides at the last template location on mean prime editing efficiency, herein pegRNAs of Library-ClinVar were organized into groups depending on the RTT lengths and an encoded edit types, and each value in the heat map represents the mean editing efficiency, (E) Effect of edit types on prime editing efficiency in endogenous regions, herein each dot represents efficiency measured for each target sequence, and number of target sequences is (n)=13 per edit type, (F) Prime editing efficiency induced by each of pegRNAs with different nucleotides at the last template location, herein subsets of experimental groups without statistically significant differences (P<0.05, ANOVA followed by Türkiye's post hoc test) in prime editing efficiency are indicated by letters a, b, c, and d in order of mean prime editing efficiency, and (E, F) Presentation of the 25th, 50th, and 75th percentiles by the top, middle, and bottom lines in the box, respectively, and presentation of minimum and maximum values by whiskers, herein plus sign in the box plot indicates the mean prime editing efficiency,

FIG. 10. PBS and RHA Features Associated with Prime Editing Efficiency regarding FIG. 2

(A) The 20 most important features associated with prime editing efficiency determined by Tree SHapley Additive explanations (SHAP), herein in summary, the violin plots (left graph) show each pair of pegRNAs and target sequences as a dot such that dot positions on the x-axis reflects SHAP values, a high SHAP value indicates that its corresponding feature is associated with high prime editing efficiency, red or blue dots indicate high or low values of the relevant features, respectively, overlapping dots were slightly spaced apart in the y-axis direction to make density clearly visible, Tm represents melting temperature, and number of pegRNA and target sequence pairs (i.e., number of points per feature in summary plot) is n=259, 910, and (B-G) Effect of PBS-associated features (B, C) and RHA-associated features (D-G) on mean prime editing efficiency, herein (B-G) A total of 288, 793 pegRNAs included in Library-ClinVar were used for analysis,

FIG. 11. Comparison of Training Datasets for DeepPrime and DeepPE regarding FIG. 3

(A) Range comparison of PBS-RTT combinations in ClinVar_Train and HT_Train, herein heat map shows the number of pairs of pegRNAs and target sequences for given PBS-RTT combinations, and PBS-RTT combinations excluded from analysis are shown in gray, and (B) Comparison of the range of edit types, lengths, and positions between ClinVar_Train and HT_Train, herein heat map represents the number of pairs of the pegRNAs and target sequences for given intended edits, and intended edits excluded from the analysis are shown in gray,

FIG. 12. Performance of Deep Learning-based Computation Models Predicting Prime Efficiency of PE2 depending on Size of Training Dataset regarding FIG. 3

Small-sized training datasets were created by randomly subsampling the training dataset for DeepPrime, performance of models depending on edit types (A) and edit lengths (B) is shown, and values displayed on the Y axis represent the mean of 5-fold cross-validation, and

FIG. 13. The impact of PAM compatibility of PE2, NRCH-PE2, and NG-PE2, and epegRNA on PE efficiency, regarding FIG. 4

(A-E) Correlation between the activity of SpCas9 nuclease and PE2 variants in target sequences with the same PAM sequence. Mean indel frequency induced by SpCas9 variants and mean prime editing efficiency induced by PE2 variants in target sequences s with 64 different PAM sequences (NXXX, where X varies). Each dot represents the result for each NXXX PAM sequence. Pearson's (r) and Spearman's (R) correlation coefficients are shown. Number of analyzed PAM sequences n=64. (F-G) Heatmap showing the mean prime editing efficiency induced by PE2 (F), NRCH-PE2 (G), and NG-PE2 (H) in target sequences including 64 different PAM sequences. Among these 64 possible PAM sequences, sequences with an average prime editing frequency higher than 1% are outlined in red (bold). (I, J) Comparison of prime editing efficiency using pegRNA and epegRNA in HEK293T and A549 cell lines. Prime editing using epegRNA is indicated by adding “-e” to the name of the prime editor (e.g., PE2max-e). Number of tested target and pegRNA pairs (n)=1,469 (I, HEK293T cell line) and (n)=2,001 (J, A549 cell line). The upper, middle, and lower lines in the box represent the 25th, 50th, and 75th percentiles, respectively, and the whiskers represent the minimum and maximum values. The plus sign in the box plot represents the average prime editing efficiency. (I) Statistical significance (paired t-test) is indicated. (J) Subsets of experimental groups with no statistically significant differences (P<0.05, ANOVA followed by Tukey's post hoc test) in prime editing efficiency are indicated by letters a and b in order of average prime editing efficiency.

FIG. 14. High-throughput Evaluation and Prediction of Prime Editing Efficiency on Off-target Sequences regarding FIG. 7

(A) Effect of number of the mismatches on prime editing efficiency induced by a PE4max system on off-target sequences in HEK293T cell line, (B) Effect of locations of the mismatches on prime editing efficiency induced by the PE4max system on off-target sequences in HEK293T, DLD1, A549, and NIH3T3 cell lines, herein LM, lentiviral transmission of a dominant negative form of MLH1 (MLH1dn); VM, delivery of MLH1dn using engineered virus-like particles, (A, B) relative editing efficiency was calculated by dividing the prime editing efficiency in the off-target sequences by the on-target efficiency in the on-target sequences, the pegRNAs used in this analysis included PBS with a length of 11-nt and RTT with a length of 12-nt, 20-nt, or 30-nt, the intended edit was a G to C transversion encoded at location +5 of the pegRNAs, all possible 1-bp mismatches at locations 1 to 29 of the target sequences were included in the 1-bp mismatch population, the type and location of the mismatches were randomly selected in the 2-bp to 6-bp mismatch population (see methods for details), error bars represent 95% confidence intervals, (C-E) Evaluation of DeepPrime-Off, herein PE-Off_Test dataset was split into 3, 5, and 2 data subsets depending on intended edit types (C), RTT length (D), and locations of the edits (E), respectively, and these data subsets were used to evaluate the model, (F, G) Application of DeepPrime-Off to identify non-target regions, distribution of DeepPrime-Off scores for 3,571,521 pairs of pegRNAs and potential off-target regions with up to 3 mismatches, herein black arrows (along the x-axis) and purple arrows (along the y-axis) indicate pairs selected for experimental evaluation as follows, the black and purple arrows are 26 pegRNAs with no predicted off-target effects in off-target regions (along the x-axis) and 19 pegRNAs with predicted off-target effects in the off-target regions (along the y-axis), respectively, (G) Experimental evaluation of 45 (=26+19) pairs selected in (F), and (C-E, G) Pearson's (r) and Spearman's (R) correlation coefficients and number of pegRNA and target sequence pairs (n) are shown.

### BEST MODE

Hereinafter, the present disclosure will be described in more detail through embodiments. However, these embodiments are for illustrative purposes only and the scope of the present disclosure is not limited to these embodiments.

### Methods

### Data and Code Availability

Targeted deep sequencing data in this study was submitted as PRJNA906920 into NCBI Sequence Read Archive (SRA; https://www.ncbi.nlm.nih.gov/sra/). Source codes for DeepPrime, DeepPrime-FT, and DeepPrime-Off, and custom Python script to calculate prime editing efficiency are available at https://github.com/yumin-c/DeepPrime and https://github.com/hkimlab/DeepPrime.

### Cell Line Culture and Selection Conditions

HEK293T, HCT116, HeLa, DLD1, A549, and NIH3T3 cells were cultured in Dulbecco's Modified Eagle Medium (DMEM, Thermo Fisher Scientific) supplemented with 10% fetal bovine serum (FBS, RDT). An MDA-MB-231 cell line was cultured in RPMI 1640 medium containing HEPES (Thermo Fisher Scientific) supplemented with 10% FBS. All cell lines were maintained below 80% confluency at 37° C. and under 5% CO₂ condition, and the cell lines were sub-cultured every 3-4 days. Some of the cells transduced with PE2-encoding or PE variant-encoding lentivirus were selected using 10 pg/mL blasticidin S (BSD). Others of the cells with pairwise libraries were selected using 1 pg/mL puromycin.

### Oligonucleotide Library Design

Oligonucleotide pools containing pegRNA-target sequence pairs were synthesized by Twist Bioscience (San Francisco, CA). Each oligonucleotide contained the following elements. These included 19-nt guide sequences, BsmBI restriction sites #1, 10-nt to 15-nt barcode sequences (barcode 1), BsmBI restriction sites #2, RTT sequences, PBS sequences, poly-T sequences, 14-nt to 18-nt barcode sequences (barcode 2), and 74-nt wide target sequences containing PAM and RTT binding sites (FIG. 1A). The barcode 1 was included to minimize template switching during PCR amplification. The barcode 2 (located upstream of the target sequences) was included to identify individual pairs of the pegRNAs and target sequences after deep sequencing. Oligonucleotides containing unintended BsmBI restriction sites in their sequences were excluded.

### Library-Profiling Design

To evaluate factors affecting prime editing efficiency, the present inventors designed a library of 47, 839 pairs of pegRNAs and target sequences, and the present inventors named it Library-Profiling. The present inventors selected 40 seed target sequences (i.e., 20 nucleotides proximal to the PAM) that showed a high level of SpCas9-induced indel frequency in a previous study (Kim et al., 2019). In 20 of those 40 seed target sequences, paired sgRNAs induced an indel frequency of 70%-75%. In the other 20, paired sgRNAs induced an indel frequency of 50%-55%. For each of these seed target sequences, pairs of 74-nt wide target sequences (FIG. 1A) and pegRNA sequences were prepared, the 4-nt wide target sequences and pegRNA sequences having a wide range of PBS and RTT lengths and a variety of PBS and RTT locations, edit lengths, and edit types. A total of 81 oligonucleotides containing the BsmBI cleavage sites were excluded. As detailed in the list below, pegRNA-target sequence pairs were classified into seven groups. Within Library-Profiling, specific pegRNA-target pairs were evaluated but excluded from the final analysis.

i. Effect of PBS lengths: The pegRNAs contained RTTs with a fixed length of 5, 12, 20, 33, and 50 nts, respectively, each of which was combined with PBSs with a length of 1 to 17 nts. An intended edit was set to be a +5 G to C transversion.

ii. Effect of RTT lengths: The pegRNAs contained PBSs with a fixed length of 7, 12, and 17 nts, respectively, each of which was combined with RTTs with a length of 5 to 40 nts as well as with RTTs with a length of 42, 44, 46, 48, and 50 nts. An intended edit was set to be +5 G to C transversion.

iii. Effects of edit locations: The pegRNAs contained PBSs with a length of 12-nt, each of which was combined with RTTs with a length of 5, 12, 20, 30, and 50 nt, respectively. The intended edits included all possible 1-bp substitutions (A*C*G*T to T*G*C*A) at all edit locations.

iv. Effects of edit types: The pegRNAs contained PBSs with a length of 12-nt, each of which was combined with RTTs with a length of 1 to 40 nt. The intended edits were 1-bp substitutions, 3-bp insertions (AGG or CCT), or 3-bp deletions at locations +1, +5, +12, and +20 from the nicking site. The minimum RHA length was 0 nt for substitutions and insertions and 1 nt for deletions.

v. Effects of PAM co-editing: The pegRNAs contained PBSs with a length of 12-nt, each of which was combined with RTTs with a length of 22-nt. 1-bp substitutions from A*C*G*T to T*G*C*A were designed at locations +1, +2, +3, +4, and +8 from the nicking site. At the same time, it was designed to co-edit all 16 possible PAMs simultaneously at locations +5 and +6 (i.e., in NGG PAMs).

vi. Effect of substituted nucleotide number: The pegRNAs contained PBSs with a length of 12-nt, each of which was combined with RTTs with a length of 22-nt. Among locations +1, +2, +4, +7, +8, +9, +10, +11, +12, +13, and +14 from the nicking site, edit locations of up to +10 were randomly selected for replacement installation. The random selection of 1 to 10 edit locations was repeated five times, resulting in 55 pegRNAs per seed target sequence.

vii. Effect of inserted or deleted nucleotide number: The pegRNAs contained PBSs with a length of 12-nt, each of which was combined with RTTs with a length of 22-nt. The intended edits were 1-nt to 10-nt, 12-nt, 15-nt, or 20-nt insertions or 1-nt to 10-nt, 12-nt, 15-nt, 20-nt, or 30-nt deletions. The edits were designed to be installed at locations +2, +5, +10, and +15 from the nicking site. Insertions were derived from two template sequences: type I: ‘AGGATCGATCCTGTACTTGC’ and type II: ‘CCTGACAACGCTTAGACAGA’. Insertions of the desired size were spliced at the 5′ end of the template sequences. For example, an intended 4-bp insertion would generate two pegRNAs to insert AGGA or CCTG for type I and type II, respectively.

### Library-ClinVar Design

To evaluate prime editing efficiency for installation and correction of disease-related mutations, the present inventors designed a library of 549,168 pairs of pegRNAs and target sequences. The present inventors selected 64, 326 unique variants from the ClinVar database (Version Apr. 20, 2020) (Landrum et al., 2016) of 1-bp to 3-bp contiguous substitutions, insertions, or deletions and also classified as pathogenic or likely pathogenic. The present inventors then extracted all possible guides containing NGG PAM sequences within a 60-nt flanking window where ClinVar variants could be introduced or modified. 74-nt target sequences containing each variant were extracted. An edit window ranged from +1 to +30 for all possible combinations of PBS lengths (1 to 17 nt) and RTT lengths (a range from the minimum length required to edit a variant to a maximum of 50 nt). As a result, pegRNAs were designed. A final pegRNA library was made from eight randomly selected pegRNAs for each seed target. Most of the variants from the ClinVar database were single nucleotide variants. Due to that, the relative number of target sequences requiring 1-bp editing was reduced, thereby the proportion of the pegRNAs for introducing 2-bp and 3-bp variants was increased. For 3-bp insertions and substitutions, the number of target sequences for these variant types was limited. Due to that, randomly generated variants were included. Finally, oligonucleotides containing internal BsmBI cleavage sites were removed during the selection process.

### Library-Small Design

To evaluate the editing efficiency of PE variants and pegRNAs using conventional or optimized scaffolds in various cell lines, the present inventors prepared Library-Small of 6,000 pairs of pegRNAs and target sequences. First, 2,990 pegRNA-target sequence pairs (1, 495 pairs for disease modeling and 1,495 pairs for therapeutics) were selected from the ClinVar_Train dataset. Half of the pairs were randomly selected, and the remaining half were proportionally selected from the editing efficiency ranges of 0%, 0%-1%, 1%-5%, and 5% or higher. Additionally, to determine the PAM compatibility of PE variants, 2,990 additional pairs of pegRNAs and target sequences were prepared by randomly changing the NGG PAM sequences to NNN sequences. Finally, 20 pegRNAs that showed the highest editing efficiency in a previous study were included as positive controls with 5-fold overlap (5×4 pegRNAs).

### Library-epegRNA Design

To evaluate the editing efficiency of engineered pegRNAs (epegRNAs), a Library-epegRNA was prepared from 6,000 epegRNA sequences (each with an 8-nt linker and tevopreQ1 structural motif at the 3′ end) paired with the corresponding target sequences. Except for added linkers, these 6,000 epegRNA sequences were identical to those of Library-Small. An 8-nt linker sequence for each epegRNA was designed using a pegRNA linker identification tool (pegLIT).

### Library-Off Design

To evaluate factors affecting the specificity of prime editing, the present inventors prepared Library-Off of pegRNA sequences paired with perfectly matched on-target and off-target sequences, respectively. The pegRNAs were selected because the pegRNAs showed high prime-editing efficiency in the previous and current high-throughput studies. To systematically examine factors affecting prime efficiency specificity, the components of Library-Off were organized into six groups. Each group was made from 40 on-target seed sequences with specific mismatch patterns or with pegRNA features to deal with different aspects of prime editing specificity. These target sequences contained 4 common seed sequences that showed high efficiency in a previous study and 31 in a current study. In addition, 5 common seed sequences were contained. However, when there are two or more mismatches in the target sequences, 10 additional seed sequences were included to examine prime editing specificity more closely. The Library-Off was made from a total of 48,263 pegRNA-target pairs excluding oligonucleotides used in other analyses or oligonucleotides containing internal BsmBI cleavage sites. Each non-targeted profiling group was described in detail below.

i. Effect of mismatch locations on single base resolution: Target sequences were designed to examine an effect of 1-bp mismatches at all target sequence locations (1-29) corresponding to all regions interacting with pegRNA. The pegRNAs were designed as PBSs with a length of 11-nt and RTTs with a length of 12-nt. The intended edit was set to be a +5 G to C transversion.

ii. Effect of RTT lengths: The pegRNAs contained PBSs with a length of 11-nt, each of which was combined with RTTs of lengths 10, 12, 15, 20, and 30 nt, respectively. Mismatches in the target sequences were distributed at locations 2, 5, 9, 17, 19, 25, 28, 31, 35, and 44. An intended edit was set to be +5 G to C transversion.

iii. Effect of PBS lengths: The pegRNAs contained RTTs with a length of 12-nt, each of which was combined with PBSs with lengths of 7, 11, and 15 nt, respectively. Mismatches in the target sequences were distributed in regions corresponding to each of the PBSs and at locations 19, 21, 23, 25, and 28. An intended edit was set to be +5 G to C transversion.

iv. Effect of type of edits on off-target prime editing: The pegRNAs contained PBSs with a length of 11-nt, each of which was combined with RTTs with a length of 12-nt. The intended edits were set to induce a +5 G to C transversion with a 1-bp transition (GC*AT), a 1-bp deletion, and a G insertion. Alternatively, the intended edits were set to induce two types of 1-bp transitions (GC*AT and GT*AC) and a 1-bp transversion (AG*CT) at locations +1 or +9 from a nicking site, respectively. Mismatches in the target sequences were distributed at locations 2, 5, 9, 17, 18, 25, and 28.

v. Effect of mismatch number: To evaluate prime editing under these conditions, targets with 2 to 6 mismatches to their corresponding pegRNAs were designed. The pegRNAs contained PBSs with a length of 11-nt, each of which was combined with RTTs with lengths of 12, 20, and 30 nt, respectively. The type of an intended edit was set to be a +5 G to C transversion. From target locations 1 to 29 (all locations corresponding to their pegRNA binding sites when the pegRNAs contains 12 nt long RTTs), 2 to 6 locations were randomly selected to introduce mismatches. For pegRNAs containing RTTs with a length of 12-nt, 100 seed targets with two mismatch locations were selected, and 50 seed targets were selected for the remaining mismatch number (3 to 6). For pegRNAs containing RTTs with a length of 20-nt or 30-nt, 10 seed targets were selected for each tested mismatch number (2-6).

vi. Off-target control: Previously reported (Kim et al., 2020a) pairs of pegRNAs and their corresponding off-targets were included as a control.

### Plasmid Library Construction

Plasmid libraries containing pairs of pegRNA-encoding sequences and their corresponding target sequences were prepared using the following two-step cloning process. (Step I) Gibson assembly and (Step II) restriction enzyme-directed cleavage and ligation. Using a two-step cloning process adapted and modified from a previously reported method (Shen et al., 2017), separation between the paired guide sequences and target sequences was prevented during oligonucleotide amplification through PCR (Du et al., 2017).

Step I: Construction of initial plasmid libraries containing pairs of pegRNA-coding sequences and target sequences. In each case, an oligonucleotide pool was amplified through 15 cycles of PCR using Phusion Polymerase (NEB), and the oligonucleotide pool was gel purified. Lenti_gRNA-Puro plasmids (Addgene #84752) and Lenti_gRNA-Puro-hMLHdn plasmids were digested with a BsmBI enzyme (NEB) at 55° C. for at least 3 hours. Next, the linearized vectors were treated with 1 μL of Quick CIP (NEB, M0525L) at 37° C. for 10 minutes, and then gel purification was performed. Using the Gibson assembly, the amplified oligonucleotide pool was assembled with linearized Lenti_gRNA-Puro or Lenti_gRNA-Puro-hMLHdn vectors. The assembled products were concentrated using isopropanol precipitation and then transformed into electrocompetent cells (Lucigen) using a MicroPulser (BioRad). SOC medium was then added to the transformation mixture, which was incubated at 37° C. for 1 hour. The cells were then plated and incubated on Luria-Bertani (LB) plates agar containing 50 pg/mL carbenicillin. Small aliquots (0.1, 0.01, and 0.001 μL) of the culture were plated separately to determine library coverage. Plasmids were extracted from whole harvested colonies using the QIAGEN Plasmid Maxi kit (QIAGEN). The calculated coverage of these initial plasmid libraries was 986×, 2,486×, 2,210×, and 500× the number of the oligonucleotides in each library, respectively for the Library-Profiling/ClinVar, Library-Off, Library-Small-PE2, and Library-Small-PE4.

Step II: sgRNA scaffold insertion. In each case, the initial plasmid libraries generated in the Step I were digested with the BsmBI enzyme for at least 6 hours and then treated with 1 μL of Quick CIP for 10 minutes at 37° C. The size of the digested products was selected on a 0.6% agarose gel, and then the digested products were gel purified. The insert DNA fragment containing the existing or optimized scaffold sequences, the DNA fragment being obtained from lentiGuide-Puro plasmids (Addgene #52963) or chemically synthesized oligonucleotides (IDT), respectively, were then PCR amplified using Phusion DNAB polymerase and a primer set containing the BsmBI recognition sequences. Then, T-blunt vector cloning (Solgent) was performed. T-blunt vectors were digested with the BsmBI enzyme for at least 12 hours and gel purified on 2% agarose gel to isolate existing or optimized scaffold sequences with appropriate 5′- and 3′-overhangs. The purified inserts were ligated with the digested initial plasmid library vectors (vector:insert=1:10, w/w) using T4 ligase (Enzynomics) for 3 hours at 16° C. The ligation products were purified using isopropanol precipitation and electroporated into Endura electrocompetent cells (Lucigen). Colonies were harvested, and the final plasmid library was extracted using the QIAGEN

Plasmid Maxi kit. The calculated coverage of these initial plasmid libraries was 353×, 6,371×, 6,015×, 8,630×, and 1,183×, of the number of oligonucleotides in each library, respectively for the Library-Profiling/ClinVar, Library-Off, Library-Small with existing scaffold, Library-Small with optimized scaffold and hMLHIdn, respectively.

PE Variant-encoding Plasmid Construction

pLenti-PE2-BSD (Addgene #161514) and pLenti-NG-PE2-BSD (Addgene #176933) plasmids were used to evaluate the prime editing efficiency induced by PE2 and NG-PE2, respectively. To generate plasmids encoding other PE variants, the pLenti-PE2-BSD plasmids were digested with XbaI and EcoRI restriction enzymes (NEB) and treated with 1 μL of Quick CIP for 10 minutes at 37° C. PE2max-encoding and Cas9-NRCH-encoding sequences, obtained from pCMV-PEmax-P2A-BSD (Addgene #174821) and pCMV-Cas9-NRCH (Addgene #136926), respectively, were amplified by PCR using Phusion High-Fidelity DNA Polymerase. The generated amplicons and the digested pLenti-PE2-BSD backbone vectors were separated through 1% or 2% agarose gel electrophoresis. Purification was performed using MEGAquick-spin™ Plus Total Fragment DNA Purification Kit (iNtRON Biotechnology, 17290). Assembly was performed using NEBuilder HiFi DNA Assembly Master Mix (NEB, E2621L) according to the manufacturer's protocol. The assembled plasmids encoding PE2max, NRCH-PE2, and NRCH-PE2max were called pLenti-PE2max-BSD, pLenti-NRCH-PE2-BSD, and pLenti-NRCH-PE2max, respectively.

### Lentivirus Creation

HEK293T cells were seeded in 100-mm or 150-mm cell culture dishes (55,000 cells/cm²) containing DMEM. After 15 hours, the DMEM was replaced with fresh medium containing 25 μM chloroquine diphosphate, and the cells were cultured for up to 5 hours. Transfer plasmids, psPAX2 (Addgene #12260) and pMD2. G (Addgene #12259) were mixed at a weight ratio of 4:3:1 and co-transfected into HEK293T cells using PEI MAX (Polysciences). 15 hours after the transfection, the culture medium was replaced with a fresh maintenance medium. At 48 hours after the transfection, lentivirus-containing supernatants were collected, filtered through Millex-HV 0.45-μm low protein binding membrane (Millipore), aliquoted, and stored at −80° C. To determine a virus titer, serial dilutions of virus aliquots were transduced into cells in the presence of polybrene (8 μg/mL). Both the cells non-transduced and cells transduced with the serially diluted viruses were maintained in the presence of puromycin (Invitrogen). When almost all the non-transduced cells were dead, the number of surviving cells was counted, and the viral titer was estimated as previously described (Shalem et al., 2014).

### PE2-Expressing and PE Variant-Expressing Cell Line Construction

The present inventors adopted PE2-expressing HCT116 and MDA-MB-231 cell lines produced in a previous study. To generate PE2-expressing or PE variant-expressing HEK293T, HeLa, DLD1, A549, and NIH3T3 cells, PE2-encoding or PE variant-encoding lentiviruses were transduced into cells at an MOI of 0.3 with 0.8 μg/mL of polybrene. 24 to 48 hours after the transduction, non-transduced cells were removed using 10 μg/mL BSD. After the BSD selection, lentiviral transmission of the PE2-encoding and PE variant-encoding sequences was confirmed using PCR and Sanger sequencing. Each cell line was continuously maintained with 10 μg/mL BSD.

### High-Throughput Evaluation of PE2, PE2max, NRCH-PE2, and NRCH-PE2max

Twenty-four hours before the lentiviral plasmid library transduction, PE2-expressing or PE variant-expressing cells were seeded in 150-mm culture dishes. Next, PE2-expressing or PE-variant expressing cells were transduced with the pegRNA-targeted pairwise library at an MOI of 0.5 with 8 μg/mL of polybrene. To achieve coverage of 500 times or more the number of pegRNA-target sequence pairs for Library-Profiling and Library-ClinVar, respectively, a total of 6×10⁸ cells were used; To achieve 2,000× coverage for Library-Small, Library-epegRNA, and Library-Off, a total of 2.4×10⁶ cells were used for Library-Small and Library-epegRNA, and 2×10⁸ cells were used for Library-Off. Twelve hours after the transduction, the culture medium was replaced with DMEM containing 10% FBS and 2 μg/mL puromycin. The cells were harvested on day 7 after the transduction for the Library-Small and Library-epegRNA, day 8 after the transduction for the Library-Profiling and Library-ClinVar, and day 10 after the transduction for the Library-Off.

### High-Throughput Evaluation of PE4max and NRCH-PE4max Systems

To evaluate high-throughput of PE4max-induced and NRCH-PE4max-induced editing efficiency in HEK293T cells using Library-Small, the present inventors delivered hMLH1dn using transient transfection of hMLH1dn encoding plasmids. pEGIP plasmids (Addgene #26777) were digested with EcoRV (NEB), and the eGFP-encoding and hMLH1dn-encoding sequences obtained from pEGIP and pEFla-hMLH1dn, respectively, were amplified by PCR. These linearized plasmids and the two inserts were Gibson assembled to prepare pLenti-EF1a-hMLH1dn-eGFP (Addgene #191104). For high-throughput experiments, 3.6×10⁷ HEK293T cells expressing PE2max were seeded in three 150-mm culture dishes and transfected with 30 μg of pLenti-hMLHIdn-eGFP plasmids using PEI. Twelve hours after the transfection, the cells were infected with Library-Small using 8 μg/mL polybrene at an MOI of 0.5. The cells were selected using puromycin and harvested on day 7 after the Library-Small transduction.

To evaluate PE4max-induced and NRCH-PE4max-induced editing efficiency in DLD1, A549, and NIH3T3 cells using Library-Small, hMLH1dn was delivered to the cells using lentiviral vectors. pLenti-gRNA Puro was cleaved using BsiWI-HF (NEB) and assembled with the MLH1dn-coding sequences amplified from pEF1a-hMLH1dn (Addgene #174824) using Gibson assembly. To suppress premature transcription termination, a silent mutation was introduced at location 134 of hMLH1dn to disrupt an AATAAA signal sequence. The resulting plasmids, named pLenti-gRNA-hMLHIdn-Puro, were digested with BsmBI enzyme at 55° C. for 6 hours and used for the construction of Library-Small-hMLH1dn as described in the “Plasmid library construction” section. Twenty-four hours before the transduction, PE2max-expressing or NRCH-PE2max-expressing cells were seeded in 150-mm culture dishes. The cells were transduced with Library-Small-hMLH1dn at an MOI of 0.5 with 8 μg/mL of polybrene. Forty-eight hours after the transduction, the medium was replaced with a fresh medium containing puromycin. The cells were harvested on day 7 after the transduction, and deep sequencing was performed.

To evaluate the PE4 system using Library-Off, hMLHIdn was delivered to HEK293T, DLD1, A549, and NIH3T3 cells using lentiviral vectors or engineered virus-like particles (eVLPs). To generate lentiviral vectors, pLenti-EF1a-hMLH1dn-eGFP was digested using Agel and Mlul and ligated with hygromycin B resistance gene (Hygro)-P2A-hMLHIdn. The resulting plasmids, named pLenti-EF1a-hMLH1dn-Hygro, were used for lentivirus production. Next, PE2max-expressing HEK293T, DLD1, and A549 cell lines were transduced with hMLH1dn-P2A-Hygro-encoding lentivirus, and then cells were selected in 100 μg/mL hygromycin. To deliver eVLP-mediated hMLH1dn, pCMV-MMLVgag-3×NES-Cas9 (Addgene #181752) was used as a backbone vector and the hMLH1dn-coding sequence of pLenti-EF1a-hMLH1dn-Hygro was inserted to generate plasmids named pCMV-MMLV-gag-hMLH1dn. To generate eVLPs containing hMLH1dn, the HEK293T cells were seeded in 150-mm culture dishes (1.2×10⁷ cells/dish) and incubated for 16-20 hours. Next, pCMV-VSV-G (Addgene #8454), pBS-CMV-gagpol (Addgene #35614), and pCMV-MMLV-gag-hMLH1dn plasmids were mixed at a ratio of 8.2:73.5:18.4 to prepare a total of 25 μg of plasmid mixture. The plasmid mixture was transfected into the cells using Lipofectamine 2000 according to the manufacturer's instructions. Six hours after the transfection, the culture medium was replaced with 20 mL of fresh DMEM, and after a further 40 hours, the medium containing eVLPs was harvested and centrifuged at 600 g for 5 minutes to remove cell debris. Next, the supernatant containing hMLH1dn-eVLP was collected and stored at 4° C. To deliver eVLP-mediated hMLH1dn, the PE2max-expressing HEK293T, DLD1, A549, and NIH3T3 cells were transduced using 7 to 10 mL of hMLHIdn-eVLP.

pegRNA Design to Evaluate Effect of Edit Types on Prime Editing Efficiency

To determine the effect of edit types in the endogenous region on prime editing efficiency (FIG. 9E), thirteen pegRNAs with high measured efficiency (15%-35%) were selected to generate pathogenic/likely pathogenic 1-bp substitutions from the ClinVar_Train dataset. For each 1-bp substitution-derived pegRNA, two additional pegRNAs with the same RHA length were generated. These two pegRNAs were designed to introduce 1-bp insertions or deletions at the same location in their target region.

Rational Design of pegRNAs

A rational design of pegRNAs for ClinVar mutation

modeling was performed as follows.

1) Find NGG PAM within +/−60 nt of the locations of intended edits, and then design all possible pegRNAs using spacers determined by the PAM. A maximum RTT length is 40 nt.

2) Select only those pegRNAs with a DeepSpCas9 score ≥30, based on spacers of the pegRNAs. When there are no pegRNAs with a DeepSpCas9 score of 30 or higher among all pegRNAs, only one spacer with the highest DeepSpCas9 score is selected among all spacers, and then only pegRNA made from that spacer is selected.

3) Select only the location of edits +5 or +6 in the pegRNAs selected in the Step 2. If not, skip this step.

4) Select pegRNAs in which the last template nucleotide is “C”, and the shortest RTT is present among the pegRNAs, which are selected in the step 3 and have RHAs with a length of 7 or more. When the RHA lengths of all pegRNAs are 6 or less, select pegRNAs with a RHA with the longest length.

5) Select pegRNAs with PBS with a length of 11, when the length of the RTT selected in the step 4 is 12 or less, meanwhile select pegRNAs with PBS with a length of 12 when the length of the RTT is 13 or more.

### Selection of Off-Target Region Candidates

To examine off-target effects associated with PE2max-based generation and modification of pathogenic mutations reported in ClinVar, the present inventors evaluated efficiency of 288, 793 pegRNAs and identified 45,691 pegRNAs with prime editing efficiency of >5%. To identify potential off-target regions for these 45, 691 pegRNAs, the present inventors identified 3,625, 682 pairs of pegRNAs and potential off-target regions using Cas-OFFinder, which allowed up to three mismatches within the guide sequences. The present inventors predicted the prime editing efficiency on these 3, 625, 682 pairs using DeepPrime-Off. Out of these, the majority (3, 196, 758, 88%) were expected to have no off-target effects. The present inventors selected 19 pairs predicted to induce prime editing effects with a frequency of >0% and selected 26 pairs predicted not to induce the prime editing effects. As described below, the present inventors performed individual prime editing experiments in endogenous regions to evaluate whether off-target effects occurred.

### Prime Editing in Endogenous Regions

To measure prime editing efficiency in endogenous regions, sequences encoding a total of 77 pegRNAs with optimized pegRNA scaffolds were cloned into pU6-pegRNA-GG-acceptor (Addgene #132777). To determine effects of edit types (FIG. 9E) and evaluate editing efficiency in BRCA2 (FIG. 6F), HEK293T cells were seeded in 48-well plates at a density of 5.0×10⁴ cells per well 22 hours prior. The cells were transfected with a mixture of plasmids encoding PE2max (pLenti-EF1a-PE2max-BSD, 300 ng) and plasmids encoding pegRNA (100 ng) using 0.8 μL of Lipofectamine 3000 and 0.6 μL of P3000 reagent, according to the manufacturer's instructions. After overnight incubation, the culture media were replaced with DMEM containing puromycin (2 μg mL⁻¹). The cells were harvested on day 7 after the transfection. To determine the frequency of off-target prime editing (FIG. 14G), the HEK293T cells were seeded in 48-well plates at a density of 1.0×10⁵ cells per well 22 hours prior. Plasmids encoding PE2max and pegRNA were delivered as described above, and the cells were harvested 6 days after the transfection.

### Deep Sequencing

To analyze prime editing efficiency in high-throughput experiments, genomic DNA was extracted from the harvested cells using the Wizard genomic DNA Purification kit (Promega). For Library-Profiling and Library-ClinVar, 5, 760 μg of genomic DNA from the HEK293T cells was used for PCR to achieve coverage greater than 960×. This assumed that there was 10 μg of genomic DNA per 10⁶ cells. A total of 576 independent 50-μL PCR reactions were performed using 200 nM of each primer set, 10 μg genomic DNA, and 25 μL 2×Taq PCR Smart mix (SolGent) under the following conditions: 22 cycles of 10 minutes at 95° C., 30 seconds at 95° C., 30 seconds at 60° C., and 40 seconds at 72° C., followed by final elongation at 72° C. for 5 minutes. To generate coverage greater than 2,000× for both Library-Small and Library-Off, PCR was performed for each sample using a minimum of 120 μg and 1 mg of genomic DNA, respectively. A total of 24 and 200 independent 50-μL PCR reactions were performed for Library-Small and Library-Off, respectively. 5 μg of genomic DNA, 500 nM of each primer set, 200 μM of dNTPs, DNA polymerase, and reaction buffer were used. For Library-Small and Library-Off, 1-2 U of Q5 High-Fidelity DNA Polymerase and Phusion DNA Polymerase were used under the following conditions: 25 cycles of 5 minutes at 98° C., then 30 seconds at 98° C., 30 seconds at 57° C. or 60° C., and 40 seconds at 72° C. in the case of Q5 High-Fidelity DNA polymerase, followed by final elongation at 72° C. for 5 minutes. 25 cycles of 10 minutes at 95° C., then 30 seconds at 95° C., 30 seconds at 57° C., and 40 seconds at 72° C. in the case of Phusion DNA polymerase, followed by final elongation at 72° C. for 5 minutes. PCR products were collected, gel purified using the MEGAquick-spin Total Fragment DNA Purification Kit (iNtRON Biotechnology), and then a sequence analysis was performed using NovaSeq (Illumina).

To measure prime editing efficiency in the endogenous regions (FIG. 9E and FIG. 6F), the cells were lysed in 100 μL of lysis buffer (10 mM Tris-HCl, pH 7.0, 0.05% SDS, and 25 μg/mL proteinase K) at 37° C. for 1 hour. The lysate was then incubated at 80° C. for 15 minutes to denature the enzyme. The first PCR was performed in a reaction volume of 50 μL using 25 μL of 2×Taq PCR Smart mix, 5 μL of cell lysate and 200 nM primer set under the following conditions: 35 cycles of 1 minute at 95° C., then 30 seconds at 95° C., 30 seconds at 60° C., and 30 seconds at 72° C., followed by final elongation at 72° C. for 2 minutes. To add Illumina adapter sequences, a second PCR was performed in a total reaction volume of 50 μL using 0.5 μL of first PCR product, 25 μL of 2×Taq PCR Smart mix, and 200 nM primer set under the following conditions: 12 cycles of 1 minute at 95° C., then 30 seconds at 95° C., 30 seconds at 60° C., and 30 seconds at 72° C., followed by final elongation at 72° C. for 2 minutes. To analyze potential off-target regions (FIG. 14G), genomic DNA was extracted from the harvested cells using the Wizard genomic DNA purification kit (Promega). Then, the first PCR reaction was performed with 80-100 ng of genomic DNA, 200 nM primer set, and Q5 High-Fidelity DNA Polymerase (NEB), under the following conditions: 12 cycles of 1 minute at 98° C., then 30 seconds at 98° C., 30 seconds at 60° C., and 30 seconds at 72° C., followed by final elongation at 72° C. for 2 minutes. For the second PCR reaction, 2 μL of the first PCR product was amplified using Q5 High-Fidelity DNA Polymerase and a 200 nM indexing primer set in a total reaction volume of 50 μL under the following conditions: 12 cycles of 1 minute at 98° C., then 30 seconds at 98° C., 30 seconds at 60° C., and 30 seconds at 72° C., followed by final elongation at 72° C. for 2 minutes. The products of the second PCR reaction were gel purified and subjected to deep sequencing. The pegRNA-coding regions, integration barcodes, and target sequences were PCR amplified from genomic DNA using Illumina adapters and PCR primers containing unique i7 and i5 barcodes.

### Prime Editing Efficiency Analysis

To analyze deep sequencing data, the present inventors used a self-made Python script adapted and extended from a previous study (Kim et al., 2021). Each pair of pegRNAs and target sequences was confirmed through 36-nt sequences (where the 36-nt sequences were made from a 12-nt sequence containing a PBS domain of pegRNA+an 18-nt barcode+a 6-nt sequence containing a neighboring sequence of 4-nt 5′ target sequence and 2-nt 5 target sequence). The sequence pairs were considered to represent PE2-induced mutations when reads showed the presence of the predetermined edits without unintended mutations within the wide target sequences. To exclude the background prime editing frequency that occurred during the array synthesis and PCR amplification process, the observed prime editing frequency was normalized using background prime editing frequency determined in the absence of PE2, as shown below.

$$= \frac{\begin{matrix}Read count of intented edits and predetermined barcodes - \\ ( Total read count of predetermined barcodes \times \\ Background prime editing frequency ) \div 100\end{matrix}}{\begin{matrix}Total read count of predetermined barcodes - \\ ( Total read count of predetermined barcodes \times \\ Background prime editing frequency ) \div 100\end{matrix}} \times 100$$

Accuracy of the analysis was improved by filtering deep sequencing data. When the pairs of the pegRNAs and target sequences were found to have a deep sequencing read count of less than 200 or a background prime-editing frequency of 5% or more, they were excluded as previously reported (Kim et al., 2021). Filtering steps were applied to reduce noise in the results due to random errors. First, high-throughput sequencing reads were classified as WT or edited reads depending on barcodes. WT reads showing random errors or edited reads showing variants other than the expected intended edits were then removed. All WT and edited reads with a total of less than 200 were removed from the study. Only those samples with a prime editing background frequency of less than 5% in those samples non-treated with PE2 were considered. For Library-Clinvar, -Profiling, -Small, -epegRNA, and -Off, high-throughput sequencing read counts of barcode-sorted clones were combined, and PE efficiency data were obtained.

### Prime Editing Efficiency Analysis in Off-Target Sequences

To analyze edits in off-target sequences, the present inventors separated sequences with edits into three groups: i) incomplete edits only of the intended edits, ii) incomplete edits only of edits at mismatched nucleotides in the corresponding RTT regions, and iii) complete edits containing both. Any changes in unintended target regions might not be good for prime editing. Thus, the efficiency of complete edits was analyzed in conjunction with the frequency of the two types of incomplete edits to determine the efficiency of off-targeted prime editing. Additionally, the present inventors evaluated all types of edits within the target cDNA regions corresponding to the pegRNAs to determine whether the intended edits were properly introduced and how prevalent the unintended edits were.

### Data Processing for Machine Learning

Data processing for machine learning, the present inventors calculated Tm, GC number, GC content, minimum free energy, and DeepSpCas9 score using biopython (1.79), ViennaRNA package (2.5.0), and DeepSpCas9 (Kim et al., 2019). These features were created by a custom Python script, accounting for the type, location, and lengths of intended edits, as well as other sequence-based features, including PBS, RTT, RTT-PBS, and RHA lengths. Each dataset was designed to ensure that there was no overlap between target sequences within the training dataset or test dataset for model development by separating each dataset into the training dataset and test dataset through stratified random sampling.

### Development of Existing Machine Learning-Based Models

To compare machine learning models trained on the ClinVar_Test dataset, the present inventors used the Pycaret package (Ali, 2020) to create Lasso, Ridge, ElasticNet, Huber, random forest, gradient boosting, XGboost, CatBoost, and LightGBM regression models. For model training, location-dependent and location-independent nucleotides and dinucleotides were extracted from the wide target, PBS, and RTT sequences. Additionally, Tm, GC number, GC content, minimum free energy, and DeepSpCas9 score normalized to z-score were included. In total, a total of 2,956 features were used to train the existing machine learning-based models. Parameters were optimized by searching 150 models using a random grid search. Spearman's correlation coefficients between measured and predicted efficiency were used as an evaluation index. To compare performance, the present inventors performed 5-fold cross-validation and compared Spearman's correlation coefficients in each validation.

### Development of DeepPrime

DeepPrime was developed as a deep learning-based computation model to predict prime editing efficiency in all target genes induced by each of pegRNAs with variable PBS and RTT lengths. The DeepPrime was designed to introduce 1-bp to 3-bp substitutions, insertions, or deletions at locations +1 to +30. The DeepPrime was implemented in PyTorch and utilized pairs of unedited (WT) and prime-edited sequences as input. Input sequence processing modules were made from four convolutional layers and a gated recurrent unit (GRU) layer. Each convolution layer was provided by using a kernel with a width of 3 and a stride of 1. There was zero padding on both ends to preserve length. There were 128, 108, 108, and 128 channels for each of the four convolutional layers, respectively. Mean pooling was performed after the 2nd, 3rd, and 4th convolution operations. Kernel size 2 and stride 2 were used for the pooling operation. The input sequences were one-hot encoded with four channels (i.e., A, T, G, C) and fed into the convolution module. Subsequently, outputs from the final convolutional layers were fed into a bidirectional GRU to train long-range interactions while preserving the locational features of the input sequences. The GRU hidden state was 128-dimensional, and the final hidden state was linearly projected into 12-dimensional vectors. Additionally, the DeepPrime has a separate four-layer recognition module for analyzing the physicochemical properties (where the physicochemical properties include Tm, GC number, GC content, minimum self-folding free energy of guide, and RTT-PBS, and DeepSpCas9 scores) of pegRNAs and target sequences, called “biofeatures”. As a result, 128-dimensional latent vectors were extracted and connected to the 12-dimensional GRU outputs to create 140-dimensional vectors. Lastly, the corresponding vectors were linearly projected to yield a single regression floating point value via softplus. For the convolution layer, the present inventors used Gaussian error linear units (GELU) activation. For other layers, rectified error linear units (ReLU) were used. Additionally, to accelerate model training, batch normalization was applied after each convolution and before the final linear projection. All hyperparameters (e.g., hidden dimension, number of layers, kernel size, stride, number of channels, training rate, and number of epochs) were optimized through Bayesian search in Optuna. To train the models, the present inventors used the AdamW optimizer and cosine annealing of the training rate with a warm restart. Five models were trained independently with different random seeds, and their predictions were averaged to obtain the final prediction. optimal hyperparameters of DeepPrime were as follows:

TABLE 1

| Optimizer | | | | Scheduler | | Model | |
|---|---|---|---|---|---|---|---|
| Batch | Training | Weight | Epoch | | T_ | Hidden | Model |
| size | rate | decay | number | T_0 | mult | size | number |
| 2048 | 5.E−03 | 5.E−02 | 10 | 10 | 1 | 128 | 5 |

### Addressing Imbalance in Data Representativeness

In the training dataset, there was a high proportion of data with low PE efficiency. This limited the representativeness of cases with high PE efficiency. To address this imbalance issue, high offset coefficients were used to minimize the loss of underrepresented data, allowing the models to be trained more sensitively to rare data. A multiplied coefficient (μ) was obtained by approximating reciprocal of the square root of the underrepresented data using a simple function, as shown below.

$$μ = min ( exp ( 6 ( log ( x + 1 ) - 3 ) + 1 ) , 5 ) = min ( \frac{{( x + 1 )}^{6}}{exp ( 18 )} + 1 , 5 )$$

Herein, x is the measured prime editing efficiency (%).

Additionally, to address certain data imbalances within the type of edits, values for losses corresponding to insertions and deletions, which were relatively rare compared to substitutions, were multiplied by weights of 0.7 and 0.6, respectively. Weights applied to the insertions and deletions were determined by analysis using 5-fold cross-validation.

### Development of DeepPrime-FT

Transfer training was applied for fine-tuning DeepPrime, a basic model. Eighteen models were created by fine-tuning with 18 datasets on prime editing efficiencies induced by eight different prime editing systems containing two types of scaffold sequences in seven different cell types. Final weights of DeepPrime were used as the initial weights for these models. The batch size was set to 512 for all fine-tuned models. Optimal hyperparameters, including training rate, weight decay coefficient, and number of epochs, were determined using Optuna. The optimal hyperparameters for the 18 models are as follows:

TABLE 2

| | | | | | | Scheduler | | | |
|---|---|---|---|---|---|---|---|---|---|
| | | | | | | T_0 | | | |
| | | Optimizer | | | | (when | | Model | |
| Cell | PE | Batch | Training | Weight | Epoch | using | T_ | Hidden | Model |
| line | system | size | rate | decay | number | scheduler) | mult | size | number |
| A549 | PE2max | 512 | 1.E−02 | 2.E−02 | 40 | 20 | 1 | 128 | 20 |
| A549 | PE2max- | 512 | 2.E−03 | 1.E−02 | 100 | — | — | 128 | 20 |
| | e | | | | | | | | |
| A549 | PE4max | 512 | 5.E−03 | 2.E−02 | 50 | 25 | 1 | 128 | 20 |
| A549 | PE4max- | 512 | 1.E−02 | 2.E−02 | 100 | 50 | 1 | 128 | 20 |
| | e | | | | | | | | |
| DLD1 | NRCH- | 512 | 4.E−03 | 2.E−02 | 100 | — | — | 128 | 20 |
| | PE4max | | | | | | | | |
| DLD1 | PE2max | 512 | 2.E−03 | 2.E−02 | 100 | — | — | 128 | 20 |
| DLD1 | PE4max | 512 | 1.E−03 | 0.E+00 | 100 | — | — | 128 | 20 |
| HCT116 | PE2 | 512 | 8.E−03 | 1.E−02 | 50 | — | — | 128 | 20 |
| HEK293T | NRCH- | 512 | 1.E−02 | 1.E−02 | 50 | — | — | 128 | 20 |
| | PE2 | | | | | | | | |
| HEK293T | NRCH- | 512 | 4.E−03 | 1.E−02 | 50 | — | — | 128 | 20 |
| | PE2max | | | | | | | | |
| HEK293T | PE2 | 512 | 2.E−03 | 1.E−02 | 100 | — | — | 128 | 20 |
| HEK293T | PE2max | 512 | 1.E−03 | 0.E+00 | 100 | — | — | 128 | 20 |
| HEK293T | PE2max- | 512 | 1.E−02 | 1.E−02 | 100 | 50 | 1 | 128 | 20 |
| | e | | | | | | | | |
| HEK293T | PE4max | 512 | 5.E−03 | 1.E−02 | 100 | — | — | 128 | 20 |
| HEK293T | PE4max- | 512 | 5.E−03 | 1.E−02 | 50 | — | — | 128 | 20 |
| | e | | | | | | | | |
| HeLa | PE2max | 512 | 1.E−02 | 2.E−02 | 50 | 25 | 1 | 128 | 20 |
| MDA- | PE2 | 512 | 5.E−03 | 1.E−02 | 100 | — | — | 128 | 20 |
| MB-231 | | | | | | | | | |
| NIH3T3 | NRCH- | 512 | 2.E−03 | 2.E−02 | 100 | — | — | 128 | 20 |
| | PE4max | | | | | | | | |

### Development of DeepPrime-Off

DeepPrime-Off was developed by fine-tuning DeepPrime, as described in the “Development of DeepPrime-FT” section. To train DeepPrime-Off, the present inventors used the AdamW optimizer and cosine annealing of the training rate with a warm restart. Additionally, sequence information on pairs of off-targets and pegRNA sequences was added as input to account for the interactions between them. Additionally, data shortage and bias issues were addressed using data augmentation and loss weighting.

### Off-Targeted Data Augmentation

To overcome the limitations of data diversity, the present inventors applied two new data augmentation techniques based on the following observations obtained from off-target profiling. First, it was found that editing efficiency converged to 0% when there were 6 or more sequence mismatches. Accordingly, the data was augmented by 5% by introducing corrected zero-label data points such that the target sequences showed a 40% mismatch with the guide sequences. Additionally, it was found that sequences outside the region corresponding to the pegRNA-target DNA interaction did not affect PE efficiency. Therefore, data augmentation techniques that added random mutations to non-PE interaction regions were used.

### Off-Target Loss Weighting

A significant portion of the Library-Off data contained pegRNAs with an edit location of +5, so the explanatory ability of data, whose edit location was not +5, might be limited and low. To address this issue, the present inventors multiplied the loss by 0.25, which was a relatively small weight value when the edit location was +5. This alleviated the problem of reduced regression errors in different edit locations due to lack of representativeness. The hyperparameters used during DeepPrime-Off training were batch size of 256, training rate of 4×10⁻², weight decay of 1×10⁻², and number of epochs of 5. These hyperparameters were all determined using Optuna. Five models were trained independently, and their predictions were averaged into the final prediction score. DeepPrime-Off's optimal hyperparameters were as follows:

TABLE 3

| | | |
|---|---|---|
| Optimizer | Batch size | 256 |
| | Training rate | 4.E−02 |
| | Weight decay | 1.E−02 |
| | Epoch number | 5 |
| Scheduler | T_0 | 5 |
| | T_mult | 1 |
| Model | Hidden size | 128 |
| | Model number | 5 |
| Augmentation | Off-target mutation rate (transformed | 0.05 |
| | portion of data to dummy data with 0% | |
| | off-target efficiency) | |
| | Targeting (mutating nucleotides at non- | 1 |
| | interacting regions in the target DNA) | |

### Interpretation and Feature Analysis of Tree-Based Machine Learning Models

To quantify the contribution of each feature to a model the present inventors analyzed predicting PE efficiency, Shapley values using the Shapley Additive explanations (SHAP, 0.40.0) Python package. A light gradient boosting machine (LightGBM) model was trained in the same manner as described above, with or without removing associated features with correlation coefficients of 0.7 or higher. To determine the global contribution of each feature to the predictive model, local interaction effects were measured by using the SHAP values of the entire training dataset and comparing the Shapley values for each feature.

### Quantification and Statistical Analysis

To compare prime editing efficiency between experiments using different pegRNAs, the present inventors used one-way analysis of variance (ANOVA) and two-sided Türkiye's post hoc test. To compare the Spearman's correlations between the prediction scores generated by the predictive model (FIG. 3B and FIG. 3C), a two-sided Steiger test was used. This method was intended to test two dependent correlation coefficients in exactly the same dataset. To determine statistical significance, GraphPad Prism 8, PASW Statistics (version 17.0, IBM), and Microsoft Excel (version 16.0, Microsoft Corporation) were used. To perform high-throughput evaluation of PE2 efficiency using pairwise libraries, high-throughput sequencing read counts obtained from two replicates transfected independently by two different experimenters were combined.

### Results

### High-Throughput Evaluation of PE2 Efficiency Performed Using Five Pairwise Libraries

To evaluate the high-throughput of PE2 efficiency, the present inventors delivered lentiviral pairwise libraries of pegRNA-encoding sequences and corresponding target sequences (FIG. 1A and FIG. 8A) into PE2-expressing HEK293T cells. The present inventors prepared five pairwise libraries and named them Library-Profiling, Library-ClinVar, Library-Small, Library-epegRNA, and Library-Off, respectively (Method, FIG. 8B). Prime editor-expressing HEK293T cell lines were transduced with one of these libraries, and editing efficiency was determined by deep sequencing. Two independent replicates showed a strong correlation (Pearson's correlation coefficients (r)=0.90 and 0.92, Spearman's correlation coefficients (R)=0.94 and 0.92 for Library-Profiling and Library-ClinVar) (FIG. 8C).

### Analysis of Factors Affecting Prime Editing Efficiency

When determining the effect of a length of PBS on editing efficiency using pegRNAs containing RTTs with a length of 12-nt and 20-nt, respectively, the highest mean efficiency was observed for PBSs with a length of 11-nt (mean efficiency, 13%) and 12-nt (8.5%), respectively (FIG. 1B). This result was consistent with our previous findings, although the PBS with a length of 12-nt was not tested in a previous study (Kim et al., 2021). A similar trend was observed when a similar analysis was performed using Library-ClinVar (FIG. 9A). On the basis of these results, the present inventors suggest using the PBS with a length of 11-nt when the length of the RTT is 12 nt or less, and using the PBS with a length of 12-nt when the length of the RTT exceeds 12 nt. Next, the present inventors evaluated the effect of RTT lengths and found that the most efficient mean prime editing efficiency was observed when using the RTT with a length of 12-+2-nt (FIG. 1C). Observations in the analysis using Library-ClinVar (FIG. 9A) were consistent with the previous analysis (Kim et al., 2021).

When determining the effect of locations of edits, mean edit efficiency decreased sharply from locations approximately nts before the end of the RTT (e.g., when the length of RTT 5 was 12 nts, the sharp decrease in efficiency was at +7 (=12 nts−5 nts)) (FIG. 1D). This suggests that there was a minimum length requirement for the right homology arm (RHA), i.e., the right location of the RTT shown in FIG. 8A. Unlike the PBS and RTT lengths, the significance of the RHA lengths has not been analyzed extensively. During the prime editing process, when the RHA is too short, there may be a strong preference for equalization for the 3′ flap rather than the 5′ flap. Of these, the latter is required to integrate edited sequences into the genome (FIG. 9B). When determining the effect of the RHA lengths using pegRNAs different locations and types of edits, regardless of the locations of the edits, RHAs with a length of 5-nt, 7-nt, and 9-nt were required for substitution, insertion, and deletion, respectively (FIG. 1E-G). Additionally, similar RHA requirements were observed in analyses using Library-ClinVar (FIG. 9C). However, to summarize these results in a nutshell, using RHA with a length of 9-nt or at least 7-nt for all types and locations of edits is recommended. Additionally, the overall prime editing efficiency for substitutions was slightly higher than that for insertions and deletions (FIG. 9C and FIG. 9D). A similar weak trend, although not statistically significant, was observed in prime editing of the endogenous regions (FIG. 9E).

Then, the effect of the edited nucleotide number on prime editing was evaluated. As the edited nucleotide number increased, the efficiency was similar for substitutions up to 3 bp and decreased for 4-bp to 10-bp substitutions (FIG. 1H). Efficiency was similar for insertions and deletions up to about 3 to 5 bps. Thereafter, efficiency decreased as the length of the inserted or deleted sequence increased (FIGS. 1I and 1J). These results show that PE2 efficiency tended to decrease as the edited nucleotide number increased, especially for more than 3 nucleotides. Additionally, it was found that the preferred nucleotide at the last template location for efficient prime editing was in the order of C>T>A>G, regardless of the types of edits type and the length of the RTT (FIG. 1K, FIG. 9D, and FIG. 9F).

In addition, SHapley Additive explanations (SHAP) analysis was performed with and without considering multicollinearity to identify the determinants of prime editing efficiency (FIG. 2A and FIG. 10A). The most important feature was a GC number in the PBS (preferred). This was associated with the PBS length (complex, too high a value was not preferred), the melting temperature of the PBS (Tm), the GC content of the PBS, the number of Cs in the PBS (preferred), and the number of Gs in the PBS (preferred). The second most important features were the RHA length (preferred when multicollinearity was not considered) and the Im of the RHA (preferred when multicollinearity was considered). Both were associated with the GC content of the RHA (low values were unfavorable; medium to low values were preferred; significantly high values were significantly unfavorable) and the GC number of the RHA (low values were significantly unfavorable, medium to high values were significantly preferred, and significantly high values were only slightly preferred). The third important feature was the DeepSpCas9 score (preferred) on the corresponding target sequences, which was consistent with the previous findings (Kim et al., 2021). The present inventors determined the overall optimal range for each important feature but also found that the optimal range of values for each feature often varied depending on the values of other features (FIGS. 2B-2G, and FIG. 10B-G). This made it difficult to manually design efficient pegRNAs.

### Development of DeepPrime

The predictive models for PE2 activity, which were DeepPE, PE_type, and PE_position, previously developed by the present inventors had limited scope with respect to edit types, locations of edits, and PBS and RTT lengths, mainly due to insufficient training datasets (Kim et al., 2021). The prime editing efficiency dataset generated with Library-ClinVar contained information on 288, 793 pairs of pegRNA-encoding sequences and target sequences with 850 (=17×50) combinations of the PBS and RTT lengths for all edit types containing 1, 2, or 3 bps, including substitutions, insertions and deletions at all locations of edits from +1 to +30. These datasets were split into two datasets, ClinVar_Train (n=259, 910) and ClinVar_Test (n=28,883), by random sampling (same target sequences were not shared between the two data sets). Compared to DeepPE's training datasets, the ClinVar_Train dataset was 6.7 times larger, contained 35 times more range of PBS-RTT length combinations (FIG. 11A), 582 times more combinations of types and locations of edits (FIG. 11B), and 30 times (=52,723/1,756) more target sequences. The present inventors used ClinVar_Train as training data and compared 9 existing machine learning algorithms and 6 deep learning algorithms, thereby developing models to predict the efficiency for PE2-induced desired edits in given target sequences. When comparing the performance of these 15 algorithms using 5-fold cross-validation, performance of the algorithm based on a convolutional neural network (CNN) with a gated recurrent unit (GRU) (FIG. 3A) was significantly higher than that of the next best algorithm (CNN with an attention module) (P≤9.3×10⁻³ and 8.8×10⁻³, Steiger's test of Pearson's and Spearman's correlations) (FIG. 3B-C). Accordingly, the present inventors developed a CNN with a GRU-based computation model and named it DeepPrime. When determining how training dataset size affects model performance, it was found that performance was nearly stable when the dataset contained more than about 180,000 data points (FIG. 12A-B). However, the accuracy of prediction for deletions and 3-bp edits was relatively low. This was probably because there were relatively few data points of this kind even after augmenting the data points in the library ClinVar design (FIG. 8B).

When evaluated using ClinVar_Test, DeepPrime showed high performance with a Pearson's correlation coefficient (r) of 0.84 and a Spearman's correlation coefficient (R) of 0.86 (FIG. 3D). To determine whether DeepPrime exhibited high performance across all editing types, performance was evaluated using nine ClinVar_Test subsets representing nine types of intended edits (e.g., 1-bp, 2-bp, or 3-bp substitutions, insertions, and deletions). High Pearson's correlation coefficients (r) and Spearman's correlation coefficients (R) (r ranged from 0.76 to 0.89 (mean 0.82, median 0.81) and R ranged from 0.70 to 0.88 (mean 0.80, median 0.83)) were observed (FIG. 3E). Additionally, when confirmed how well DeepPrime performed depending on the location of the intended edits, r ranged from 0.68 to 0.85 (mean 0.79, median 0.80) at locations +1 to +30, R ranged from 0.63 to 0.88 from locations +1 to +27 and ranged from 0.47 to 0.58 at locations +28 to +30 (mean 0.75, median 0.78 at locations +1 to +30) (FIG. 3F). Taken together, these results showed that DeepPrime might accurately predict prime editing efficiency across the types, lengths, and locations of edits.

In a previous study, the present inventors performed high-throughput evaluation experiments to identify pegRNAs with the highest efficiency for any given intended edits (Jang et al., 2021). The present inventors observed a high correlation between the efficiency predicted by DeepPrime and the experimentally measured efficiency (FIG. 3G). Furthermore, among 100 possible pegRNAs for intended edits, the pegRNAs proposed by DeepPrime were identical to those proposed experimentally. Additionally, when DeepPrime was tested using PE2 efficiency data from an earlier (independent study) study of prime editing in endogenous regions (Anzalone et al., 2019), the model had high Pearson's and Spearman's correlation coefficients of r=0.74 and R=0.74, respectively. This suggests that performance of the DeepPrime in predicting PE2 efficiency in the endogenous regions was excellent (FIG. 3H).

Improvement of Prime Editing Efficiency using Optimized pegRNA Scaffolds, PAM Optimal Co-Editing, and PE Variants

The present inventors compared the prime editing efficiency of pegRNAs with existing and optimized scaffolds using Library-Small because the optimized sgRNA scaffolds with 5-nt longer loops and TTTC sequence instead of TTTT sequence improved Cas9 activity. The present inventors found that the optimized pegRNAs showed higher efficiency than conventional pegRNAs for 79% ( 1,674/2,132) of pegRNA-target sequence pairs, resulting in a statistically significant 1.25-fold increase in mean efficiency (FIG. 4A). This suggests that prime editing efficiency might often be improved by using optimized pegRNA scaffolds.

Disrupting the NGG PAM sequence through co-editing along with the intended edits might enhance the efficiency of prime editing. To examine the effect of the NGG PAM sequence editing, pegRNAs with and without PAM co-editing of all 15 possible types were used. Through this PAM co-editing, the mean prime editing efficiency increased by 1.7 to 1.2 times, and the highest mean editing efficiency was observed when the NGG PAM sequences were edited with NAT sequences (FIG. 4B). When it was necessary to induce significantly synonymous editing of the NGG PAM sequences, two or more non-GG sequences might often be used.

The absence of the NGG PAM sequences near the intended edit sites might often limit the application of efficient prime editing. To expand the range of target sequences, two PE2 variants were generated on the basis of SpCas9-NG (NG-PE2) and SpCas9-NRCH (NRCH-PE2). When comparing the mean efficiencies of prime editing and nuclease-induced indel generation, a high correlation was found across PE variants (r ranged from 0.85 to 0.97 (mean 0.93, median 0.97), R ranged from 0.75 to 0.89 (mean 0.81, median 0.78) (FIG. 13A-E). This supports the fact that Cas9 and the corresponding Cas9-based PE had similar PAM compatibility.

Next, the mean prime editing activity of PE2, NRCH-PE2, and NG-PE2 on target sequences containing 3-nt PAM sequences (NXXX, where X was changed) was determined. When the PAM sequences were defined as sequences with a mean prime editing efficiency higher than 1% at day 7 after the transduction of the pegRNA (using an optimized scaffold)-targeting library, 12 of 64 (19%), 30 of 64 (47%), and 26 of 64 (41%) 3-nt potential PAM sequences might be used as the PAM sequences by PE2, NRCH-PE2, and NG-PE2, respectively (FIG. 13F-H). When comprehensively considering the PAM sequence compatibility of the three PE2 variants, it was found that 35 (55%) of 64 potential 3-nt PAM sequences might be used as the PAM sequences by at least one of the PE2 variants (FIG. 4C-D).

Prime editing efficiency might also be improved by using recently modified prime editors, including PE2max and PE4max. The PE4max was a combination of PE2max, an improved version of the PE2, and MLH1dn, which suppressed the MMR. A high prime editing efficiency was observed correlation between between the PE2 and PE2max (r=0.91, R=0.96) and between the PE2 and PE4max (r=0.88, R=0.96). Compared to the prime editing efficiency of the PE2 in HEK293T cells, it was found that PE2max and PE4max showed 1.9-fold and 2.7-fold improved prime editing efficiency, respectively (FIG. 4E-F).

The efficiency of prime editing might vary depending on the cell type, at least in part due to different expression levels of MMR-associated components when the efficiency of each of the PE2, PE2max, PE4max, and NRCH-PE4max measured in the HEK293T cells was compared with those measured in other cell types such as HCT116, MDA-MB-231, HeLa, A549, DLD1, and NIH3T3 cells, it was found a variety of correlations (r ranged from 0.63 to 0.89 and R ranged from 0.80 to 0.93) (FIG. 4G-M). This supports the fact that prime editing efficiency might vary depending on cell types. Additionally, the previous studies have shown that the use of epegRNA increased prime editing efficiency, but the magnitude of the increase varied depending on the cell types and prime editors. That is, when tested in the HEK293T cells and A549 cells, when epegRNAs were used instead of regular pegRNAs, the mean efficiency of the PE2max increased by 1.5 times in the HEK293T cells, and the mean efficiencies of the PE2max and PE4max increased by 4.1-fold and 0.89-fold, respectively, in the A549 cells (FIG. 4N-P, FIG. 12I, and FIG. 12J).

### Development of DeepPrime-FT

When the prime editing efficiency induced by different prime editors in various types of cells might be predicted, selection of appropriate PE variants and pegRNAs for a given variety of experimental conditions would be greatly facilitated. To assist in selecting appropriate PE variants and pegRNAs to introduce given intended edits, the present inventors developed computation models to predict prime editing efficiency under various experimental conditions. Eighteen datasets on prime editing efficiency induced by PE2-, PE2max-, PE2max-e (PE2 with epegRNA), PE4max-, PE4max-e (PE4max with epegRNA), NRCH-PE2, NRCH-PE2max, or NRCH-PE4max were generated using Library-Small in seven different cell lines (HEK293T, HCT116, DLD1, MDA-MB-231, A549, HeLa, and NIH3T3). These were split into training and testing datasets. Afterward, the present inventors fine-tuned the DeepPrime using the training datasets to develop 18 different computation models, collectively named DeepPrime-FT (FIG. 5A). When the performance of the DeepPrime and DeepPrime-FT was evaluated using 19 test datasets, it was found that the DeepPrime performed well across cell types, scaffolds, and prime editors (r, mean 0.58, median 0.57, range 0.42 to 0.83; R, mean 0.73, median 0.75, range 0.51 to 0.86). It was also found that the fine-tuning further improved performance (r, mean 0.71, median 0.74, range 0.51 to 0.83; R, mean 0.80, median 0.82, range 0.57 to 0.89) (FIG. 5B and FIG. 5C). Spearman's correlation coefficients for the DeepPrime were frequently higher than Pearson's correlation coefficients across cell types, scaffolds, and Prime Editor systems. This may suggest that the ranking of pegRNA efficiencies was relatively better preserved on a linear scale compared to relative pegRNA efficiencies. Across cell types and Prime Editor systems, important features determined by the SHAP analysis were primarily shared. The effects of the factors affecting g prime editing efficiency described above were observed similarly, although not identically (data not shown). This was consistent with the good performance of the DeepPrime across cell types and PrimeEditor systems. Thus, researchers might select pegRNAs based on approximate DeepPrime-based predictions across experimental conditions, but to maximize prediction accuracy, researchers might also choose fine-tuned models that most closely reflect the experimental conditions with respect to cell type, scaffold, and Prime Editor version.

### Examples of Applications of DeepPrime and DeepPrime-FT

Next, the present inventors determined whether the DeepPrime and DeepPrime-FT could be utilized for efficient generation and correction of pathogenic and likely pathogenic mutations reported in the ClinVar (Landrum et al., 2020; Landrum et al., 2016). pegRNAs expected to have the highest efficiency for given examples of target prime editing were designed using three approaches: i) DeepPrime was used, ii) pegRNA was rationally designed using common features of high-efficiency pegRNA (see Methods), and iii) only the DeepSpCas9 scores representing the predicted Cas9 activity were used (Kim et al., 2019). This was correlated with prime editing efficiency (Kim et al., 2021). As a negative control, pegRNAs were randomly designed. On the basis of expected efficiency of the designed pegRNAs, it was found that the pegRNA design approaches were ranked as follows: for variant correction, DeepPrime (mean and median expected prime editing efficiency=9.0% and 7.6%)>>rational design (4.6% and 2.4%)>DeepSpCas9 score-based design (3.2% and 1.1%)>randomized design 1 (1.2% and 0.3%)=randomized design 2 (1.2% and 0.3%) (FIG. 6A); and for variant generation, DeepPrime (mean and median expected prime-editing efficiency=10% and 9.2%)>>rational design (5.3% and 3.1%)>DeepSpCas9 score-based design (3.8% and 1.4%)>randomized design 1 (1.4% and 0.3%)=randomized design 2 (1.4% and 0.3%) (FIG. 6B). In particular, considering that the mean and median prediction efficiencies of pegRNAs designed using DeepPrime were 2.0 and 3.2 times (for variant correction) and 1.9 and 2.9 times (for variant generation) higher than those of rationally designed pegRNAs, respectively, DeepPrime would be significantly useful for efficient pegRNA design for mutation correction or generation.

Erwood et al. recently used prime editing to generate variants in NPC1 and BRCA2. It was found that the DeepPrime-based pegRNA design could improve the mean efficiency of variant generation by 2.1-fold compared to the rational pegRNA design (FIG. 6C). Additionally, when the NRCH-PE2 and DeepPrime-FT or PE2max and DeepPrime-FT had been used, the mean prime editing efficiencies could have been 4.5-fold or 7.7-fold higher, respectively, than those obtained using a rationally designed pegRNAs with PE2. Taken together, these analysis results indicate that DeepPrime and DeepPrime-FT combined with improved PE might facilitate the functional evaluation of variants by enabling efficient generation.

When the efficiency ranking of the pegRNAs selected by the DeepPrime was determined among the eight pegRNAs for given edits in the ClinVar_Test, 50% and 25% of the efficiencies ranked first and second, respectively (FIG. 6D). When similar analyses were performed using results from previous experiments in endogenous regions, 56% and 11% ranked in the top 10% and between 10% and 20%, respectively (FIG. 6E).

The present inventors also compared prime editing efficiency in the endogenous regions. In this situation, the DeepPrime-FT predicted that the DeepPrime-FT-based pegRNA design would have a mean PE2max efficiency of 2.3 times higher than the previously published design. In experiments performed in the HEK293T cells, the DeepPrime-based pegRNA design showed a mean PE2max efficiency 3.5 times higher than previously published designs (FIG. 6F). Prime editing efficiencies measured in the endogenous regions showed a high correlation with the efficiencies predicted by the DeepPrime-FT (r=0.82 and R=0.83) (FIG. 6G). These results support that prime editing might be carried out efficiently through the DeepPrime-FT-based design.

To determine whether the DeepPrime might predict efficiencies of other untested prime editing systems, such as PE3 and PE5, in endogenous regions, evaluations were conducted using previously published data (Anzalone et al., 2019; Chen et al., 2021). The DeepPrime performed well in predicting the efficiency of PE3 and PE5, although its performance was not as good as those observed in the prime editing systems tested (PE3, r=0.65, 0.63, R=0.61, 0.59; PE5, r=0.68, R=0.64) (FIG. 6H-J). This slightly lower performance might be due to the fact that the activities of PE3 and PE5 might be affected not only by the pegRNAs but also by the sgRNAs used, whose activity was not predicted by the DeepPrime.

### High-Throughput Profiling of Prime Editing Efficiency on Off-Target Sequences

Prime editing might also occur when there were mismatches between the target sequences and pegRNAs, resulting in off-target prime editing. Accordingly, the present inventors extensively examined prime editing efficiency using a total of 47,839 pairs of pegRNAs and on-target sequences and of pegRNAs and off-target sequences in Library-Off.

First, the effect of locations of the mismatchs and the effect of mismatched nucleotide number on prime editing efficiency were examined in the HEK293T cells. It was found that the relative editing efficiency (=efficiency of pegRNAs in target sequence containing mismatches/efficiency of pegRNAs in on-target sequence) of the PE2 and PE4max decreased as the mismatch locations approached location 17. The lowest relative prime editing efficiency was observed at this location (FIG. 7A and FIG. 14A). The relative editing efficiency was high at locations 1 and 2. This was consistent with the finding that the resistance of SpCas9 nuclease to mismatches was highest at these locations (Kim et al., 2020b; Kim et al., 2020c). From location 11, the relative efficiency gradually decreased until location 15 and then decreased sharply at locations 16 and 17. The lowest resistance was observed at locations 16 and 17. This was due to the fact that mismatches at these locations, especially location 17, prevented reverse transcription, which was necessary for prime editing. Locations 18 (corresponding to location +1 of pegRNAs) to 29 correspond to the RTT domain of pegRNAs. Mismatch tolerance was relatively high at this location, as mismatches in this region could be considered additional edits. When the mismatch tolerance was evaluated for the PE2max or PE4max instead of the PE2 in two or three other cell lines, a consistent trend in mismatch tolerance was found depending on the mismatch location (FIG. 7B and FIG. 14B). Despite these similar trends, the general level of prime editing activity and specificity varied across cell types and prime editors (FIG. 7B, FIG. 7C, and FIG. 14B). Interestingly, a trade-off between the general activity and specificity of prime editing was observed. A similar trend was observed when comparing different SpCas9 variants in previous studies (Kim et al., 2020c; Schmid-Burgk et al., 2020). As the mismatch number increased, the relative prime editing efficiency decreased (FIG. 7A and FIG. 14A). Additionally, the shorter the PBS length, the lower the mismatch tolerance at locations 3 to 17 (FIG. 7D). On the other hand, the RTT length had little effect on mismatch tolerance, with the only exceptions when the mismatches were located at location 25 (location +8 of pegRNAs) and when the RTT length was 10 nt. Thus, the RHA was only two nucleotides long, which was much shorter than the RHA length typically required when the mismatches were considered additional edits (FIG. 7E).

Next, whether mismatch types affects prime editing efficiency in off-target sequences was examined. For all tested locations, a mismatch tolerance resulting in wobble and non-wobble purine-pyrimidine base pairing was slightly higher or at least similar to a mismatch tolerance resulting in purine-purine or pyrimidine-pyrimidine pairing (FIG. 7F). This was partially consistent with the results obtained in experiments using Cas12a (Kim et al., 2017) and SpCas9 (Kim et al., 2020b) in previous studies.

In the experiments described so far, the present inventors evaluated the prime editing efficiency on target sequences, where an intended G to C transversion did not occur and encoding was made at a location corresponding to the location +5 of pegRNAs. Next, the present inventors examined whether similar mismatch tolerance trends would be observed when using pegRNAs with different types of encoded edits, each of which was made at different locations. The present inventors found that the effect of mismatch locations was generally similar regardless of the types and locations of the intended edits, except when the intended edits were encoded at location +9 (corresponding to location 26 of the target sequences). In this case, considering the off-targets as additional edits, the mismatch at location 28 resulted in the length of the RHA being too short (only 1 nt) (FIG. 7G).

Next, the present inventors developed a computation model to predict prime editing efficiency in off-target sequences. The datasets containing prime editing efficiency measured using Library-Off were named PE2-Off. These were split into PE2-Off_Train (n=18,085) and PE2-Off_Test (n=4,522) by stratified random sampling (see Methods). By fine-tuning the DeepPrime using PE2-Off_Train, DeepPrime-Off (FIG. 7H) was developed. This predicted the prime editing efficiency induced by the pegRNAs in off-target sequences. When evaluated using PE2-Off_Test as the test datasets, Pearson's and Spearman's correlation coefficients between measured and predicted editing efficiencies were 0.77 and 0.70 (FIG. 7I), suggesting that performance of the DeepPrime-Off was strong. To determine whether the DeepPrime-off performed well across edit types, edit locations, and RTT lengths, performance of the DeepPrime-off was also evaluated on 11 subsets of PE2-Off_Test. These subsets represented three different types of intended edits, two different intended edit locations, and five different RTT lengths. High Pearson's (r) and Spearman's (R) correlation coefficients were observed for each type of edits (r=0.56 to 0.77 (mean 0.70), R=0.66 to 0.79 (mean 0.69)), edit location (r=0.53 to 0.78 (mean 0.66), R=0.53 to 0.82 (mean 0.68)) and each RTT length (r=0.56˜0.84 (mean 0.74), R=0.59˜0.83 (mean 0.71)) (FIG. 14C-E). This suggests good performance across these variables. Additionally, when the DeepPrime-Off was evaluated using independently determined off-target effects for 216 pairs of pegRNAs in endogenous regions in the previous study (Kim et al., 2020a), the results were r=0.93 and R=0.999 (FIG. 7J). When 190 pairs without activity were randomly removed to equalize the number of pairs with and without off-target prime editing activity (both 13), r=0.89 and R=0.92. This indicates that the DeepPrime-Off had excellent performance in predicting off-target effects in the endogenous regions.

To find potential off-target effects associated with the generation and modification of PE2max-driven pathogenic mutations, 3,571,521 pairs of pegRNAs and potential off-target regions with up to three mismatches were identified (see Methods). The DeepPrime-Off shows that 3, 183, 918 pairs (89%) of these pairs did not show any prime editing effects. The remaining 387,603 pairs (11%) were predicted to show a prime editing effect at a frequency of >0% (FIG. 14F). Of the 3,183,918 pairs predicted to have no prime-editing effect, 26 pairs had no detectable effect when randomly tested. When 19 of the 387, 603 pairs predicted to have a prime editing effect in the endogenous regions were tested, an off-target effect in 2 of the 19 pairs was observed (FIG. 14G). These data suggest that the DeepPrime-Off might be useful in reducing the number of potential off-target regions and in prioritizing those that should be tested.

The present inventors provide a web tool at http://deepcrispr.info/DeepPrime that shows the results of the DeepPrime, DeepPrime-FT, and DeepPrime-Off for given intended edits. Related Python packages are also provided at https://pypi.org/project/genet/.

### CONCLUSION

Given a single intended edit, there are often multiple potential target sequences near the desired edit regions. Additionally, theoretically, at least 850 pegRNAs (=17 PBS length×50 RTT length) can be designed per target sequence. When there are four potential target sequences, the number of pegRNAs that can be designed reaches 3,400 (=850×4). The DeepPrime and DeepPrime-FT can predict the efficiency of thousands of pegRNAs, making it possible to identify the most efficient pegRNAs in minutes without performing actual experiments.

In summary, the present inventors have generated data on prime editing efficiency on an unprecedented scale in an error-free manner. The present inventors extensively characterized the determinants of prime editing and developed computation models to predict prime editing efficiency for multiple cell types and various prime editors. The present inventors also extensively profiled prime editing efficiency at off-target sequences and developed computation models to predict prime editing efficiency in these regions. Using these models, it is possible to computationally predict the prime editing efficiency on on-targets and/or off-targets for various combinations of prime editors and pegRNAs, thereby selecting the most efficient and specific combination.

### REFERENCES

Kim, et al. (2019). SpCas9 activity prediction by DeepSpCas9, a deep learning-based model with high generalization performance. Sci. Adv. 5, eaax9249.
Landrum, et al. (2016). ClinVar: public archive of interpretations of clinically relevant variants. Nucleic Acids Res. 44, D862-868.
Kim, et al. (2020a). Unbiased investigation of specificities of prime editing systems in human cells. Nucleic Acids Res. 48, 10576-10589.
Shen, et al. (2017). Combinatorial CRISPR-Cas9 screens for de novo mapping of genetic interactions. Nat. Methods 14, 573-576.
Du, et al. (2017). Genetic interaction mapping in mammalian cells using CRISPR interference. Nat. Methods 14, 577-580.
Shalem, et al. (2014). Genome-scale CRISPR-Cas9 knockout screening in human cells. Science 343, 84-87.
Kim, et al. (2021). Predicting the efficiency of prime editing guide RNAs in human cells. Nat. Biotechnol. 39, 198-206.
Jang, et al. (2021). Application of prime editing to the correction of mutations and phenotypes in adult mice with liver and eye diseases. Nat. Biomed. Eng. 6, 181-194.
Landrum, et al. (2020). ClinVar: improvements to accessing data. Nucleic Acids Res. 48, D835-D844.
Erwood et al., (2022). Saturation variant interpretation using CRISPR prime editing. Nat Biotechnol. 2022 June; 40(6): 885-895.
Chen, et al. (2021). Enhanced prime editing systems by manipulating cellular determinants of editing outcomes. Cell 184, 5635-5652.e5629.
Kim, et al. (2020b). High-throughput analysis of the activities of xCas9, SpCas9-NG and SpCas9 at matched and mismatched target sequences in human cells. Nat. Biomed. Eng. 4, 111-124.
Kim, et al. (2020c). Prediction of the sequence-specific cleavage activity of Cas9 variants. Nat. Biotechnol. 38, 1328-1336.
Schmid-Burgk, et al. (2020). Highly Parallel Profiling of Cas9 Variant Specificity. Mol. Cell 78, 794-800 e798.
Kim, et al. (2017). In vivo high-throughput profiling of CRISPR-Cpf1 activity. Nat. Methods 14, 153-159.

1. A method for training a predictive model for prime editing efficiency, comprising:
obtaining a dataset on a prime editing efficiency of pegRNAs according to cell types and prime editor types; and
training the predictive model using the dataset by deep learning, to establish relationships between the cell types the Prime Editor types and prime editing efficiency.

2. The method of claim 1, wherein the cell types comprise two or more selected from a group consisting of HEK293T, HCT116, DLD1, MDA-MB-231, A549, HeLa, and NIH3T3.

3. The method of claim 1, wherein the Prime Editor types comprise two or more selected from a group consisting of PE2, PE2max, PE2max-e, PE4max, PE4max-e, NRCH-PE2, NRCH-PE2max, and NRCH-PE4max.

4. The method of claim 1, wherein the prime editing efficiency of the pegRNAs refers to a ratio of edits induced by the pegRNA within the target sequence without unintended mutations.

5. The method of claim 1, wherein the dataset on a prime editing efficiency of a pegRNA is obtained by performing a method comprising:
preparing a plasmid library comprising oligonucleotides including a nucleotide sequence encoding the pegRNA, and a target nucleotide sequence of which targeted by the pegRNA;
introducing the plasmid library and the prime editor into cells;
performing deep sequencing on DNA obtained from the cells; and
analyzing prime editing efficiency from data obtained through deep sequencing.

6. The method of claim 1, wherein the dataset on a prime editing efficiency of pegRNAs comprises,
information on pairs of pegRNA-encoding sequences and target sequences for all types of edits with a length of 1-nt to 3-nt.

7. The method of claim 6, wherein a length of a Reverse Transcription Template (RTT) of the pegRNA is up to 50-nt, and
a length of a Primer Binding Site (PBS) of the pegRNA is in between 1-nt and 17-nt.

8. A method for predicting a prime editing efficiency, comprising:
obtaining information on a cell type, a Prime Editor type, and a target sequence; and
predicting the prime editing efficiency of a pegRNA by applying the information to the predictive model for prime editing efficiency, which is trained according to the method of claim 1.

9. The method of claim 8, wherein the information on the target sequence comprises a pair of an unedited sequence and an edited sequence.

10. The method of claim 8, wherein the information further comprises information regarding an editing length and an editing type.

11. The method of claim 8, wherein the method further comprises:
outputting a pegRNA sequence and a prime editing prediction score for the pegRNA.

12. An apparatus for predicting prime editing efficiency, comprising:
an input unit configured to receive information on a cell type, a prime editor type, and a target sequence; and
a prediction unit configured to apply the information to the predictive model for prime editing efficiency, which is trained according to the method of claim 1 to predict the prime editing efficiency of the pegRNA.

13. The apparatus of claim 12, wherein the information on the target sequence comprises a pair of an unedited sequence and an edited sequence.

14. The apparatus of claim 12, wherein the information further comprises information regarding an editing length and an editing type.

15. The apparatus of claim 12, wherein the apparatus further comprises an output unit configured to output a pegRNA sequence and a prime editing prediction score for the pegRNA.

16. A computer-readable recording medium on which a program is recorded, the program being configured to cause a computer to execute the method according to claim 8.

17. A method for training a predictive model for off-target prime editing efficiency, comprising:
obtaining a dataset on a prime editing efficiency of pegRNAs on an on-target sequence and off-target sequences; and
training the predictive model using the dataset by deep learning, to establish relationships between a feature affecting an off-target prime editing and the off-target prime editing efficiency.

18. The method of claim 17, wherein the off-target prime editing efficiency is a prime editing efficiency induced by the pegRNA on the off-target sequence.

19. The method of claim 17, wherein the dataset on a prime editing efficiency is obtained by performing a method comprising:
preparing a plasmid library comprising oligonucleotides comprising a nucleotide sequence encoding the pegRNA, and either an on-target or an off-target nucleotide sequence; and
introducing the plasmid library and a prime editor into cells;
performing deep sequencing on DNA obtained from the cells; and
analyzing prime editing efficiency from data obtained through deep sequencing.

20. The method of claim 17, wherein the feature affecting an off-target prime editing comprises one or more selected from a group consisting of a location of the mismatch, a number of the mismatch, a type of the mismatch, a length of a Primer Binding Site (PBS) of the pegRNA, a length of a Reverse Transcription Template (RTT).

21. A method for predicting an off-target prime editing efficiency, comprising:
obtaining information on a target sequence and a pegRNA sequence; and
predicting the off-target prime editing efficiency of a pegRNA by applying the information to the prime editing efficiency prediction model trained according to the method of claim 17.

22. The method of claim 21, wherein the information on the target sequence comprises an off-target sequence.

23. The method of claim 21, wherein the method further comprises:
outputting a prime editing prediction score for the pegRNA.

24. An apparatus for off-target prime editing efficiency, comprising:
an input unit configured to receive information on a target sequence and a pegRNA sequence; and
a prediction unit configured to apply the information to the predictive model, which is trained according to the method of claim 17 to predict the off-target prime editing efficiency of the pegRNA.

25. The apparatus of claim 25, wherein the information on the target sequence comprises an off-target sequence.

26. The apparatus of claim 24, wherein the apparatus further comprises an output unit configured to output an off-target prime editing prediction score for the pegRNA.

27. A computer-readable storage medium on which a program is recorded, the program being configured to cause a computer to execute the method according to claim 17.
